(19) United States
(12) Patent Application Publication
(10) Pub. No.: US 2020/0306295 A1
(43) Pub. Date: Oct. 1, 2020

(54) METHOD AND DEVICE FOR ANNIHILATION OF Staphylococcus aureus

(71) Applicant: Purdue Research Foundation, West Lafayette, IN (US)

(72) Inventors: Ji-xin Cheng, Newton, MA (US); Mohamed Seleem, West Lafayette, IN (US); Pu-Ting Dong, Boston, MA (US); Jie Hui, West Lafayette, IN (US)

(73) Assignee: Purdue Research Foundation, West Lafayette, IN (US)

(21) Appl. No.: 16/580,113

(22) Filed: Sep. 24, 2019

Related U.S. Application Data
(63) Continuation-in-part of application No. 16/139,127, filed on Sep. 24, 2018.
(60) Provisional application No. 62/561,765, filed on Sep. 22, 2017.

(51) Int. Cl. A61K 33/40; A61L 2/00; A61L 2/26; A61P 31/04; A61K 31/7028

(52) U.S. Cl. CPC A61K 33/40; A61L 2/0052; A61K 31/7028; A61L 2/26; A61P 31/04; A61L 2/0082

(57) ABSTRACT

Confronted with the rapid evolution and dissemination of antibiotic resistance, there is an urgent need to develop alternative treatment strategies for drug-resistant S. aureus, especially for methicillin-resistant S. aureus (MRSA). We report a photonic approach to eradicate MRSA through blue-light photolysis of staphyloxanthin (STX), an anti-oxidative carotenoid acting as the constituent lipid of the functional membrane microdomains of S. aureus. Our transient absorption imaging study and mass spectrometry unveil the photolysis process of STX. After effective STX photolysis by pulsed laser, cell membranes are found severely disorganized and malfunctioned to defense antibiotics, as unveiled by membrane permeabilization, membrane fluidification, and detachment of membrane protein, PBP2a. Consequently, our photolysis approach sensitizes MRSA to reactive oxygen species attack and increases susceptibility and inhibits development of resistance to a broad spectrum of antibiotics including penicillins, quinolones, tetracyclines, aminoglyco sides, lipopeptides, and oxazolidinones. The synergistic therapy, without phototoxicity to the host, is effective in combating MRSA both in vitro and in vivo in a mice skin infection model. Collectively, this staphyloxanthin-targeted phototherapy concept paves a novel platform to use conventional antibiotics as well as reactive oxygen species to combat multidrug-resistant S. aureus infections.

### CROSS REFERENCE

This application is a continue in part application for the U.S. application Ser. No. 16/139,127, filed on Sep. 24, 2018, which claims the benefit of U.S. Provisional Application No. 62/561,765, filed on Sep. 22, 2017. The contents of which is incorporated herein entirely.

### FIELD OF INVENTION

This disclosure relates to a novel method to deplete staphyloxanthin (STX) virulence factor in Staphylococcus aureus by STX photolysis via short-pulsed blue laser or low-level blue lights. This disclosure further relates to a novel synergistic treatment regimen between STX photolysis and antibiotic drugs or oxidative agents to treat S. aureus infections.

### BACKGROUND

Staphylococcus aureus is a major source of bacterial infections and causes severe health problem in both hospital and community settings. Of note, S. aureus becomes life-threatening especially when serious infections such as sepsis or necrotizing pneumonia occur. Though numerous antibiotics were once effective at treating these infections, S. aureus has acquired resistance which diminished the effectiveness of several classes of antibiotics. A classic example was the emergence of clinical isolates of MRSA strains in the 1960s that exhibited resistance to β-lactam antibiotics. More recently, strains of MRSA have manifested reduced susceptibility to new antibiotics and therapeutics such as vancomycin and daptomycin. Faced with the severe situation that introduction of new antibiotics into clinic could not keep pace with the rapid development of resistance, both the drug industry and health organizations are calling for alternative ways to combat the MRSA resistance.

Grounded on the increasing understanding of virulence factors in disease progression and host defense, anti-virulence strategies have arisen in the past decade as an alternative. In S. aureus, staphyloxanthin (STX), the yellow carotenoid pigment that gives its name, is a key virulence factor. This pigment is expressed for S. aureus pathogenesis and used as an antioxidant to neutralize reactive oxygen species (ROS) produced by the host immune system. Recent studies on cell membrane organization further suggest that STX and its derivatives condense as the constituent lipids of functional membrane microdomains (FMM), endowing membrane integrity and providing a platform to facilitate protein-protein oligomerization and interaction, including PBP2a, to further promote cell virulence and antibiotic resistance¹³. Therefore, blocking STX biosynthesis pathways has become an innovative therapeutic approach. Thus far, cholesterol-lowering drugs, including compound BPH-652 and statins, have shown capability of inhibiting S. aureus virulence by targeting the enzymatic activity, e.g. dehydrosqualene synthase (CrtM), along the pathway for STX biosynthesis. However, these drugs suffer from off-target issues, as human and S. aureus share the same pathway for biosynthesis of presqualene diphosphate, an intermediate used to produce downstream cholesterol or STX. Additionally, anti-fungal drug, naftifine, was recently repurposed to block STX expression and sensitize S. aureus to immune clearance. Despite these advances, all of these are still drug-based approaches to inhibit STX virulence, which require additional treatment time, accompany with serious side effects, show weak activities, and have higher risk for resistance development by targeting a single upstream biosynthetic enzyme, which will eventually prevent their clinical utilization.

### SUMMARY OF THE INVENTION

This disclosure provides treatment regimen and device of sensitizing a patient having antibiotic-resistant Staphylococcus aureus lesions. The lesion can be a wound or ear infection. The treatment regimen and device to carrying such regimen would have at least capability to provide short-pulsed blue laser or low-level blue lights to the infected lesion site to targeted photo-bleach the yellow pigment of staphyloxanthin (STX), wherein the short-pulsed blue laser or low-level blue lights create membrane pores, make membrane fluid, and detach membrane proteins. The regimen and device also provide an effective amount of oxidative agent as well as effective amount of antibiotics.

In some preferred embodiment the aforementioned treatment regimen and device uses hydrogen peroxide as the oxidative agent

In some preferred embodiment the aforementioned treatment regimen and device are to sensitize antibiotic-resistant Staphylococcus aureus selected from the group consisting of methicillin-resistant Staphylococcus aureus (MRSA), vancomycin-resistance S. aureus (VRSA), sulfamethoxazole/trimethoprim-resistant MRSA (Sul/Tri-R MRSA), and erythromycin-resistant MRSA (Ery-R MRSA).

In some preferred embodiment the aforementioned treatment regimen and device uses antibiotics selected from the group consisting of penicillins, quinolones, tetracyclines, aminoglycosides, lipopeptides, and oxazolidinones.

In some preferred embodiment the aforementioned treatment regimen and device prevents the development of S. aureus resistance to ciprofloxacin and ofloxacin.

In some preferred embodiment the aforementioned treatment regimen and device delays the development of S. aureus resistance to linezolid, tetracycline and tobramycin.

The disclosure provides a portable device to sequentially or simultaneously provide pulsed blue laser or low-level blue light to the lesion of a patient with antibiotic-resistant S. aureus infection and to administer an effective amount of antibiotics or hydrogen peroxide.

These and other features, aspects and advantages of the present invention will become better understood with reference to the following figures, associated descriptions and claims.

### BRIEF DESCRIPTION OF THE DRAWINGS

FIG. 1A-FIG. 1G. Photobleaching signature of MRSA under transient absorption microscopy. (FIG. 1A). Time-lapse images of wild type MRSA. Scalar bar=5 μm. Image acquisition time: 0.1 s. (FIG. 1B). Normalized time-course decreasing curve from wild type MRSA. (FIG. 1C). Time-lapse images of naftifine-treated MRSA. (FIG. 1D). Normalized time-course curve from wild type and naftifine-treated MRSA. (FIG. 1E). Image of CrtM mutant (t=0 s). (FIG. 1F-FIG. 1G). Image of wild type MRSA cluster (t=0 s, FIG. 1F) and CrtM mutant cluster (t=0 s, FIG. 1G). (FIG. 1H). Time-course curve from wild type cluster (FIG. 1F) and CrtM mutant cluster (FIG. 1G). White arrow: the interface between air and sample. Curve fitted by equation (1).

FIG. 2A-FIG. 2J. Mass spectrometry unveils the photochemistry of STX under blue light exposure. (FIG. 2A). Absorption spectrum of S. aureus extract along with the spectrum profile (blue) of blue light LED. (FIG. 2B). Blue light exposure bleaches S. aureus carotenoids. (FIG. 2C). Absorption spectrums of S. aureus extract at different blue light exposure time. (FIG. 2D). OD₄₇₀ of S. aureus extract decreases towards blue light exposure. Curve fitted by equation (1). (FIG. 2E). The correlation between m/z=819.5, m/z=721.5 and m/z=241.5 under different collision energies. (FIG. 2F). HPLC chromatograph of STX under different blue light exposure time. (FIG. 2G). Quantitative analysis of STX attenuation towards blue light exposure. Curve fitted by equation (1). (FIG. 2H-FIG. 2I). TOF-MS/MS analysis of S. aureus extract under different blue light exposure time. (FIG. 2H). Annihilation of STX ([M+Na⁺]) under blue light exposure. (FIG. 2I). Corresponding generation of unknown product during the photobleaching process of STX. (FIG. 2J). Potential photobleaching process of STX under blue light irradiation.

FIG. 3A-FIG. 31. Blue light and H₂O₂ synergistically eliminate MRSA in vitro. (FIG. 3A). The effect of blue light dose upon the survival percent of wild type MRSA. Blue light: 460 nm, 60 mW/cm². N=3. (FIG. 3B). Growth curve of untreated group and blue light-treated group. Blue light: 460 nm, 120 J/cm². (FIG. 3C). CFUs result of H₂O₂-only group and blue light and H₂O₂-treated group at different H₂O₂ concentrations. Blue light: 60 mW, 108 J/cm². H₂O₂ incubation time: 20 min. (FIG. 3D). CFUs result of H₂O₂-only group and blue light and H₂O₂-treated group. H₂O₂: 13.2 mM (0.045%), 20-min culture time. (FIG. 3E). Survival percent of MRSA from blue light-only group and blue light and H₂O₂-treated group at different H₂O₂ concentrations. Blue light: 470 nm, 108 J/cm². (FIG. 3F). Blue light sensitizes MRSA to H₂O₂ killing compared to S. epidermidis. Blue light: 470 nm, 60 J/cm². H₂O₂:13.2 mM, 5-min culture time. (FIG. 3G). Schematic cartoon illustrates how blue light assists ROS inside the macrophage cells to kill intracellular MRSA (not drawn to scale). Yellow dots: MRSA 400. Gray dots: MRSA 400 after blue light exposure. (FIG. 3H). CFUs results (n=3-6) of MRSA 400-infected macrophage cells from control (untreated), vancomycin-treated and blue light-treated groups. (FIG. 31). Statistical analysis of the CFUs results from different groups.

FIG. 4A-FIG. 4F. Blue light and H₂O₂ synergistically heal the MRSA-infected mice wound. (FIG. 4A). Schematic cartoon demonstrates the animal experimental process (not drawn to scale). (FIG. 4B). Physiological wounds condition of four different groups before, after treatment and after sacrifice. Red arrow: pus formation. (FIG. 4C). CFUs plates of the untreated group and the blue light and H₂O₂-treated group. (FIG. 4D). Statistical analysis of the CFUs results from five different groups. N=4-5. (FIG. 4E-FIG. 4F). In comparison with untreated group, the fold change from 200 kinds of cytokines in fusidic acid-treated group (FIG. 4E), blue light and H₂O₂-treated group (FIG. 4F).

FIG. 5. Schematic illustration of pump probe microscopy.

FIG. 6A-FIG. 6B. Oxygen dependence upon the photobleaching rate of S. aureus. (FIG. 6A). Time-course curves of MRSA with or without Na₂S₂O₄. Na₂S₂O₄, an oxygen scavenger. (FIG. 6B). Time-course curves of NRS with or without Na₂S₂O₄.

FIG. 7A-FIG. 7B. Power dependence upon the photobleaching intensity of S. aureus cluster under pump probe microscopy. Time-lapse curves of MRSA cluster towards probe intensity (FIG. 7A), and pump intensity (FIG. 7B).

FIG. 8A-FIG. 8C. Power dependence upon the photobleaching rate of S. aureus cluster under pump probe microscopy. Normalized time-lapse curves of MRSA cluster towards probe intensity (FIG. 8A), and pump intensity (FIG. 8B). (FIG. 4C). The power dependence of the time-course decay of MRSA upon the time spent to reach 1/e*Intensity.

FIG. 9A-FIG. 9D. Characteristics of photobleaching of β-carotene by pump probe microscopy. Time-lapse curves of β-carotene towards probe intensity (FIG. 9A), and pump intensity (FIG. 9B). Normalized time-lapse curves of β-carotene towards probe intensity (FIG. 9C), and pump intensity (FIG. 9D).

FIG. 10. Blue light LED apparatus.

FIG. 11A-FIG. 11F. Naftifine-treated S. aureus and CrtM mutant extract are immune to blue light exposure. Absorption spectrum of naftifine-treated S. aureus extract (FIG. 11A) and CrtM mutant extract (FIG. 11B) at different blue light exposure time. (FIG. 11C). OD₄₇₀ from carotenoids of naftifine-treated S. aureus and CrtM mutant change towards blue light irradiance. High-performance liquid chromatography chromatographs of STX from naftifine-treated S. aureus (FIG. 11D) and CrtM mutant (FIG. 11E) at different blue light exposure time. (FIG. 11F). Quantitative analysis of STX from naftifine-treated S. aureus and CrtM mutant during photobleaching process. Blue light, 470 nm, 90 mW (1 cm×1 cm) on the sample.

FIG. 12. The whole mass spectrum of STX.

FIG. 13A-FIG. 13B. Human whole blood effectively scavenges MRSA (FIG. 13A) and NRS (FIG. 13B) after photobleaching by pump probe microscopy. Blue light: 440 nm, 10 mW, 1 h light exposure time. Then for the control group, after exposure we culture them in the medium for 9 h. For the experimental group, bacteria were cultured in the fresh whole blood for 9 h.

FIG. 14. Staphyloxanthin sensitizes S. aureus towards blue light-based killing.

FIG. 15A-FIG. 15B. Blue light and H₂O₂ synergistically scavenge S. aureus inside the biofilm. (FIG. 15A). Fluorescence imaging of live S. aureus (Green, top), dead S. aureus (Red, middle) and merged live/dead S. aureus (Bottom) inside the biofilms of control group (left lane), Blue light-treated group (left middle), daptomycin-treated group (right middle lane) and blue light+H₂O₂-treated group (right lane). A live/dead viability kit was used to stain the cells inside the biofilms. Live: SYTO®9. Dead: Propidium iodide. Scalar bar=10 μm. Blue light: 30-min exposure, 360 J/cm². H₂O₂:13.2 mM, 20-min culture time, then quenched by 0.5 mg/mL catalase solution. (FIG. 15B). Statistical analysis of survival percent of S. aureus inside the biofilms at different groups. Survival %=N_{green}/(N_{green}+N_{red})·N_{green} and R_{red} represents the number of live S. aureus and dead S. aureus, respectively. Data are means (black) with standard error of mean (red). N=7-8, which was chosen from 7-8 different regions of interest, for each region, the size is the same as the image shown in (A).

FIG. 16A-FIG. 16B. Survival percent of MRSA depends on blue light dose and H₂O₂ culture time (FIG. 16A). Blue light: 1-2 min (12-24 J/cm²). H₂O₂: 13.2 mM. (FIG. 16B). Fix the H₂O₂ culture time (20 min). Fix the blue light irradiance (24 J/cm²).

FIG. 17A-FIG. 17B. Cytokine data analysis for blue light treated group (FIG. 17A) along with H₂O₂ treated group (FIG. 17B).

FIG. 18A-FIG. 18B. Nanosecond pulsed laser would enable dramatically improved STX photolysis efficiency, speed, and depth. (FIG. 18A) Nanosecond pulsed laser Fluence shows 6 orders of magnitude larger than CW LED on surface. (FIG. 18B) Photolysis process follows a bi-molecule behavior due to triplet-triplet annihilation process. This process highly depends on the molecule concentration. T* lifetime of STX is 10 us. Using high-intensity nanosecond laser (less than T* lifetime) to transiently populate STX molecules to their T* state, due to their high concentration within MRSA FMM, we can dramatically increase the photolysis efficiency and speed. Meanwhile we can also solve the heating issue.

FIG. 19A-FIG. 19C. Quantification of STX photlysis efficiency. Resonance Raman spectroscopy was applied to quantify STX in MRSA and to find the optimal wavelength for photolysis of STX. (FIG. 19A) STX show strong Raman signal at ˜1008, ˜1161, and ˜1528 cm-1 as its signal intensity linearly depends on local STX concentration, thus Raman peak amplitude at 1161 cm-1 was used for quantification of STX photolysis efficiency. (FIG. 19B). STX effectively bleached within the entire blue light range. (FIG. 19C) Bleaching speed and wavelength plot to determine the wavelength of 460 nm as optimal.

FIG. 20A-FIG. 20C. STX photolysis efficiency, and speed by blue light compared with CW LED under the same power and same dosage. Experiment conducted on MRSA solution sandwiched between two cover glass slides with ˜80 μm thickness. Signal drops both by nanosecond pulsed laser (FIG. 20A) and CW LED (FIG. 20B). (FIG. 20C). signal drop over treatment time shows pulsed laser is dramatically faster than CW LED. It takes pulsed laser only 4 minutes to bleach more than 80% of STX, while CW LED needs more than one hour. Even after long treatment, there is still large portion of STX left unbleached by CW LED, but pulsed laser enables nearly complete photolysis.

FIG. 21. With same dosage and same power, pulsed laser enable ˜5 fold improvement on treatment depth within one cell cycle treatment time. Samples were treated with pulsed laser or CW LED over real tissue sample with different thickness, and measured their Raman signal amplitude. Within one cell cycle treatment time, CW LED barely penetrates 300 um for 50% STX photolysis. While pulsed laser could reach 1 mm with more than 50% STX bleached.

FIG. 22. Resonance Raman spectroscopy to show photochemistry of STX. New Raman peaks shows up at 1133 cm⁻¹. A significant blue shift is observed at all three characteristic Raman peaks for STX. Up to 3, 6, 12 cm-1 for peaks at ˜1008, ˜1161, and ˜1528 cm⁻¹, respectively. These are also the evidences for photochemistry of STX.

FIG. 23A-FIG. 23C. Dramatically improved membrane permeability induced by STX photolysis in SYTOX green study. (FIG. 23A). For stationary-phase MRSA, its control groups has no obvious change in fluorescence intensity; while once MRSA cells are treated with laser, dyes can diffuse into the cells, binds to nucleic acids and then express significantly larger fluorescence intensity and significantly faster diffusion with longer treatment time. This means that laser treatment damages cell membrane integrity and induced significantly improved permeability to small molecules. Longer treatment time gives us, more bleached STX and more damaged cell membrane. (FIG. 23B). In log-phase cells with less STX on cell membrane, we see smaller fluorescence intensity and slower intracellular diffusion. (FIG. 23C). When cells are put back to nutritious medium they could not be able to recover even after two hours. This means cell membrane is damaged by STX photolysis. The more STX in cell membrane, more cell membrane is severely damaged after light treatment. Also cell membrane cannot be easily recovered after laser treatment.

FIG. 24. SYTOX Green confocal laser scanning microscopy. These images confirm that SYTOX green are indeed diffused into the cells. The three columns are fluorescence images, transmission channels, merged images. Below is 5 min treated MRSA cells. We can see significantly larger fluorescence intensity from cells in treated groups and the relative percentage of cells that has high fluorescence intensity is also significantly increased.

FIG. 25. FITC dextran structure used extensively in microcirculation and cell permeability. The molecular weight/size of FITC dextran is controllable in a wide range, with approximate stokes' radii as following:

| | |
|---|---|
| MW 4,000 | Aprprox. 14 Angstroms |
| MW 10,000 | Aprprox. 23 Angstroms |
| MW 20,000 | Aprprox. 33 Angstroms |
| MW 40,000 | Aprprox. 45 Angstroms |
| MW 70,000 | Aprprox. 60 Angstroms |
| MW 150,000 | Aprprox. 85 Angstroms |

FIG. 26. Membrane poration created via photolysis of staphylaxoxanthin. 2 min laser treatment could induce intercellular diffusion of FD70 with molecular weight of 70K and Stokes radius of 6 nm. 5 min treatment further induces dramatically increased intracellular diffusion of FD70. This indicates that membrane pore of 10 nm level has been created via STX photolysis.

FIG. 27A-FIG. 27B. Membrane poration created via photolysis of staphyloxanthin: FITCdextran structured illumination microscopy (SIM) imaging. Compared with untreated group, 5 min treated group showed significantly higher fluorescence intensity indicating dramatically increased intracellular diffusion of FD70. We then further increased the FD molecular weight to 500 k with stokes radius of ˜15 nm. But we didn't see significant increased intracellular diffusion. With these results, we can conclude that membrane pores via STX photolysis can be up to ˜10 nm-level size, but smaller than 30 nm. These pores are large enough for nearly every antibiotics target intracellular activity (even for nanoparticle). (FIG. 27A) SIM imaging (FIG. 27B) FD70 versus (FIG. 27C) FD500 treatment induced florescence intensity plot. FIG. 28. The intracellular diffusion of small-molecule antibiotics gentamicin. Gentamicin was conjugated with a dye, Texas red, so that intracellular gentamicin uptake can be tracked by confocal laser scanning microscopy. This first column is fluorescence channel; the second one is transmission and the third one is merged image. Compared with control group, 5 min light treated group shows significantly increased amount of gentamicin inside the cells. This result indicates that significantly increased cellular uptake of gentamicin can be achieved through large membrane pores created via laser treatment.

FIG. 29A-29B. PBP2a accumulation within membrane microdomains and its change induced by laser treatment. Immunostaining of sample for PBP2a with rabbit anti-PBP2a as the primary antibody and Cy5 as the secondary antibody. Structured illumination microscopy herein provides a lateral resolution about ˜130 nm and axial resolution of ˜150 nm. PBP2as are not uniformly distributed on cell membrane; rather they are highly concentrated within the membrane microdomains. The images are from one representative cell with roughly 7 FMMs. But after 5 min treatment, a significant drop is seen in its signal intensity (verified by confocal laser scanning microscope in the next slide). Compared with control group (FIG. 29A), PBP2a proteins (FIG. 29B) have the trend to disperse to surrounding membrane areas, which significantly reduce the signal contrast between FMM and its surrounding areas.

FIG. 30A-FIG. 30B. PBP2a is unanchored from membrane microdomains via photolysis of staphyloxanthin. (FIG. 30A) Consistent with structured illumination microscopic images, in confocal images, significant signal drop is observed from cells after laser treatment. Signal from 300 cells with and without laser treatment indicates that laser treatment could remove or unanchor a significant portion of PBP2a from cell membrane. (FIG. 30B). Preliminary western blotting results further confirmed this result as we have detected increased amount of PBP2a in supernatant.

FIG. 31A-FIG. 31B. MRSA with compromised membrane after laser treatment is able to recover in a time dependent manner. Pulsed laser was used to treat stationary-phase MRSA with different time. Immediate CFU counting showed MRSA are just dramatized by laser, not immediately killed. (FIG. 31A) When MRSA are cultured in PBS without nutrition, the longer treatment time, the more MRSA cells are killed, indicating that dramatized MRSA dies without nutritious medium. But 2-log reduction by laser treatment alone is not complete. Remarkably, when MRSA cells were cultured after laser treatment in nutritious medium, most of the MRSA cells are able to recover. Quantification of recover time is determined by culturing them in nutritious medium with different time and then followed by CFU counting. (FIG. 31B). Comparing recovery curves of untreated, 5 min treated, 10 min treated groups shows recovery time depends on laser treatment time. MRSA cells need 0.5-1 h to recover after 5 min laser treatment and 1-2 h after 10 min laser treatment.

FIG. 32. Laser treatment has synergy with conventional antibiotics. Laser treatment induces large membrane pores and removes significant amount of PBP2a from cell membrane. It created a great opportunity to find synergy with conventional antibiotics, thus to revive conventional antibiotics. Here are some preliminary data we have got on antibiotics. Compared with the control, 5 min laser treatment alone kills MRSA by less than 1 log. The concentration of antibiotics applied here is 10 MIC for all antibiotics. No obvious killing for all tested antibiotics except for daptomycin, the last resort antibiotics for MRSA. But when combined laser treatment with antibiotics, very obvious synergy for nearly every class of antibiotics is observed. These include cefotaxime, gentamicin, ciprofloxacin, oxacillin. Gentamicin has show more than 2 log reduction.

FIG. 33. Photo-toxicity and photo-selectivity No phototoxicity has been detected with 460 nm nanosecond pulsed laser with illumination time up to 10 mins. With volunteer's arm illuminated by the same laser beam, no heating is reported and no any observable photo damage. The power and dosage applied are well below ANSI safety limit for skin exposure (ANSI MPE: 0.02 J/cm2; 0.2 W/cm2 for 300 min).

FIG. 34A-FIG. 34I. Photophysics and photochemistry of pulsed laser photolysis of STX. (FIG. 34A) (Left) Schematic of MRSA colony (or MRSA solution or STX extract solution) treated by nanosecond pulsed laser in a wide-field illumination configuration. (Right) Digital images of MRSA colony over laser treatment time to show golden color fading phenomenon. Image were recorded with sample placed on a transparent glass cover slide over a black paper. (Bottom) STX molecular structure. 0 refers diameter of bacterial colony. (FIG. 34B) Resonance Raman spectroscopy of MRSA colony over 460 nm nanosecond pulsed laser treatment time (measured on the same colony). Numbers indicate major Raman peak positions. (FIG. 34C) Resonance Raman spectroscopy of MRSA and S. aureus ΔCrtM colonies. The images show the color of spun-down cells. (FIG. 34D) Spectroscopic study of STX photolysis efficiency with nanosecond pulsed laser power of 50 mW and treatment time of 5 min. STX photolysis efficiency is quantified by Raman peak amplitude at 1161 cm⁻¹. (FIG. 34E) Raman quantification of STX abundance in multidrug-resistant S. aureus cells before and after 5 min laser treatment (460 nm). Bacterial strains include vancomycin-resistance S. aureus (VRSA), sulfamethoxazole/trimethoprim-resistant MRSA (Sul/Tri-R MRSA), and erythromycin-resistant MRSA (Ery-R MRSA). (FIG. 34F) STX photolysis kinetics of MRSA colony by nanosecond pulsed laser and CW LED under the same illumination power, area, and center wavelength (460 nm). Solid black curve is the fitting result by a second-order photobleaching model. (FIG. 34G) Resonance Raman spectroscopy of STX in MRSA colony with or without long time-treatment by nanosecond pulsed laser and CW LED at 460 nm highlighting STX photolysis induced Raman peak shifts and the generation of new Raman peak. Numbers indicate Raman peak positions before and after light treatment. (FIG. 34H) STX photolysis kinetics of STX solution by nanosecond pulsed laser and CW LED under the same illumination power, area, and center wavelength (460 nm). STX solution were extracted directly from MRSA cells. (FIG. 34I) STX photolysis kinetics of MRSA colony placed beneath a tissue layer with various thickness by nanosecond pulsed laser and CW LED under the same illumination power, area, and center wavelength (460 nm). The inset shows the schematic of experimental scheme. Ah indicates the thickness of tissue layer. CW, continuous wave. The cells used were all cultured to reach 3-day stationary phase. N=3 for all the above measurements.

FIG. 35A-FIG. 34F. Photophysics and photochemistry of pulsed laser photolysis of STX. (FIG. 34A) Absorption spectroscopy of MRSA solution over 460 nm nanosecond pulsed laser treatment time. All measurements were performed on the same sample. (FIG. 34B) Digital images of bacterial colonies of multidrug-resistant S. aureus isolates before and after 5 min laser treatment (460 nm). Bacterial strains include vancomycin-resistance S. aureus (VRSA), sulfamethoxazole/trimethoprim-resistant MRSA (Sul/Tri-R MRSA), and erythromycin-resistant MRSA (Ery-R MRSA). Image were recorded with sample sandwiched between two transparent glass cover slides over a black paper. (FIG. 34C) Resonance Raman spectroscopy of MRSA colony over 460 nm CW LED treatment time (measured on the same colony). Numbers indicate major Raman peak positions. (FIG. 34D) Resonance Raman spectroscopy of STX solution over 460 nm nanosecond pulsed laser treatment time (measured on the same colony). Numbers indicate major Raman peak positions. These peak positions at 1031 and 1524 cm⁻¹ and the peak amplitude change at 1031 cm⁻¹ are different from that of MRSA colony, indicating different chemical environment for STX in extract solution and MRSA membrane (FIG. 34E) Resonance Raman spectroscopy of STX solution by nanosecond pulsed laser and CW LED under the same illumination power, area, and center wavelength (460 nm) highlighting a similar STX photolysis efficiency. STX solution were extracted directly from MRSA cells. (FIG. 34F) STX photolysis kinetics of MRSA colony by nanosecond pulsed laser under the same dosage but different illumination power (460 nm). CW, continuous wave. N=3 for all the above measurements.

FIG. 36A-FIG. 36K. First mechanism for photo-disassembly of membrane microdomains: membrane permeabilization. (FIG. 36A) Schematic of membrane permeability mechanism via pulsed laser photolysis of STX. (FIG. 36B) Real-time intracellular uptake kinetics of SYTOX green by stationary-phase MRSA with or without pulsed laser treatment. (FIG. 36C) Confocal fluorescence images of intracellular uptake of SYTOX green by stationary-phase MRSA cells with or without pulsed laser treatment. (Top) fluorescence images. (Bottom) corresponding transmission images. (FIG. 36D) Statistical analysis of fluorescence signal from MRSA cells in (FIG. 36C) from each treated group with N≥300 per group. (FIG. 36E) Real-time intracellular uptake kinetics of SYTOX green by stationary-phase S. aureus ΔCrtM with or without pulsed laser treatment. (FIG. 36F) Confocal fluorescence images of intracellular uptake of gentamicin-Texas red by stationary-phase MRSA cells with or without pulsed laser treatment. (Top) fluorescence images. (Bottom) Corresponding transmission images. (FIG. 36G) Statistical analysis of fluorescence signal of MRSA cells in (FIG. 36F) from each treated group with N≥300 per group. (FIG. 36H) Fluorescence detection of ciprofloxacin uptake by stationary-phase MRSA with or without pulsed laser treatment. (FIG. 36I) Structured illumination microscopic images of FD500 uptake by stationary-phase MRSA cells with or without pulsed laser treatment. Insets shows representative images of FD500 distribution on single cell after 5 min laser treatment. Fluorescence detection of (FIG. 36J) FD70 and (FIG. 36K) FD500 uptake by stationary-phase MRSA with or without pulsed laser treatment. MW, molecular weight. Scale bar, 5 μm for (FIG. 36C, FIG. 36F, FIGS. 36I) and 0.5 μm for zoom-in images in (FIG. 36I). N=3 for all the above measurements.

FIG. 37A-FIG. 37D. First mechanism for photo-disassembly of membrane microdomains: membrane permeabilization. (FIG. 37A) Confocal fluorescence images of intracellular uptake of SYTOX green by stationary-phase MRSA cells with or without pulsed laser treatment showing STX photolysis-mediated SYTOX green uptake. (Top) fluorescence images. (Bottom) corresponding transmission images. Scale bar, 5 μm. (FIG. 37B) Real-time intracellular uptake kinetics of SYTOX green by stationary-phase MRSA after 10 min pulsed laser treatment with or without followed by 2-hour culturing. (FIG. 37C) Real-time intracellular uptake kinetics of SYTOX green by log-phase MRSA with or without pulsed laser treatment. (FIG. 37D) Fluorescence detection of gentamicin-Texas red uptake by stationary-phase MRSA with or without pulsed laser treatment. N=3 for all the above measurements.

FIG. 38A-FIG. 38G. Second mechanism for photo-disassembly of membrane microdomains: membrane fluidification. (FIG. 38A) Schematic of membrane insertion of DiIC₁₈ induced by gel/rigid-to-liquid/fluid phase change. Molecular structure of DTIC is shown. (FIG. 38B) (Left and middle columns) fluorescence images of DiIC₁₈ foci formation for groups including log-phase MRSA and stationary-phase MRSA with or without laser treatment. (Right colum) zoom-in fluorescence images of MRSA cells with different foci number on each cell. Fluorescence from DiIC₁₈, red; transmission, grey. Scale bar, 5 μm for (left and middle columns) and 1 μm for (right column). Statistical analysis of foci number on cells from each group in (FIG. 38B): (FIG. 38C) log-phase and stationary-phase MRSA without laser treatment; (FIG. 38D) stationary-phase MRSA with different laser treatment time. N≥800/group. (FIG. 38E) (Top row) fluorescence images of daptomycin-BODIPY on stationary-phase MRSA with or without laser treatment. (Middle row) representative zoom-in images of the upper row. (Bottom row) corresponding transmission channels. Scale bar, 5 μm for (top and bottom row) and 0.5 μm for (middle row). (FIG. 38F) Statistical analysis of fluorescence signal intensity from MRSA cells in (FIG. 38E) with or without laser treatment with N≥800. (FIG. 38G) Schematic of antibiotic membrane insertion mechanism via pulsed laser photolysis of STX.

FIG. 39. Second mechanism for photo-disassembly of membrane microdomains: membrane fluidification. Molecular structure of daptomycin-BODIPY.

FIG. 40A-FIG. 40M. Third mechanism for photo-disassembly of membrane microdomains: membrane protein detachment. (FIG. 40a) Schematic of PBP2a protein structure and location relative to STX enriched membrane microdomain. (FIG. 40b, FIG. 40c) SIM images of PBP2a via immunostaining on MRSA cells in (FIG. 40b) 2-D and (FIG. 40c) 3-D. Intensity color bar applies to (FIG. 40b, FIG. 40c). (FIG. 40d, FIG. 40e) SIM images of PBP2a immunostaining on MRSA cells in (d) 2-D and (FIG. 40e) 3-D after 5 min laser treatment. Intensity color bar applies to (FIG. 40d, FIG. 40e). Scale bar, 2.0 μm for (FIG. 40b, FIG. 40d) and 0.5 μm for (FIG. 40c, FIG. 40e). (FIG. 40f) Statistical analysis of signal intensity from MRSA cells with or without laser treatment with N≥100. (FIG. 40g) Statistical analysis of PBP2 coefficient of variation on MRSA cells w/0 laser treatment with N≥100. (FIG. 40h) Western blot of PBP2a on MRSA pellets and its supernatant for groups with different laser treatment time. Numbers indicate the integrated signal intensity. Pageblue staining of the same samples was used as a loading control. (FIG. 40i) Schematic of PBP2a disassembly and detachment mechanism via pulsed laser photolysis of STX. (FIG. 40j) Self-assembled microphase separated domain structures of modeled membrane after 10 μs molecular dynamics simulation. Full-length STX lipids, red; cardiolipin lipids, blue; PBP2a peptides, yellow. (FIG. 40k) Final configuration of modeled membrane with truncated STX after 10 μs molecular dynamics simulation. Color scheme also applies to (FIG. 40j). Water and ions are made invisible for clarity for (FIG. 40j, FIG. 40k). Scale bar, 5 nm for (j, k). (FIG. 40l) RDFs of PBP2a peptides relative to the full-length STX and cardiolipins (FIG. 40L) and truncated STX and cardiolipins (FIG. 40M). Numbers on the plot indicate the locations of the first peak for each RDF.

FIG. 41A-FIG. 41G. Third mechanism for photo-disassembly of membrane microdomains: membrane protein detachment. (FIG. 41a) Statistical analysis of foci number on stationary-phase MRSA cells with N≥300. (FIG. 41b) Quantification of PBP2a dispersion by calculating coefficient of variation from each cell from standard deviation (σ) and mean (μ) of the plotted signal intensity. Scale bar, 0.5 μm. (FIG. 41c-FIG. 41f) Coarse-grained representations of the (FIG. 41c) full-length STX, (FIG. 41d) truncated STX, (FIG. 41e) cardiolipin (CDL), and (FIG. 410 PBP2a transmembrane peptide. (FIG. 41g) Initial configuration of modeled membrane. Full-length STX lipids, red; cardiolipin lipids, blue; PBP2a peptides, yellow. Water and ions are made invisible for clarity. Scale bar, 5 nm.

FIG. 42A-FIG. 42T. Photo-disassembly of membrane microdomains potentiates a broad spectrum of conventional antibiotics. Time-dependent killing of stationary-phase (FIG. 42a) MRSA and (FIG. 42b) S. aureus ΔCrtM cells in phosphate-buffered saline after different laser treatment time. Post-exposure effect of stationary-phase (FIG. 42c) MRSA and (FIG. 42d) S. aureus ΔCrtM after different laser treatment time. (FIG. 42e-FIG. 42l) Checkerboard assay results for synergy evaluation between laser treatment and different classes of antibiotics: (FIGS. 42e, f) tetracycline, (FIGS. 42g, h) ofloxacin, (FIGS. 42i, j) linezolid, and (FIGS. 42k, l) oxacillin. (FIGS. 42f, h, j, l) Selected cell growth curves acquired from corresponding checkerboard assay results of each antibiotic. (FIG. 42m) Viability of stationary-phase MRSA after laser treatment alone or in combination with daptomycin with different concentrations followed by 6-hour incubation in phosphate-buffered saline. (FIG. 42n) Time-dependent killing of stationary-phase MRSA in phosphate-buffered saline after laser treatment alone or in combination with 10 MIC daptomycin. (FIG. 42o) Viability of stationary-phase MRSA after laser treatment alone or in combination with gentamicin with different concentrations followed by 6-hour incubation in phosphate-buffered saline. (FIG. 42p) Time-dependent killing of stationary-phase MRSA in phosphate-buffered saline after laser treatment alone or in combination with 10 MIC gentamicin. (FIG. 42q) Time-dependent killing of stationary-phase vancomycin-resistant S. aureus (VRSA) strain in phosphate-buffered saline for four different treatment groups. (FIG. 42r) Efficiency of laser treatment alone or in combination with daptomycin on MRSA-caused mice skin infection model. (FIG. 42s) Hematoxylin and eosin stained histology evaluation of phototoxicity on mice skin. The mice used and treatment procedure applied were the same as that of (FIG. 42r) but without MRSA infection on the skin. (FIG. 42t) Viability of human keratinocyte cells over different laser treatment time to evaluate phototoxicity. N=5 for CFU enumeration for in vivo mice study. Dap, daptomycin; Tob, tobramycin. N=3 for the rest CFU enumeration, for checkerboard assay of each antibiotic and for phototoxicity evaluation on both human cells and in vivo mice.

FIG. 43A-FIG. 43L. Photo-disassembly of membrane microdomains potentiates a broad spectrum of conventional antibiotics. Post-exposure effect of (FIG. 43a) stationary-phase and (FIG. 43b) log-phase MRSA cells after different laser treatment time highlighting that the growth delay induced by laser treatment is dependent on STX abundance in MRSA cells. (FIG. 43c) Post-antibiotic effect of stationary-phase MRSA cells for ofloxacin, oxacillin, and gentamicin relative to the control. (FIGS. 43d-g) Checkerboard assay results for synergy evaluation between laser treatment and different classes of antibiotics: (FIGS. 43d, e) ciprofloxacin, (FIGS. 43f, g) vancomycin. (FIGS. 43e, g) Selected cell growth curves acquired from corresponding checkerboard assay results for each antibiotic. Time-dependent killing of stationary-phase (FIG. 43h) sulfamethoxazole/trimethoprim-resistant MRSA (Sul/Tri-R MRSA) and (FIG. 43i) erythromycin-resistant MRSA (Ery-R MRSA) in phosphate-buffered saline for four different treatment groups. (FIG. 43j) Time-dependent killing of stationary-phase MRSA in fresh human whole blood with or without 10 min laser treatment. (FIG. 43k) Time-dependent killing of stationary-phase MRSA in phosphate-buffered saline supplemented with different concentration of hydrogen peroxide after different laser treatment time. (FIG. 43l) Schematic of experiment design for mice skin infection model. N=3 for checkerboard assay of each antibiotic. N=3 for CFU enumeration.

FIG. 44A-FIG. 44M. Photo-disassembly of membrane microdomains inhibits resistance development to conventional antibiotics. (FIG. 44a) Representative resonance Raman spectroscopy of STX in stationary-phase MRSA cells at different time checkpoints for the group treated with 10 min laser alone over 48-day serial passage. (FIG. 44b) STX abundance in stationary-phase MRSA cells over 48-day serial passage for groups with or without 10 min laser alone quantified via Raman peak amplitude at 1161 cm⁻¹. (FIG. 44c) Images of spun-down cells in (FIG. 44b) after 48-day serially passage showing STX pigmentation. (FIG. 44d) MIC fold change of SPL1 for different classes of antibiotics after 48-day serial passage. (FIGS. 44e, h, j, k, l, m) Resistance acquisition over 48-day serial passage in the presence of sub-MIC levels of antibiotics with or without 10 min laser treatment: (FIG. 44e) ciprofloxacin, (FIG. 44h) ofloxacin, (FIG. 44j) linezolid, (FIG. 44k) tetracycline (FIG. 44l) tobramycin, (FIG. 44m) ramoplanin. (FIGS. 44f, i) Images of spun-down cells from (FIG. 44e) and (FIG. 44h), respectively, after 48-day serially passage showing STX pigmentation. (FIG. 44g) Checkerboard assay of SPA0_48 showing that 16 min laser treatment completely eliminated cell growth. N=3 for checkerboard assay study, Raman spectra, and STX quantification. SPO, serial passage without any treatment; SPL1 and SPL2, serial passage in independent duplicate with laser treatment alone; SPA0, serial passage with sub-MIC antibiotic treatment alone; SPLA1 and SPLA2, serial passage in independent duplicate with 10 min laser plus sub-MIC antibiotic treatment. The numbers after these abbreviations denote serially passage days.

FIG. 45A-FIG. 45C. Photo-disassembly of membrane microdomains inhibits resistance development to conventional antibiotics. (FIG. 45a) STX abundance in stationary-phase MRSA cells over 48-day serial passage in the presence of sub-MIC levels of ciprofloxacin with or without 10 min laser treatment. quantified via Raman peak amplitude at 1161 cm⁻¹. Resistance acquisition over 48-day serial passage in the presence of sub-MIC levels of antibiotics with or without 10 min laser treatment: (FIG. 45b) oxacillin, (FIG. 45c) gentamicin.

TABLE 1. Statistical Results of Fold Change of 200 of Cytokines from Four Different Groups. SEM Means Standard Error of Mean.

Table 2. Minimum Inhibitory Concentrations of Selected Antibiotics Against the Tested Bacterial Strains. N=3 for Each Measurement.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

Superbug infection has become a great threat on global heath, especially the pace of resistance acquisition is faster than the clinical introduction of new antibiotics. Consider this reason, WHO listed top 12 superbugs that poses the greatest threat to human health. MRSA is one of them. Our research is focusing on this superbug and using photo-disseambly of membrane microdomains to revive a broad spectrum of antibiotics against MRSA.

There are a variety of disease that are caused by S. aureus or MRSA infection. These can be skin and soft tissue infection, wound infection, diabetic ulceration and sepsis. No matter what kind of disease, once infected by S. aureus or MRSA, routine antibiotics treatment is applied to these infections.

However, S. auresu has various strategies to develop antibiotics resistance. Hence there is a battle between S. aureus evolution and antibiotics development. There are some major defense strategies of S. auresu. First, S. aureus can develop and secrete new enzymes to deactivate antibiotics. For example, beta-lactamase can break the structure of beta-lactamase susceptible beta-lactam antibiotics. Second, they can also change the target of antibiotics. For example, S. aureus can generate PBP2a proteins for cell wall synthesis when other PBPs are deactivated by beta-lactam antibiotics. Third, they can pump out antibiotics to reduce intracellular concentration that target intracellular activities, e.g. fluoroquinolones that inhibit DNA synthesis and tetracycline that inhibit RNA activity. Fourth, they can trap antibiotics and make them less active. Or they can acquire resistance through other genetic mutations. Besides resistance development, S. aureus can also develop other strategies. They can hide inside host cells, forming biofilms or become persisters that are metabolically inactive thus can tolerant high concentration antibiotics. In recently years, persisters have drawn more and more attentions, as they are particularly responsible for chronic/recurrent infections that are hard treat.

Due to these resistance development strategies, the discovery of novel antibiotics is currently not keeping pace with the emergence of new superbug. Nearly every existing antibiotic has found their resistant strain and the last new antibiotic was clinically introduced 33 years ago. There are multiple reasons for this. Firstly, antibiotics mis-use or overuse on human and livestock. Secondly, it normally takes roughly 10 years and needs a lot of money to develop a new antibiotic. Third, resistant strains will be soon found for new antibiotics after a few years. So pharmaceutical companies cannot justify to develop new antibiotics. But still health organizations are calling for novel antibiotics or alternative approaches to combat superbug infections.

There are a few emerging antibiotics or new strategies to treat S. aurous infections. Nature 556, 103-107 (2018) by Eleftherios Mylonakis Group demonstrates that synthetic retinoid antibiotics can be developed as new antibiotics to kill MRSA by disrupting their membrane lipid bilayer. These antibiotics also work synergistically with gentamicin due to the disrupted membrane. As another example, Nature 473, 216-220 (2011) by James Collins group demonstrated that some specific metabolic stimuli (e.g. mannitol or glucose) can generate proton motive force to enable trans-membrane uptake of aminoglycoside antibiotics to kill MRSA persisters. These two strategies highlight the importance of intracellular delivery of antibiotics. This can be done either by disrupting cell membrane or using metabolic stimuli.

Grounded on the increasing understanding of virulence factors in disease progression and host defense, anti-virulence strategies have arisen in the past decade as an alternative. In S. aureus, staphyloxanthin (STX), the yellow carotenoid pigment that gives its name, is a key virulence factor. This pigment is expressed for S. aureus pathogenesis and used as an antioxidant to neutralize reactive oxygen species (ROS) produced by the host immune system′². Recent studies on cell membrane organization further suggest that STX and its derivatives condense as the constituent lipids of functional membrane microdomains (FMM), endowing membrane integrity and providing a platform to facilitate protein-protein oligomerization and interaction, including PBP2a, to further promote cell virulence and antibiotic resistance. Therefore, blocking STX biosynthesis pathways has become an innovative therapeutic approach. Thus far, cholesterol-lowering drugs, including compound BPH-652 and statins, have shown capability of inhibiting S. aureus virulence by targeting the enzymatic activity, e.g. dehydrosqualene synthase (CrtM), along the pathway for STX biosynthesis. However, these drugs suffer from off-target issues, as human and S. aureus share the same pathway for biosynthesis of presqualene diphosphate, an intermediate used to produce downstream cholesterol or STX. Additionally, anti-fungal drug, naftifine, was recently repurposed to block STX expression and sensitize S. aureus to immune clearance. Despite these advances, all of these are still drug-based approaches to inhibit STX virulence, which require additional treatment time, accompany with serious side effects, show weak activities, and have higher risk for resistance development by targeting a single upstream biosynthetic enzyme, which will eventually prevent their clinical utilization.

Another example is to repurpose existing drug. Cell 171, 1354 (2017) by Danile Lopez Group demonstrates that cholesterol lowering drug, statin, can be used to reduce staphyloxanthin derived lipids within membrane microdomains, thus interferes PBP2a oligomerization and inhibit MRSA penicillin resistance. The paper introduced concept of functional membrane microdomains (FMM). Staphyloxanthin (STX)-derived lipids are the constituent lipids for FMM. Flotillins are the scaffold protein within the FMM. Many protein cargoes (e.g. PBP2a) are anchored and oligomerized within FMM. Once treated with statin, STX-derived lipids will be dramatically reduced. Therefore, PBP2a complex will be disassembled and its expressing amount is reduced, so penicillin resistance can be inhibited. Without being limited by any theory, it is proposed that STX is the constituent lipid for FMM and it is highly concentrated within FMM. PBP2a complex is within STX-enriched FMM.

These examples all have the potential to be used in the clinic. However, all these approaches still rely on new drugs or stimuli. S. aureus still can potentially develop resistance to these approaches. Also drugs, e.g. statin, takes long time to make MRSA susceptible to beta-lactam antibiotics.

In this study, we unveil that staphyloxanthin is the molecular target of photons within the entire blue wavelength range, demonstrating an unconventional way to deplete STX photochemically. Grounded on the STX photolysis kinetics, a short-pulsed blue laser was further identified to strip off this pigment with high efficiency and speed in wide field. In contrast to drug-based approaches, this photonic approach depletes the final product, STX, swiftly in a drug-free manner. More significantly, this disruption, enabled by the pulsed laser, fundamentally disorganizes and further malfunctions FMM as unveiled by increased membrane fluidity, ample membrane permeability, and PBP2a protein detachment, simultaneously and immediately after exposure. These membrane damages inhibit PBP2a deactivation of penicillins and facilitate the intracellular delivery and membrane insertion of conventional antibiotics, specific to their mechanisms of action. As a result, photo-disassembly of FMM restores the susceptibility and inhibits resistance development to a broad classes of conventional antibiotics against MRSA. Additionally, this work further deciphers the structural and functional properties of STX-enriched membrane microdomains for antibiotic resistance, thus providing a strategy to tackle antibiotic resistance by targeting STX virulence.

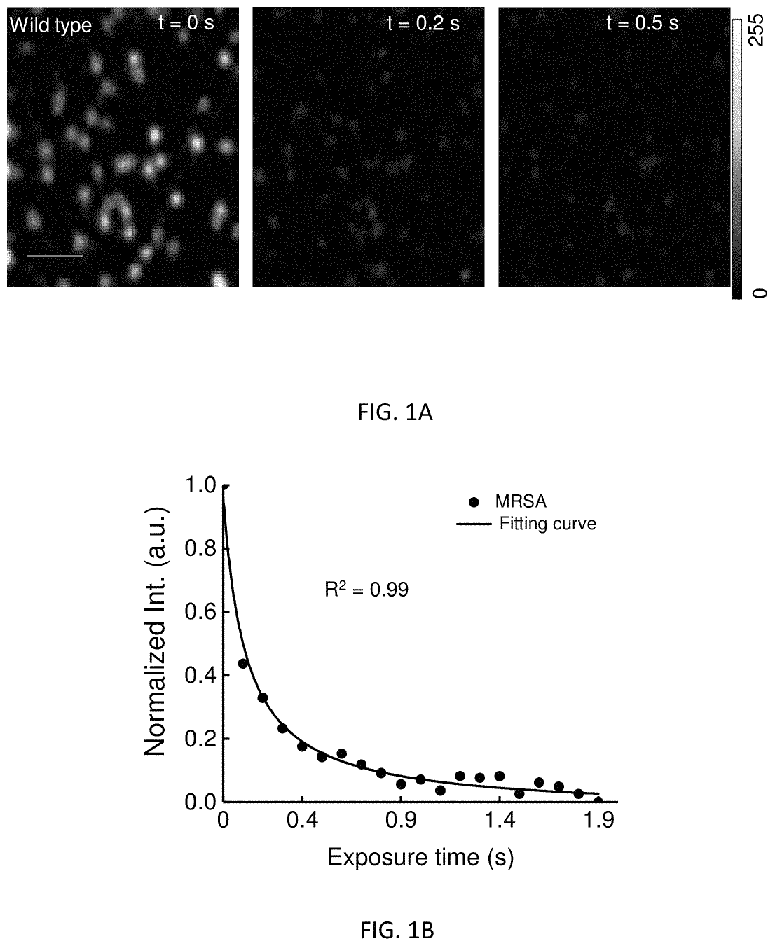

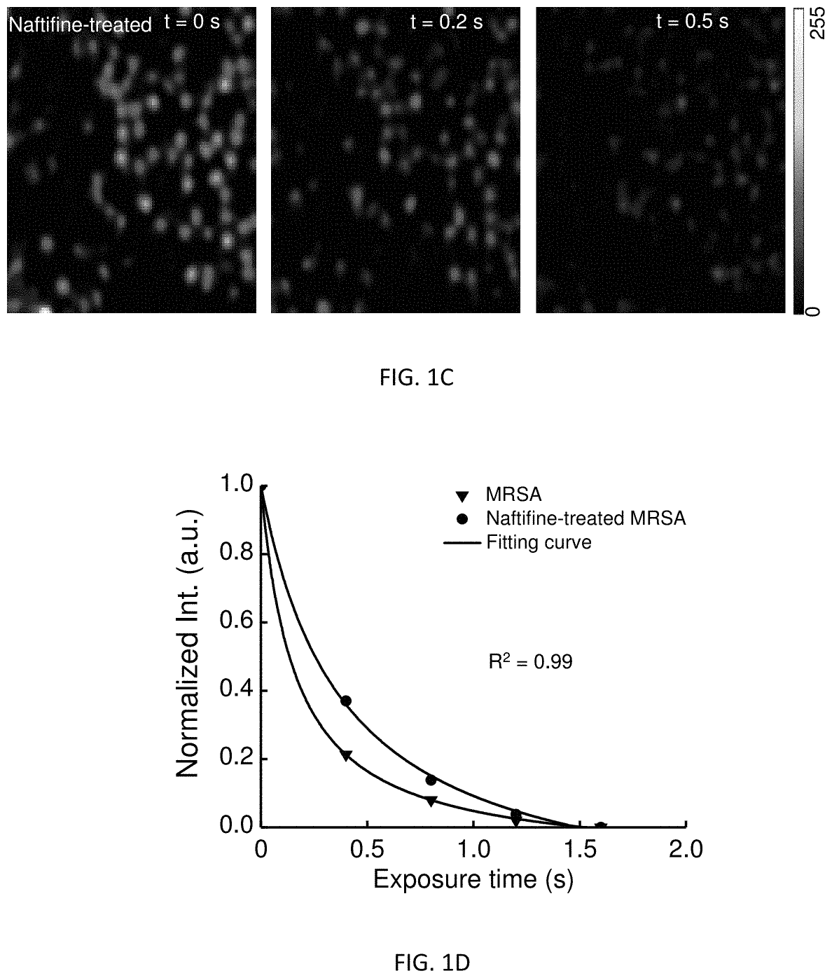

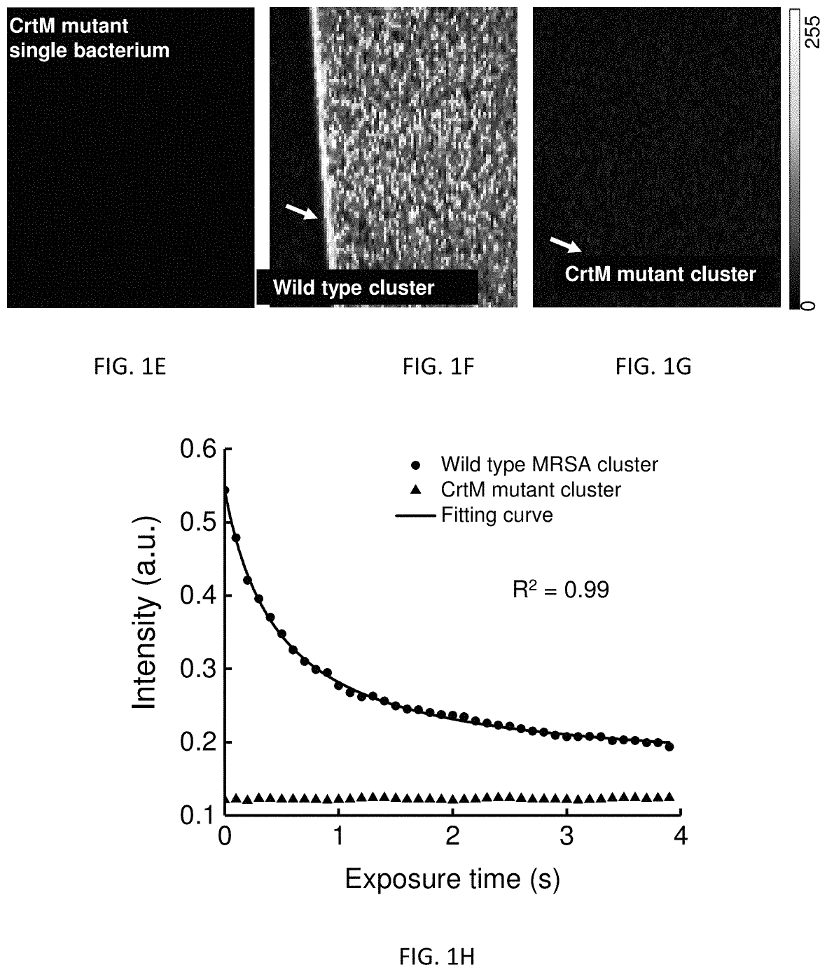

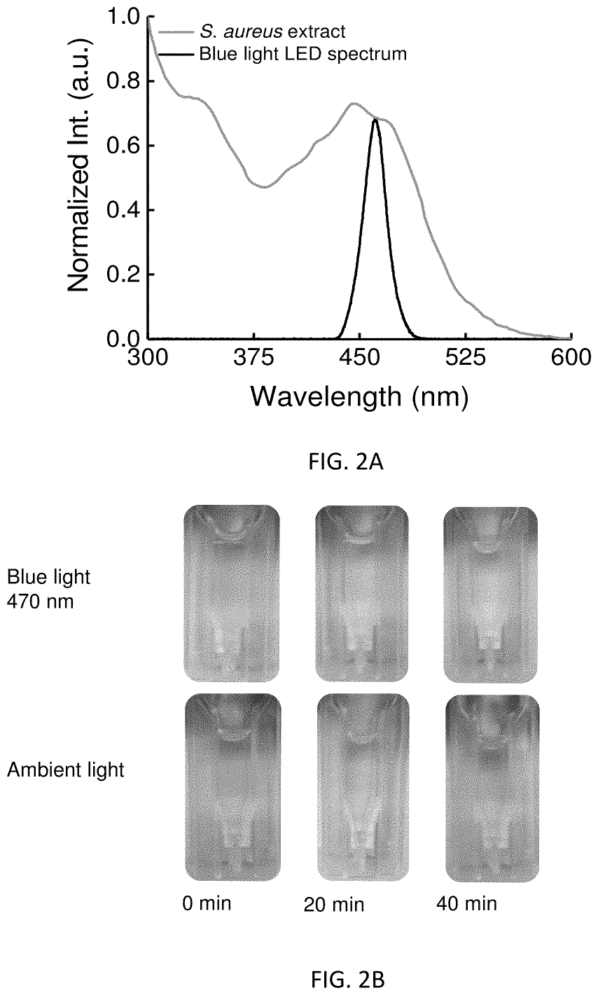

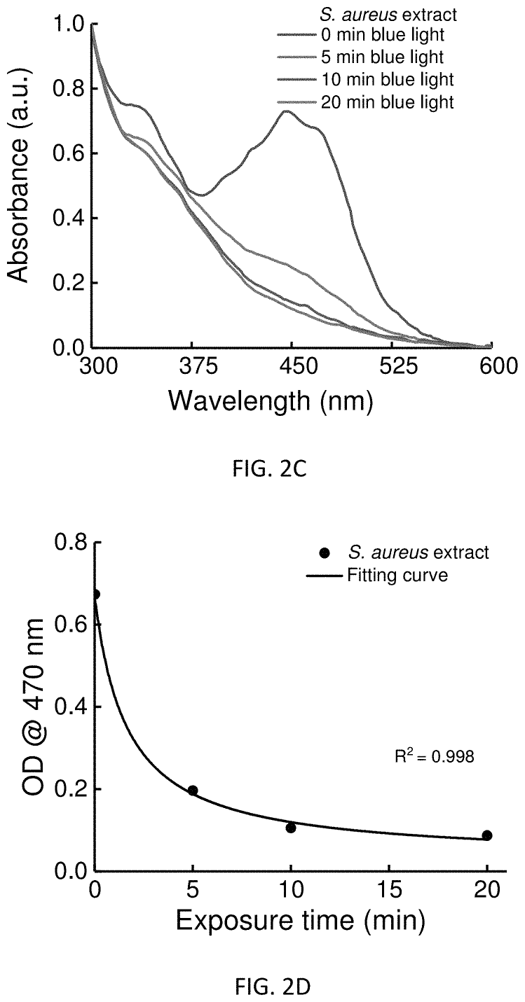

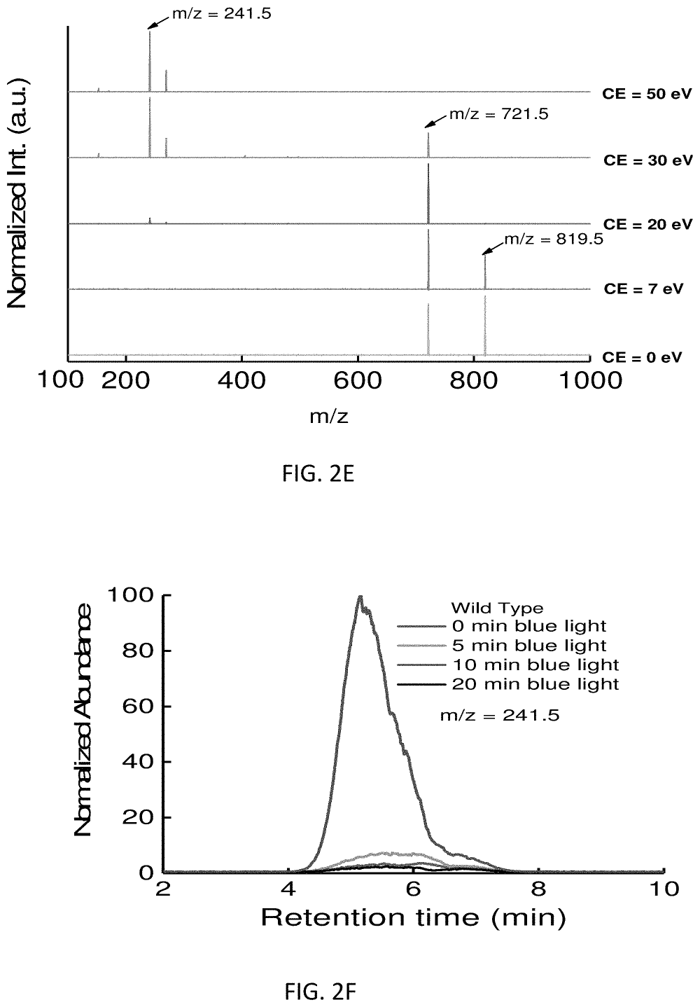

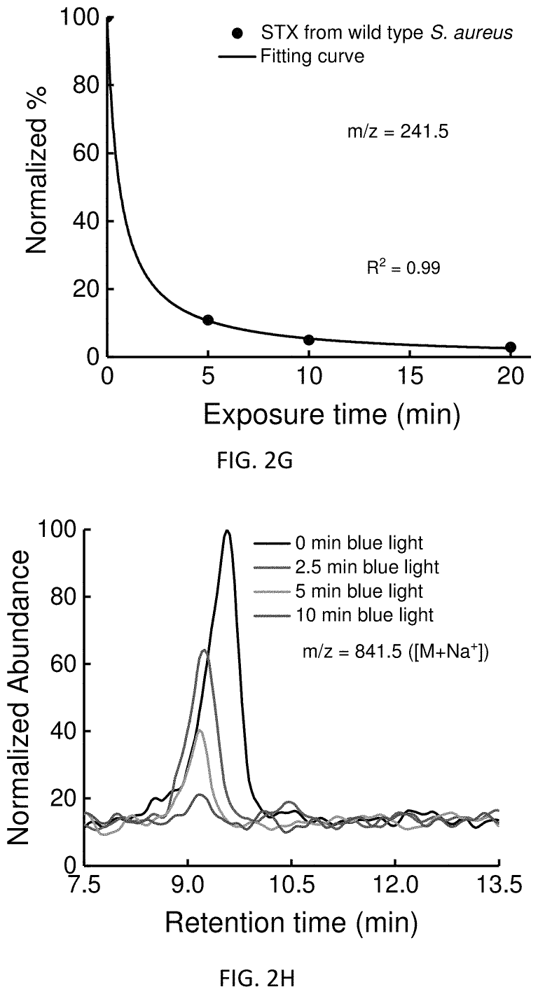

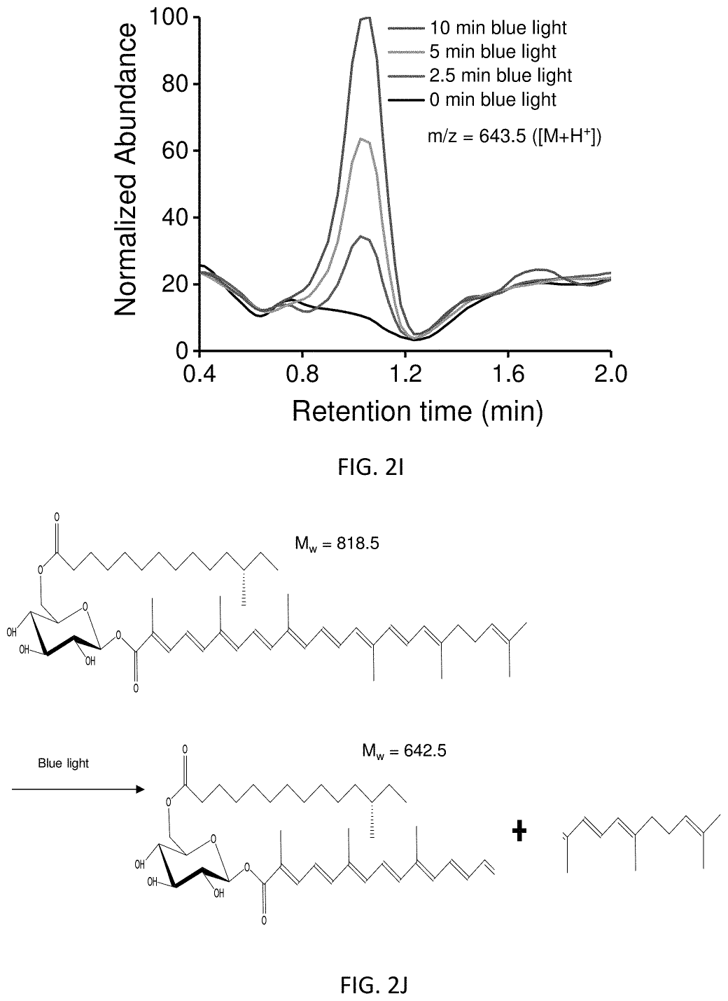

This disclosure started with an initial unexpected discovery that STX is prone to bleaching by blue light. Our group accidentally found the photobleaching phenomena on MRSA under transient absorption microscope. FIGS. 1A-1H show that transient absorption signal from MRSA dropped dramatically over time with zero delay between pump and probe pulses. To understand which chromophore is responsible for photobleaching, we treated MRSA cells with a FDA-approved drug to block the biosynthesis of STX. We observed a significantly smaller signal intensity and slower photobleaching decay compared to control group. For S. aureus mutant, there is no detectable signal. So, here we found that the gold pigment, staphyloxanthin is identified to be responsible for the observed photobleaching. FIGS. 2A-2J show that the absorption spectrum of STX and a continuous-wave (CW) LED for wide-field photolysis of STX at 460 nm. In FIGS. 2A-2J the golden color disappears upon photolysis. But it takes long time, one-hour level, to treat MRSA by using a CW LED. CW LED also suffers from superficial treatment depth and significant heating issue, making this technology very changeling for clinical translation.

In order to bypass these hurdles, we propose using photons, a non-drug approach, to resensitize MRSA to conventional antibiotics. This approach only takes several minutes to resensitize these antibiotics and also it can save a broad spectrum of antibiotics. Particularly, we use pulsed laser to induce nano-scale pores and unanchor PBP2a proteins within membrane microdomains.

A drug-free photonic approach to eliminating MRSA through effective photobleaching of STX, an indispensable anti-oxidative pigment residing inside the bacterium cell membrane is disclosed herein. Initially we attempted to differentiate MRSA from non-resistant S. aureus (NRSA) by transient absorption imaging (see methods) of intrinsic chromophores. Intriguingly, once the cultured S. aureus was placed under microscope, the strong signal which was measured at zero delay between the 520-nm pump and 780-nm probe pulses, irreversibly attenuated over second time scale. This process was captured in real time (FIG. 1A).

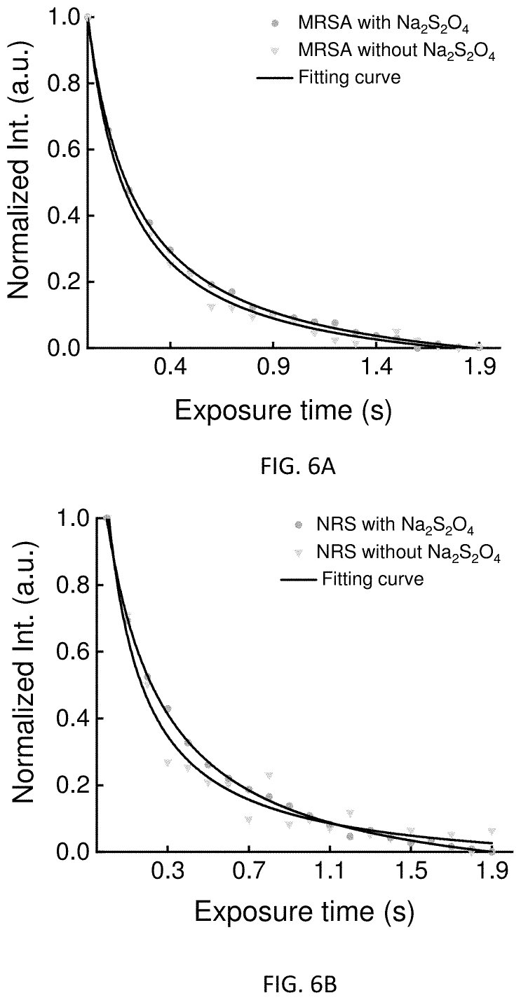

Without being limited by any theory, we made hypothesis that a specific chromophore in S. aureus is prone to photobleaching under our transient absorption imaging setting. To verify the photobleaching phenomenon, we fitted the time-course curve (FIG. 1B) with a previously described photobleaching model:

$$y = {y}_{0} + A * \frac{exp ( - \frac{t}{{τ}_{1}} )}{1 + \frac{{τ}_{1}}{{τ}_{2}} * ( 1 - exp ( - \frac{t}{{τ}_{1}} ) )} , \qquad ( 1 )$$

where t is the duration of light irradiation, y is the signal intensity, y₀ and A are constants, τ₁ and τ₂ are the bleaching constants for the first and second order bleaching, respectively. Derivation is detailed in supplementary text. First order bleaching happens at low concentration of chromophores (usually involved in singlet oxygen, τ₂=∞). Second order bleaching takes place when quenching within surrounding chromophores dominates (τ₁=∞). Strikingly, this photobleaching model fitted well the raw time-course curve (R²=0.99) with τ₂=0.16 s (τ₁=∞). Moreover, we found that oxygen depletion (Na₂S₂O₄: oxygen scavenger) has negligible effect on the bleaching speed since oxygen-depleted MRSA had a τ₂ of 1.36±0.12 s and τ₂ in control group was 1.00±0.20 s (FIG. 6A). The same phenomenon was observed in NRSA (FIG. 6B). Collectively, these data support a second order photobleaching process.

Next, we asked what chromophore inside S. aureus account for the observed photobleaching. It is known that carotenoids are photosensitive due to the conjugated C═C double bonds (14, 15). Therefore, we hypothesized that STX, a carotenoid pigment residing in the membrane of S. aureus, underwent photobleaching in our transient absorption study. To test this hypothesis, we treated MRSA with naftifine, a FDA-approved antifungal drug for STX depletion (11), the treated MRSA exhibited lower signal intensity (FIG. 1C) and slower photobleaching speed (FIG. 1D). FIG. 1D shows that τ₂ of naftifine-treated MRSA (τ₂=0.39±0.07 s, τ₁=∞) is 2.5 times longer than that of wild-type MRSA (τ₂=0.16±0.01 s, τ₁=∞). To further confirm the involvement of STX, we studied the CrtM mutant which is STX deficient (16) and observed no transient absorption signal (FIG. 1E). To avoid the systematic error aroused by single bacterium measurement, we investigated the clustered bacteria. It turned out that CrtM mutant cluster (FIGS. 1, G and H) only exhibited background induced by cross-phase modulation (17), whereas the wild-type MRSA cluster showed a sharp contrast against the background (FIG. 1F) and a fast photobleaching decay (FIG. 1H). Taken together, these data show that STX in S. aureus accounts for the observed photobleaching.

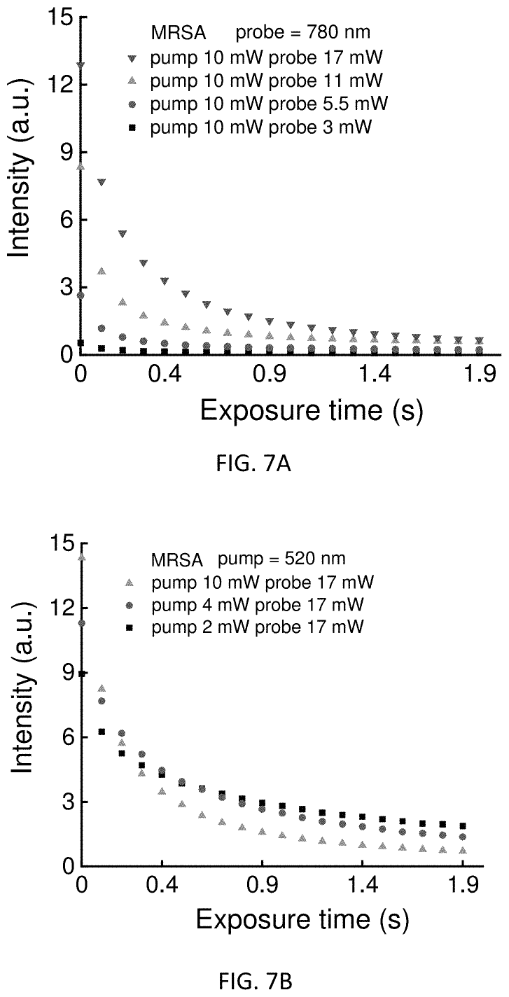

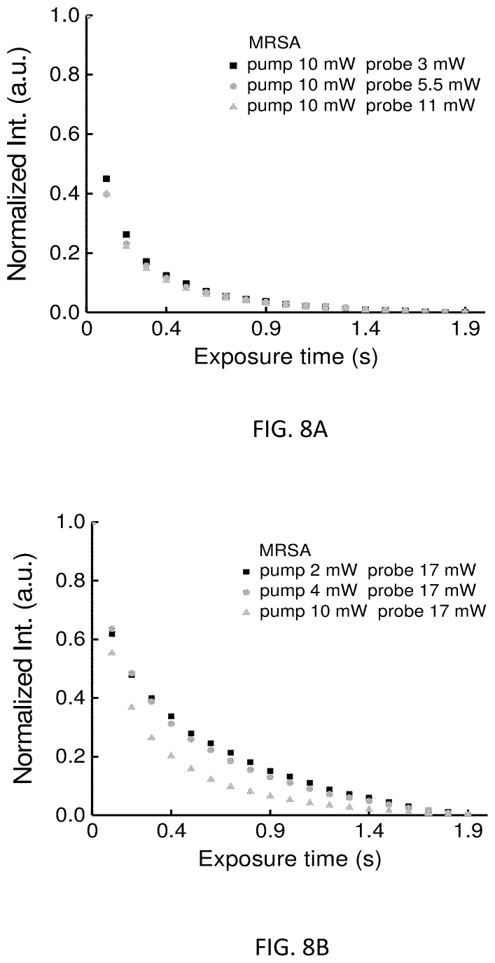

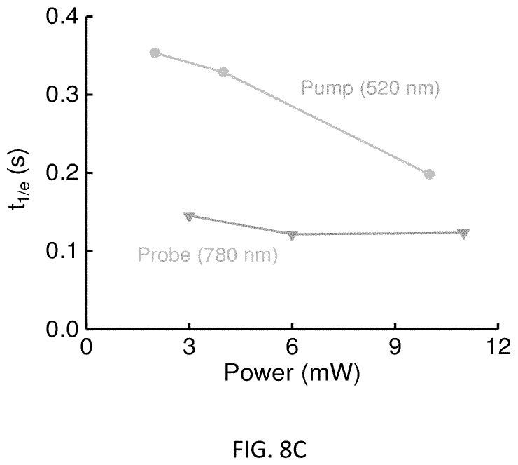

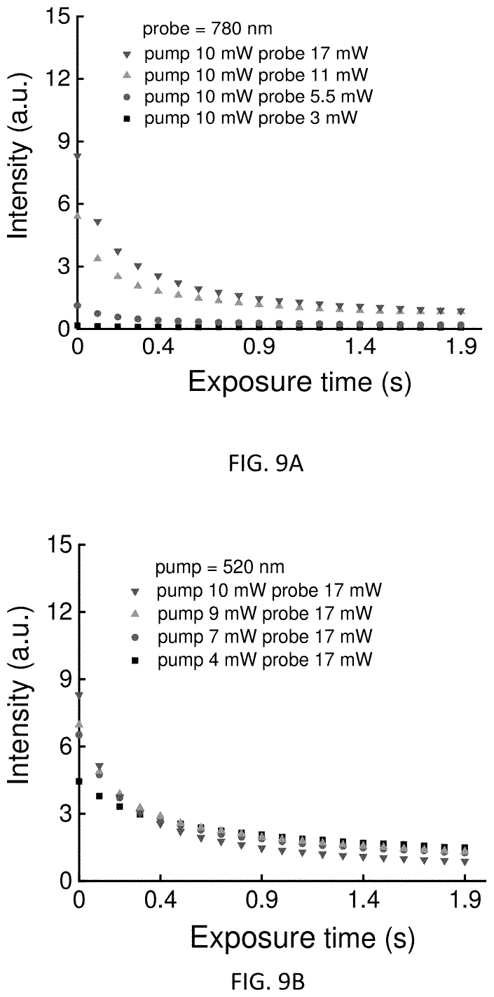

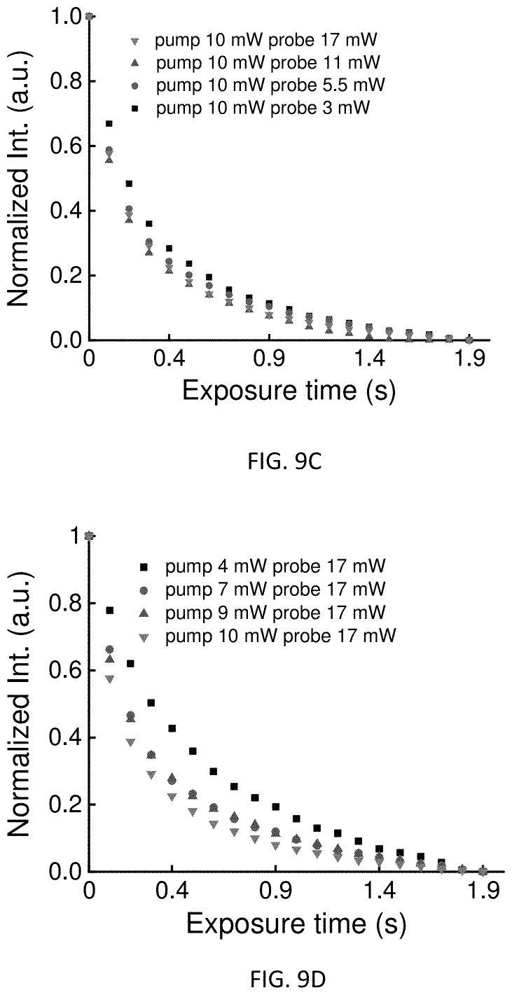

In our transient absorption study, when changing the 520-nm pump irradiance while fixing the probe intensity, both the photobleaching speed and transient absorption intensity altered drastically (FIGS. 7B and 8B), whereas the alteration of 780-nm probe irradiance only effected the transient absorption intensity but not the photobleaching speed (FIGS. 7A and 8A). Of note, β-carotene, which has a similar structure to STX, exhibits similar behavior such as laser irradiance dependence (FIGS. 9A and 9B) and wavelength selection (FIGS. 9C and 9D). These findings collectively imply a strong dependence of photobleaching efficacy on wavelength selection (FIG. 8C), which is consistent with the fact that photobleaching is linked to the absorption of chromophore (18).

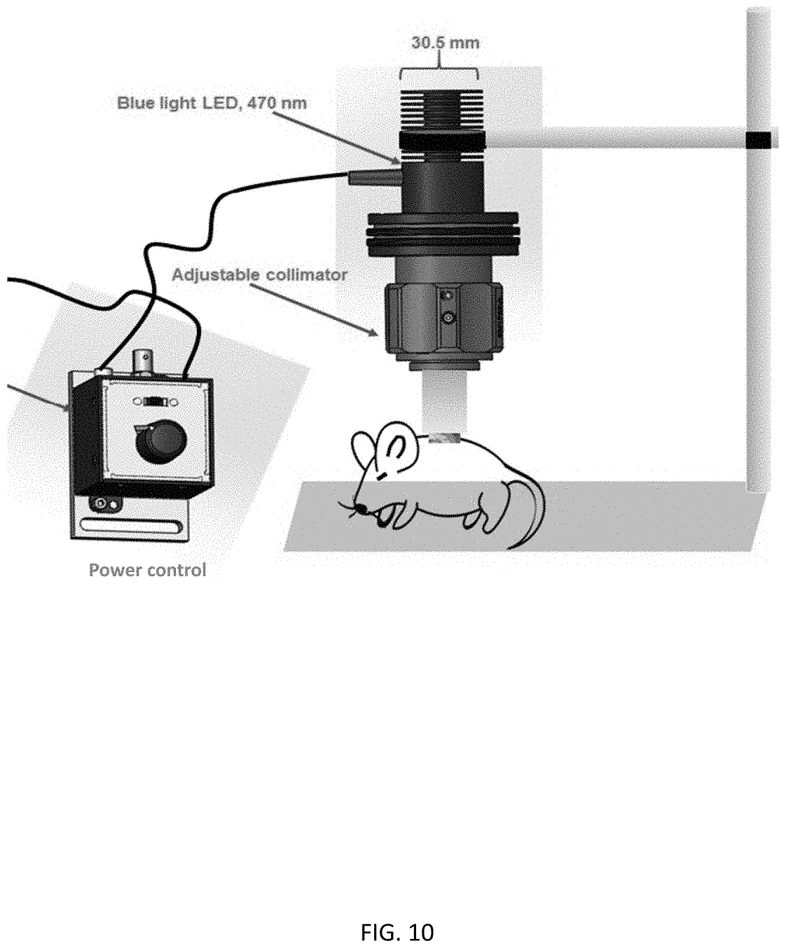

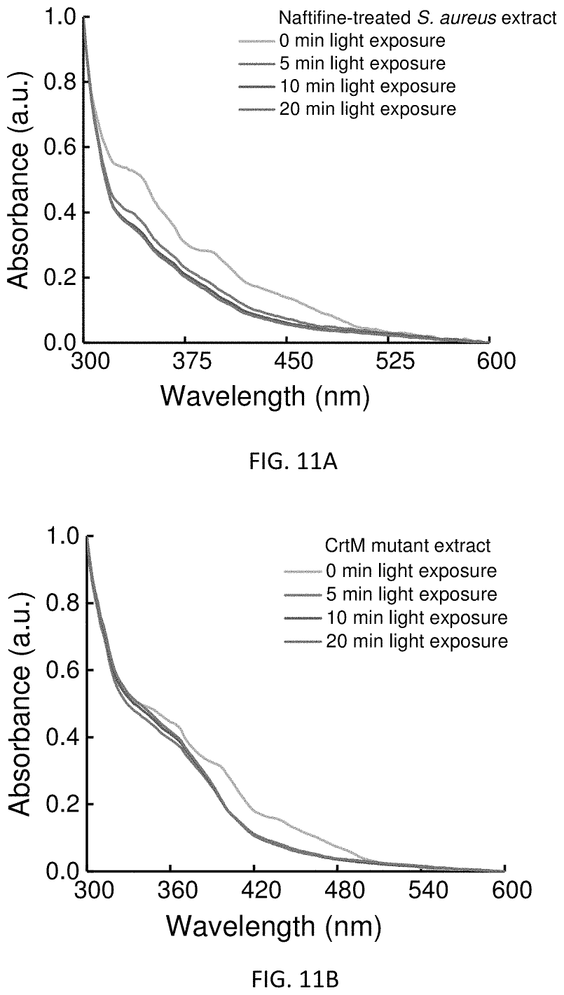

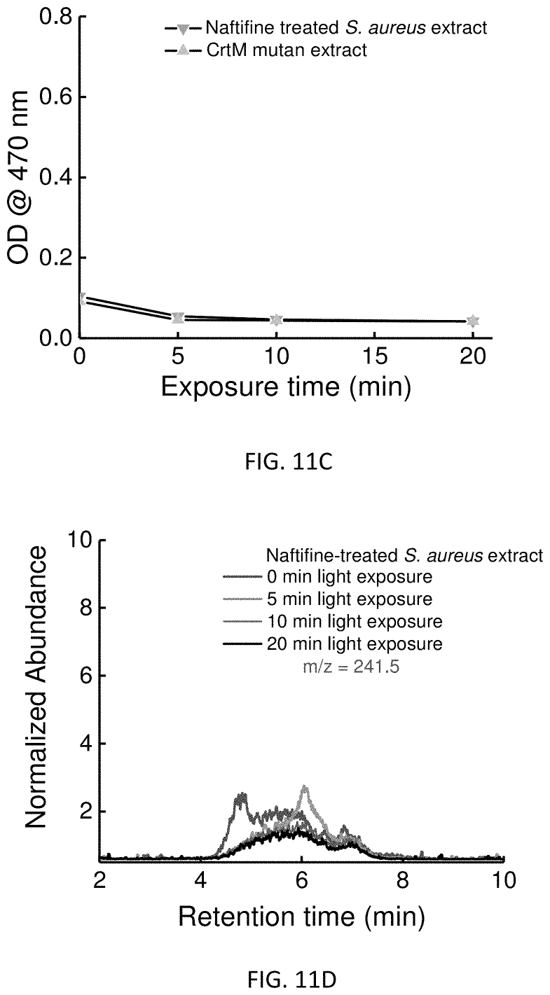

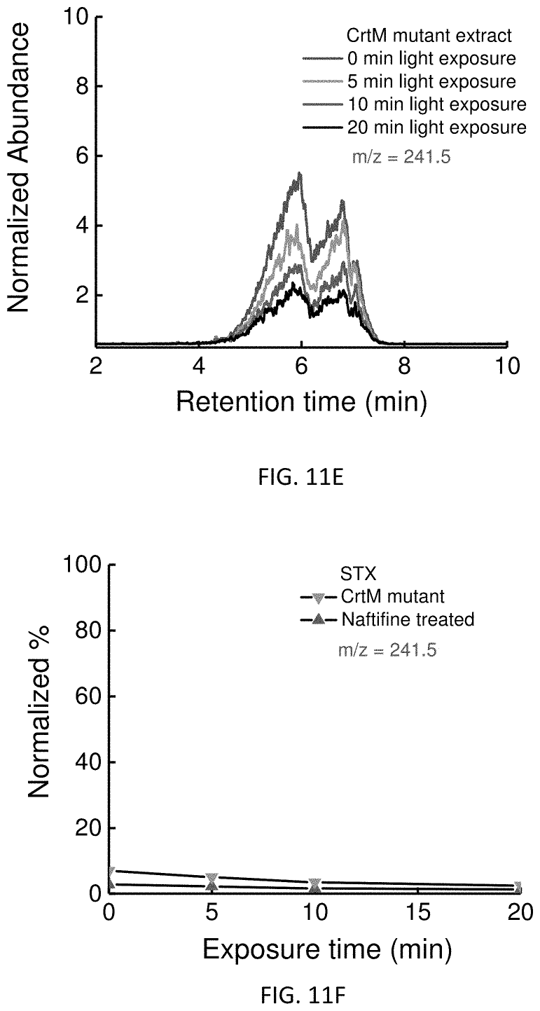

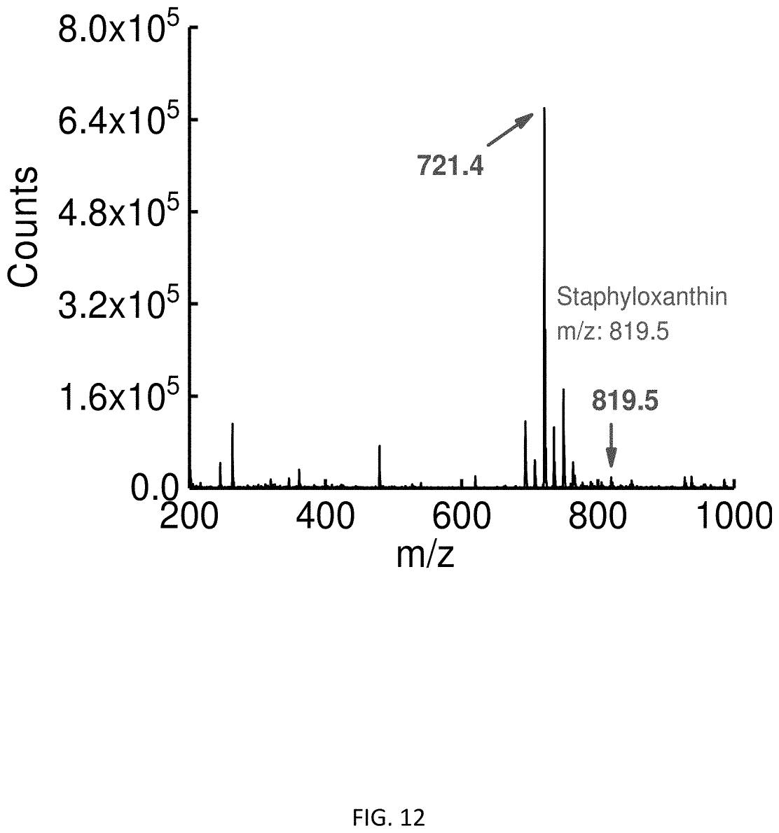

To identify the optimal wavelength for bleaching STX, we measured the absorption spectrum of MRSA extract (FIG. 2A), which shows peaks around 450 nm. This result triggered us to build a portable blue light LED for wide-field bleaching of STX (FIG. 10). We exposed MRSA extract to blue light irradiance (90 mW) for different time lengths. It turned out that the distinctive golden color from S. aureus carotenoids disappeared within 30-min exposure (FIGS. 2, B and C), whereas group under ambient light remained unchanged (FIG. 2B). In addition, the decreasing absorption trace of S. aureus can be well fitted with equation (1) (FIG. 2D). We also found that extracts from naftifine-treated MRSA or CrtM mutant were immune to blue light exposure, indicated by no changes in the absorption spectra (FIGS. 11 A to C). These findings conclude that STX is prone to photobleaching under blue light irradiance.

To quantitate the photobleaching process, we exploited mass spectrometry to target STX during blue light irradiation. FIG. S8 exhibits the MS spectrum of S. aureus extract with m/z ranging from 200 to 1000 at a certain collision energy. An abundant peak appeared at m/z=721.4. Moreover, m/z=819.5 ([M+H±]) is consistent with the molecular weight of STX (M, =818.5 g/mol). To find out the relationship between m/z=721.4 and m/z=819.5, we gradually increased the collision energy from 0 to 20 eV. As shown in FIG. 2(E), we found that m/z=721.4 is a product ion from m/z=819.5. These data proved that STX is the major species among the S. aureus extract. When the collision energy was higher than 20 eV, m/z=241.5, which comes from the precursor ion m/z=721.4, became dominant and presented a stable marker (FIG. 2E). Thus, to accurately quantify the amount of STX versus blue light dose, we targeted the HPLC area specifically from ion m/z=241.5 (retention time: 5.5 min, FIG. 2F). FIG. 2G depicts the blue light bleaching dynamics of STX. With 5-min (27 J/cm²) exposure, only 10% of STX (from 3.29×10⁹ bacteria) is left (FIG. 2G). A dose of 54 J/cm² attenuated all STX from ˜10⁹ bacteria. As a control, naftifine-treated and CrtM mutant S. aureus extract had negligible response to blue light exposure (FIGS. 11 D to F).

Next, we employed TOF-MS/MS (see methods) to elucidate how STX is decomposed during the photobleaching process. Different from the m/z=819.5 in HPLC-MS/MS, STX showed a peak at m/z=841.5 (FIG. 2H), which is an adjunct between STX and Na⁺ (Retention time: 9.5 min). Degradation of STX would definitely bolster the aggregation of some chemical segments. Through screening, we found a patch of the products existing after STX photobleaching (data not shown here). Notably, a significantly intensity-increased peak at m/z=643.4 (FIG. 2I), which is the adjunct between part of the STX along with H⁺ FIG. 2J illustrates the breakdown of conjugated C═C bonds of STX during blue light-activated photobleaching process.

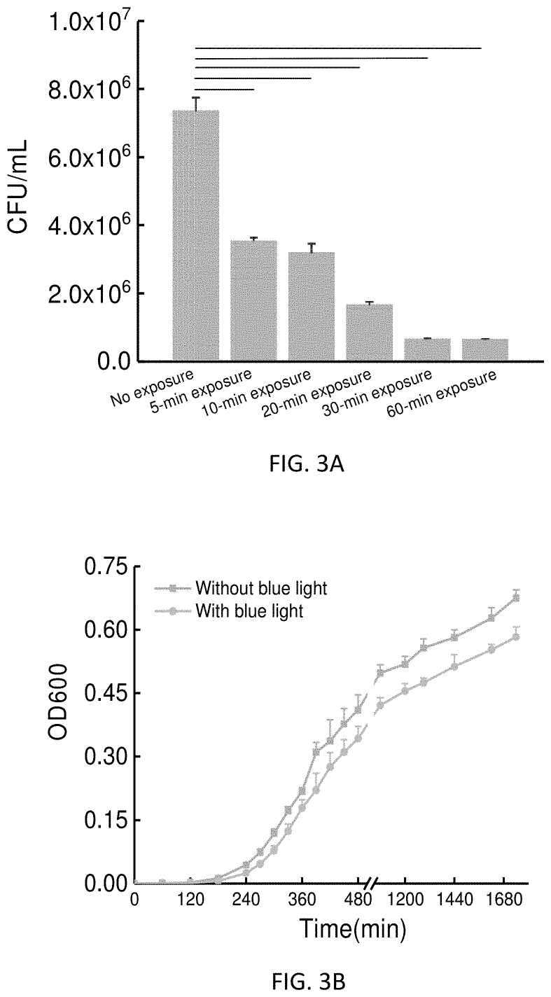

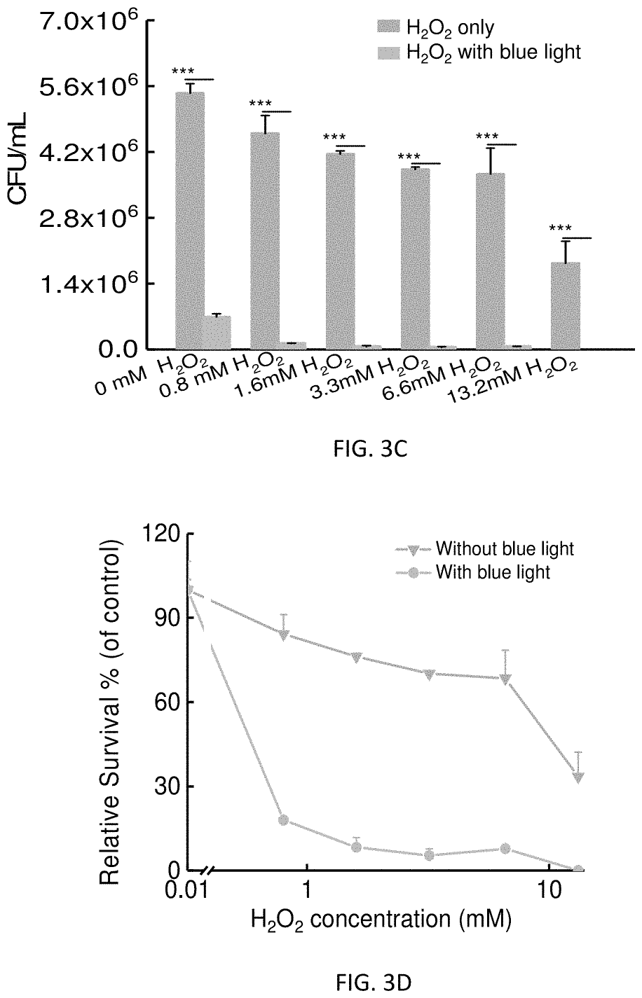

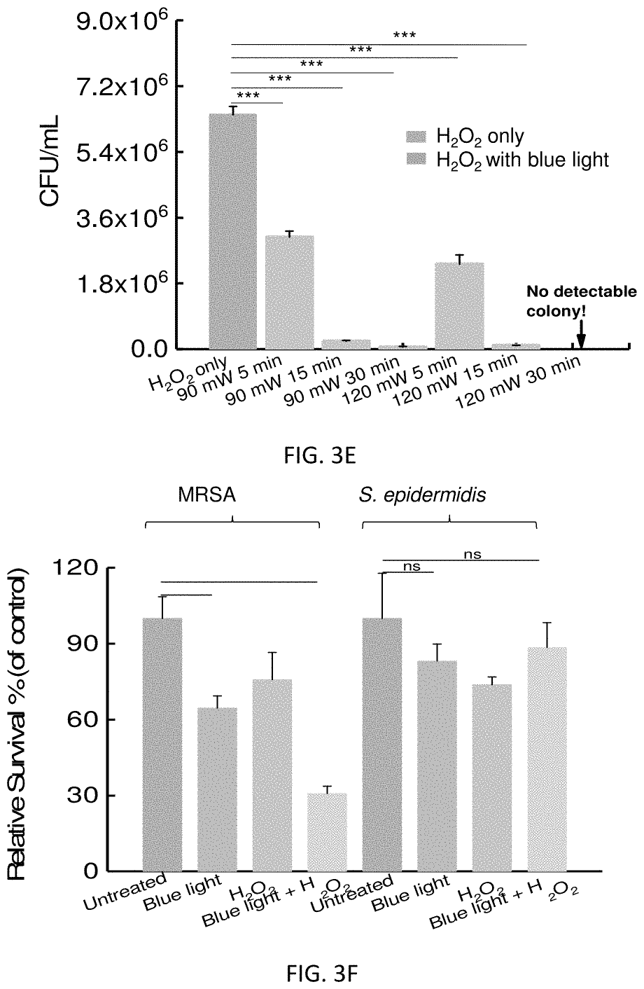

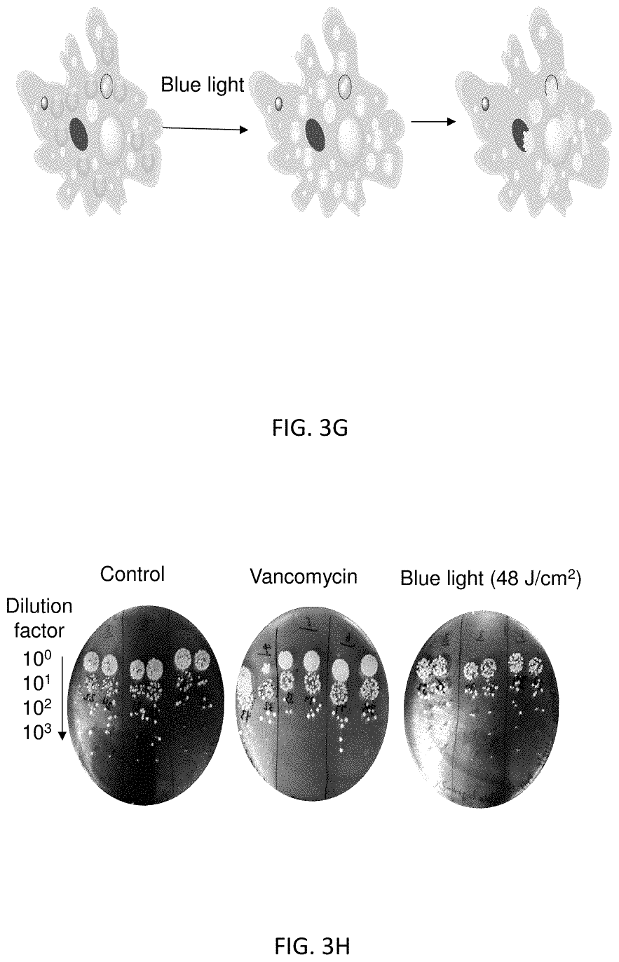

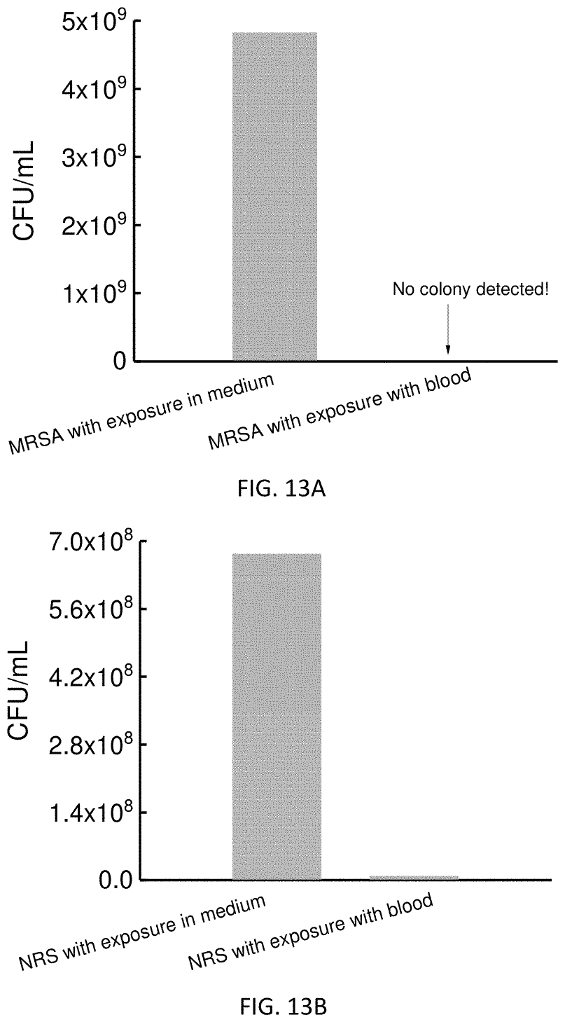

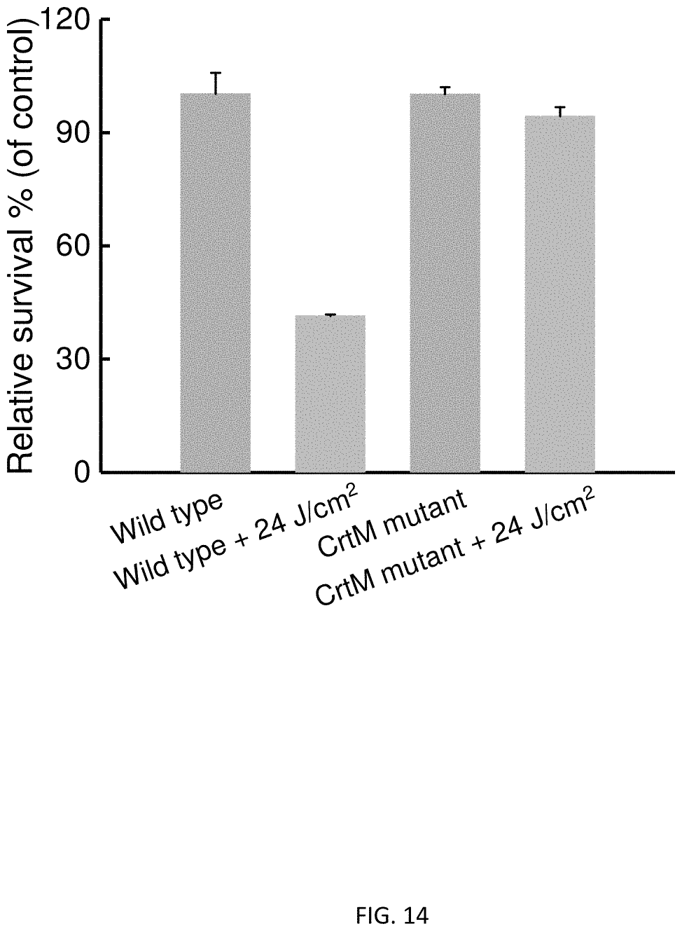

Since STX is critical to the integrity of S. aureus cell membrane (16), we asked whether blue light could eradiate MRSA through bleaching STX. It was found that increasing blue light dose could kill a growing number of MRSA (FIG. 3A), in consistence with blue-light-based bacterial killing. Moreover, we show that wild type MRSA is more sensitive to blue-light than the CrtM mutant (FIG. 13). Nevertheless, the dependence of antimicrobial effect upon blue light dose became opaque when blue light dose is higher than 216 J/cm². To investigate this reason, we carried out a real-time measurement of bacterial growth after blue light exposure. It turned out that after 10-min blue light exposure, MRSA recovered after being cultured in the medium for 30 min (FIG. 3B). Therefore, photobleaching STX alone is not sufficient to kill MRSA completely.

Because STX also serves as an indispensable antioxidant for MRSA, we then asked whether photobleaching of STX could sensitize MRSA to reactive oxygen species (ROS). We compared the survival percent of wild type MRSA after H₂O₂ treatment with or without blue light exposure. When MRSA was treated subsequently with an increasing concentration of H₂O₂ after blue light irradiance (108 J/cm²), significant reduction (p<0.001) was obtained (FIG. 3C). 13.2 mM of H₂O₂ combined with blue light exposure (108 J/cm²) eradicated all MRSA (˜10⁷, FIG. 3D). To dig out whether H₂O₂ and blue light work together as synergistically or additively, we changed the blue light dose while fixed the concentration of H₂O₂ (FIG. 3E). Combined those two effects together, a distinctive synergistic effect was found by using an established protocol (see methods). Noteworthy, this treatment does not harm benign species such as S. epidermidis (FIG. 3F) due to the lack of STX in the benign species.

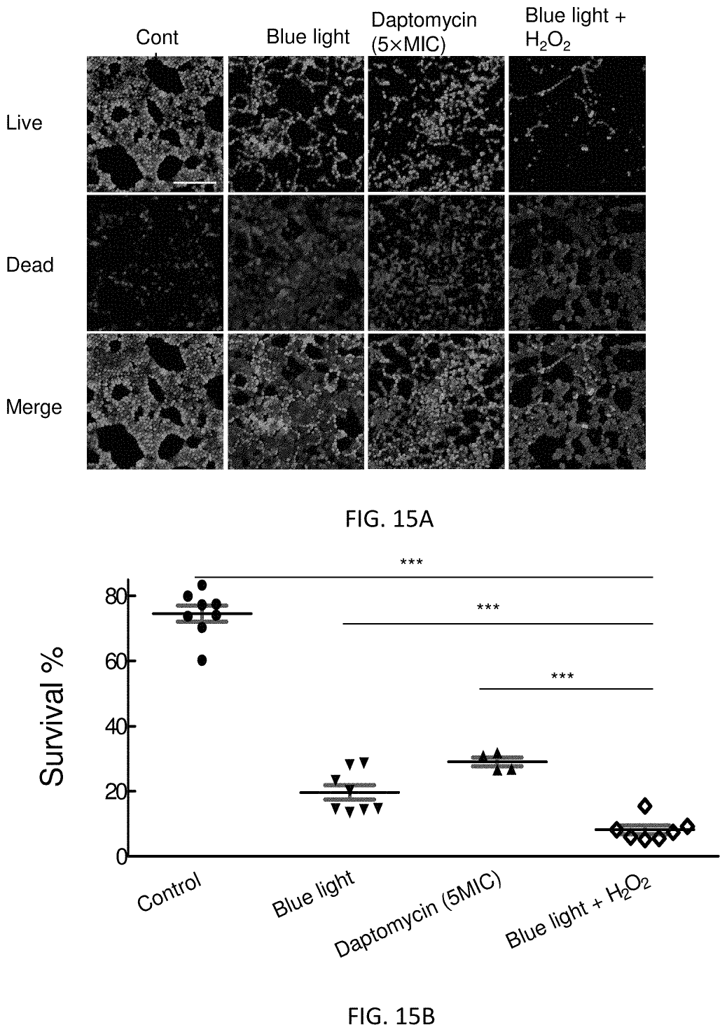

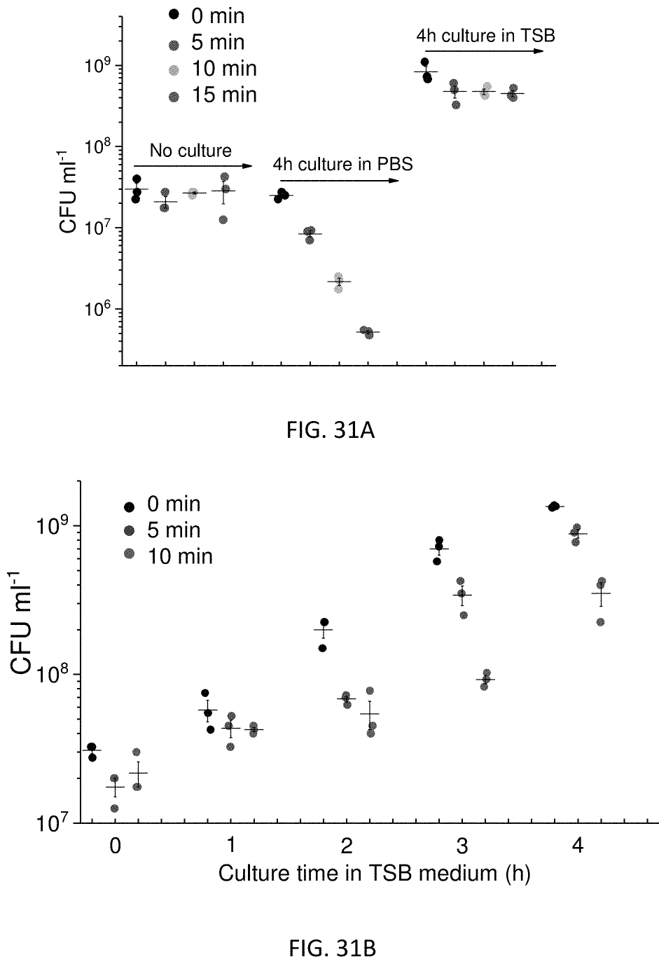

Studies dating back to at least 50 years have demonstrated that MRSA is able to invade and survive inside the mammalian cells, especially, the phagocytic cells which can't scavenge all the intracellular MRSA. Current antibiotics failed to clear the intracellular MRSA because of the difficulty in delivering drugs through the phagocytic membrane. Incomplete clearance of MRSA poses an alarming threat to the host mammalian cells. Since we have proved that blue light and H₂O₂ synergistically kill MRSA, we wondered whether blue light could synergize with intracellular ROS to eliminate MRSA inside the macrophages (FIG. 3G). We first infected the macrophage cells by incubation with MRSA 400 for 1 h. Then we applied 48 J/cm² of blue light to irradiate the macrophage cells for 2 min per each dose, two doses in total with 6-h interval between the two doses. Colony formation units (CFUs) counting was conducted (FIG. 3H). FIG. 31 compiled the statistical analysis of different groups. Noticeably, about 1-log reduction was found in the blue light-treated group in comparison with the untreated group. On the contrary, vancomycin showed no effect in killing intracellular MRSA 400. Additionally, we found that whole blood could eradicate most of MRSA after STX bleaching by blue light (FIGS. 15, A to B). These findings collectively suggest that blue light could assist neutrophils to scavenge S. aureus.

Biofilms are highly resistant to antibiotics due to their failure to penetrate the matrix of biofilm termed extracellular polymeric substances. Compared to antibiotics treatment, an unparalleled advantage of our photobleaching therapy lies in that photons can readily penetrate through a cell membrane or biofilm. To explore whether STX bleaching could eradicate MRSA inside a biofilm, we grew biofilms on the bottom of glass dish and then applied treatment on the biofilms. Blue light alone killed 80% MRSA. Blue light plus low-concentration H₂O₂ killed 92% MRSA. In contrast, application of vancomycin only killed 70% MRSA (FIGS. 15 A and B). These results suggest new opportunities of eradicating sessile bacterial cells inside biofilm that often withstand antibiotics.

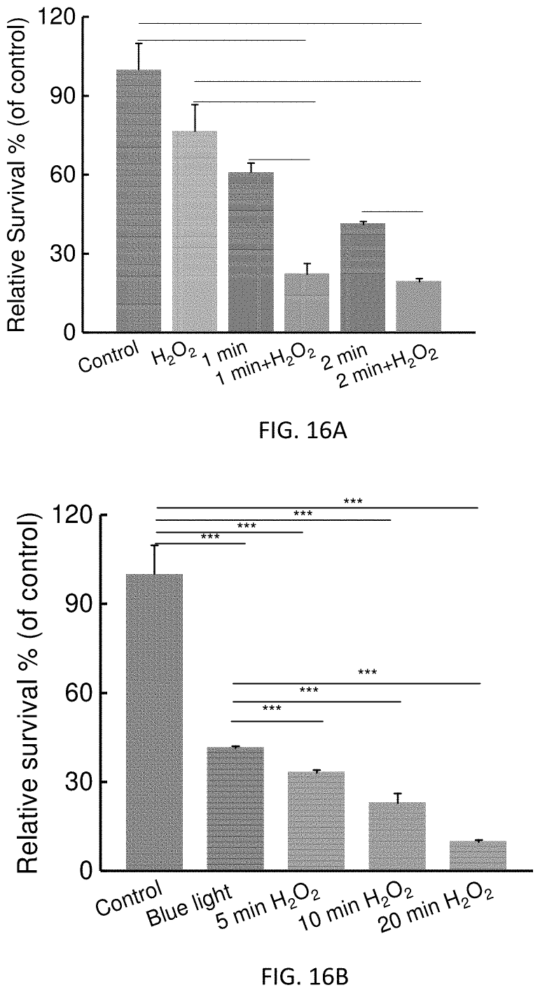

Skin infections such as diabetic foot ulceration and surgical site infections are a common cause of morbidity in the hospital and community. Notably, S. aureus accounts for 40% of skin infectious. Thus, we carried out a preclinical study to explore the potential of STX bleaching for treatment for S. aureus-induced wound infections. To facilitate the operation of in vivo experiment, we first proved that 2-min blue light exposure (24 J/cm²) could cause significant reduction of survival percent of MRSA (FIG. 16A). Two times antimicrobial efficiency was obtained when cultured with H₂O₂ (20 min, 13.2 mM) subsequently. Furthermore, 5-min culture time with H₂O₂ after 2-min blue light exposure (24 J/cm²) effectively scavenged MRSA by 60% (FIG. 16B).

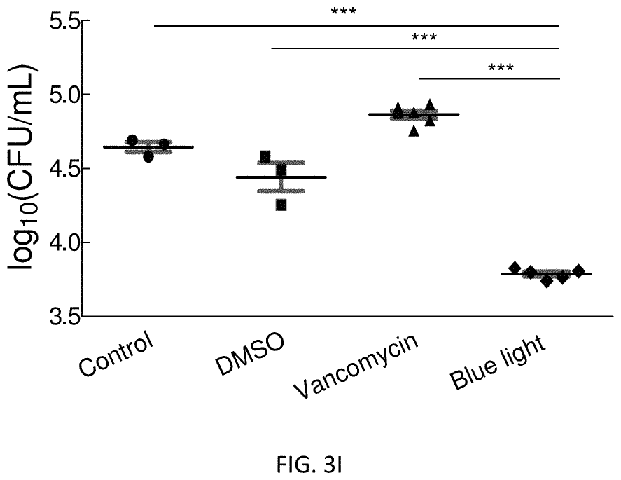

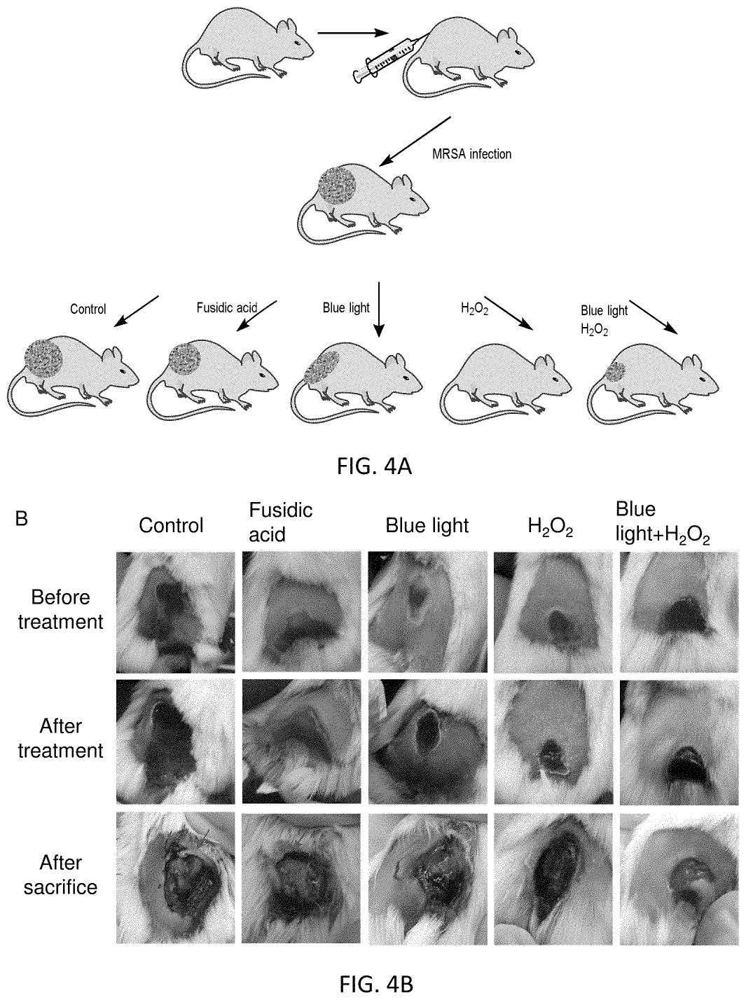

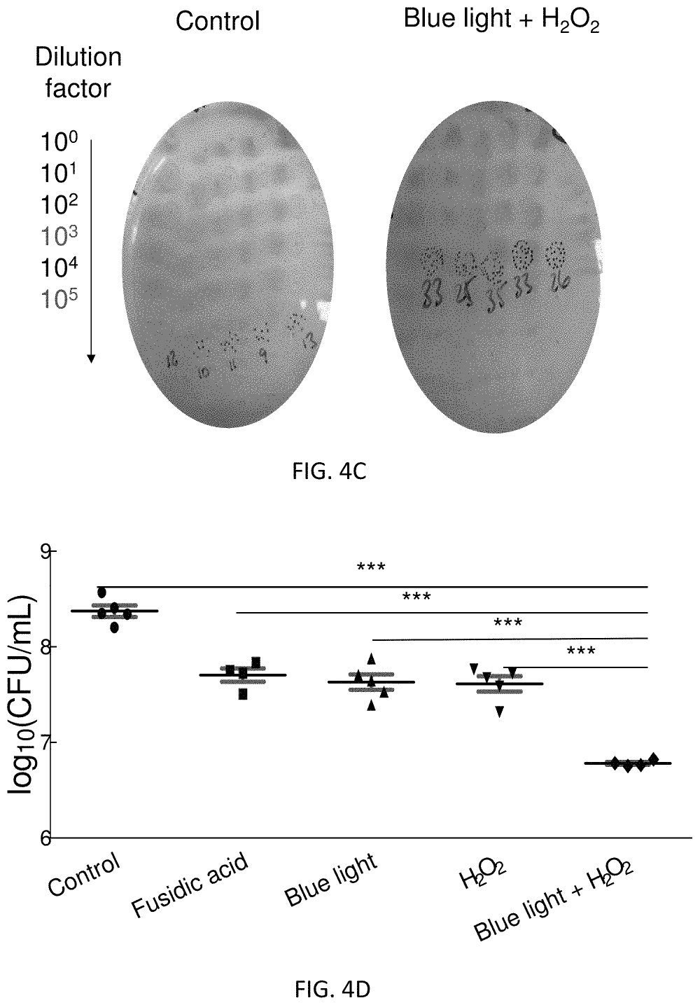

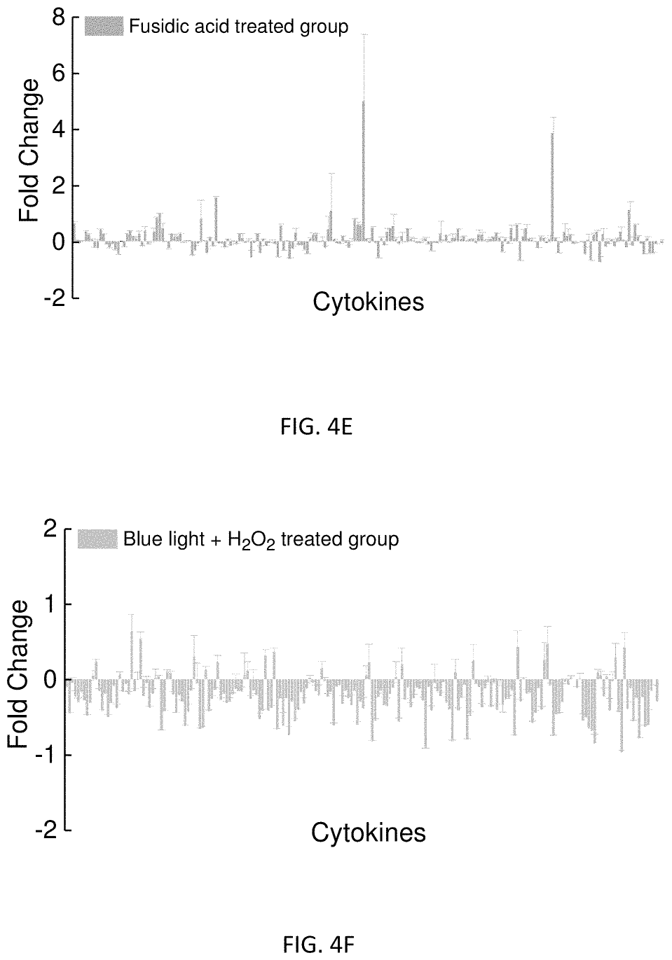

To induce MRSA-infected wound (FIG. 4A), we applied 10⁸ (in phosphate buffered solution (PBS)) of MRSA 300 to severely irritate mice skin (N=5 per group, five groups). Sixty hours post infection, an open wound formed at the site of infection (FIG. 4B (top)). Corresponding treatment was applied to each group (FIG. 4A), twice a day for three days. All treated groups demonstrated the symptom of healing, whereas the untreated group suffered from heavy infection (FIG. 4B (middle)). After sacrifice of those mice, we examined the physiological condition of the wounds. It turned out that the untreated, fusidic acid-treated and blue light-treated groups all showed the formation of pus aroused from inflammatory response of mice, whereas the H₂O₂-treated group along with blue light plus H₂O₂ treated group didn't show this sign (FIG. 4B (below)).

To quantify the antimicrobial effectiveness, we counted the number of bacteria survived inside the wound tissue by conducting CFUs study. Wound tissues were harvested into 2-mL PBS, homogenized, and then inoculated serial diluted solution onto mannitol salt agar plate (MRSA specific). The CFUs results demonstrated that blue light and H₂O₂ treated group had around 1.5-log reduction compared to the control group (FIG. 4C). Statistical analysis of CFUs from blue light and H₂O₂-treated groups depicted significant MRSA reduction compared to other groups (FIG. 4D). Noteworthy, blue light and H₂O₂-treated group has around one more log reduction than fusidic acid-treated group (FIG. 4D).

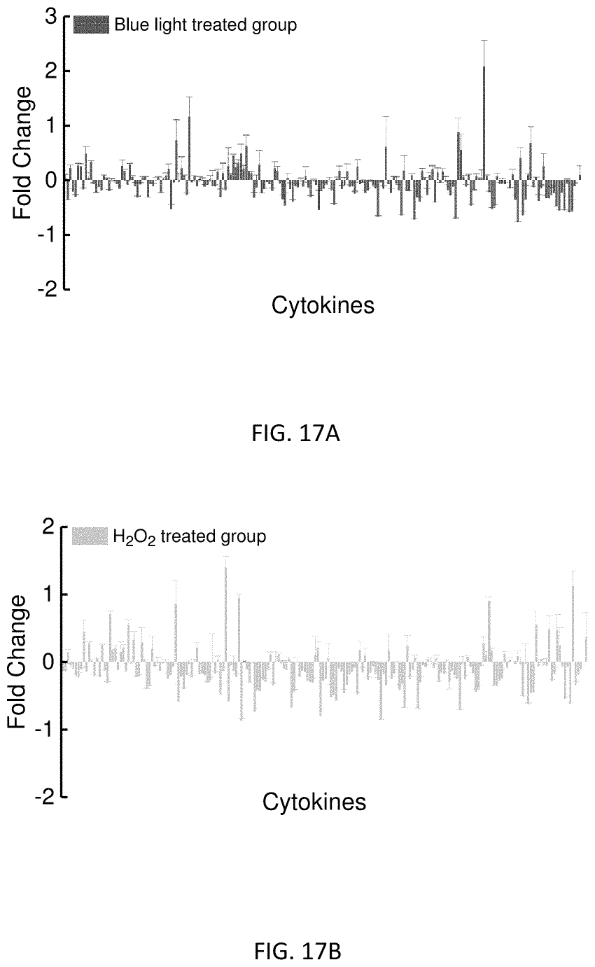

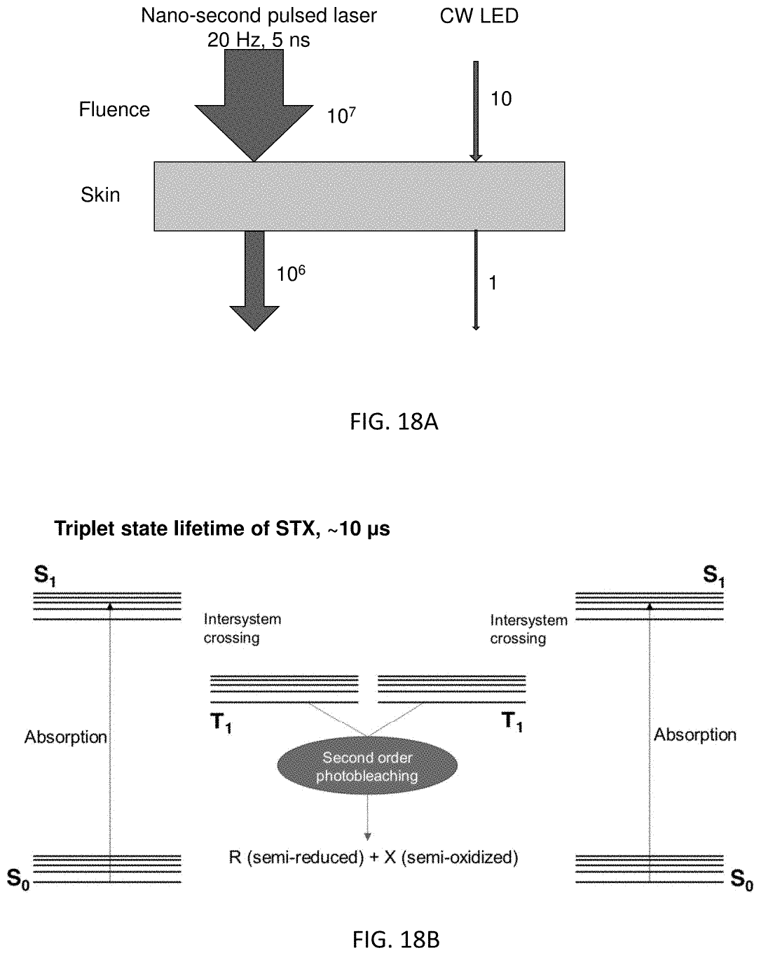

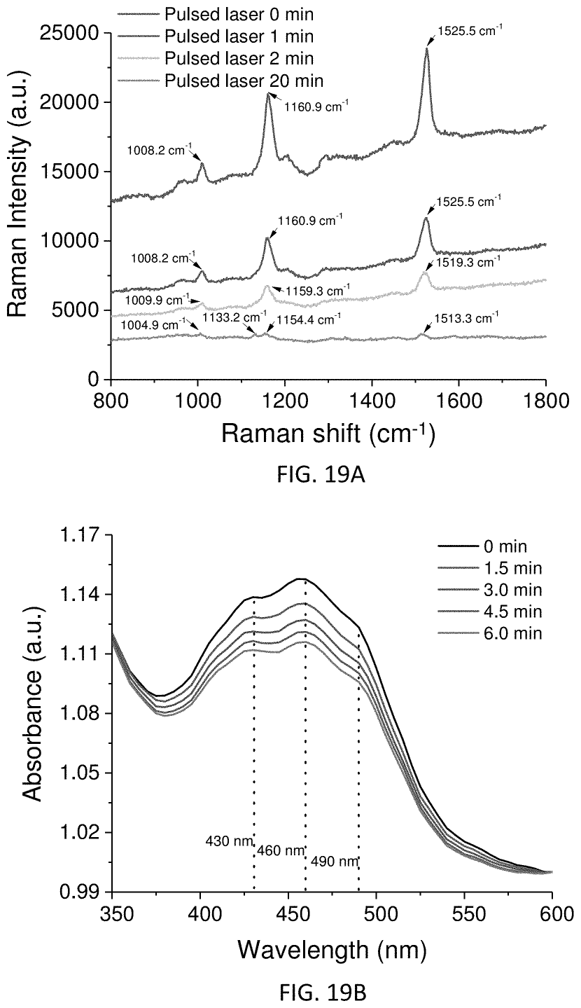

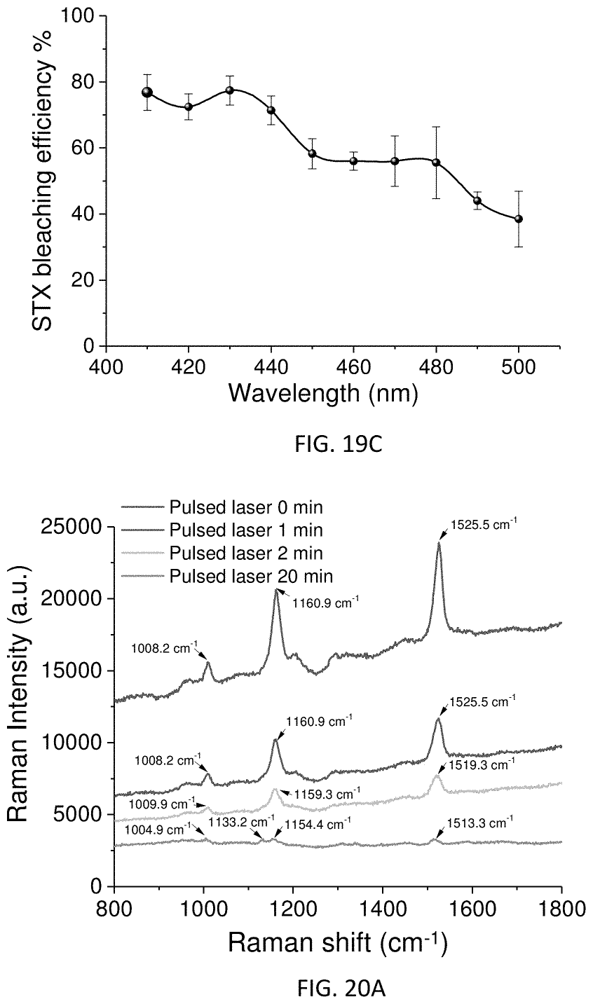

To quantify the physiological condition of the wound tissues, we measured the concentrations of 200 kinds of cytokines (Table. 1) from the supernatant of homogenized tissue solution. Cytokines are small secreted proteins released by cells and have specific effect on the interactions and communications between cells (25). Over 85% of these 200 cytokines from blue light and H₂O₂-treated group (FIG. 4F) have negative fold change, whereas around 50% of cytokines from fusidic-treated group have negative fold change (FIG. 4(E)). Moreover, compared with cytokine fold change from blue light-treated group (FIG. 17A) along with H₂O₂-treated group (FIG. 17(B)), blue light and H₂O₂-treated group exhibited the highest percent of negative fold change among those cytokines, indicating the lowest inflammatory response from wound tissue. This result solidified the synergy between blue light and H₂O₂ in treating MRSA-caused wound infections. Taken together, our findings show the exciting potential of treating drug-resistant bacteria by exploring the unique photochemistry of pigments inside the bacteria.

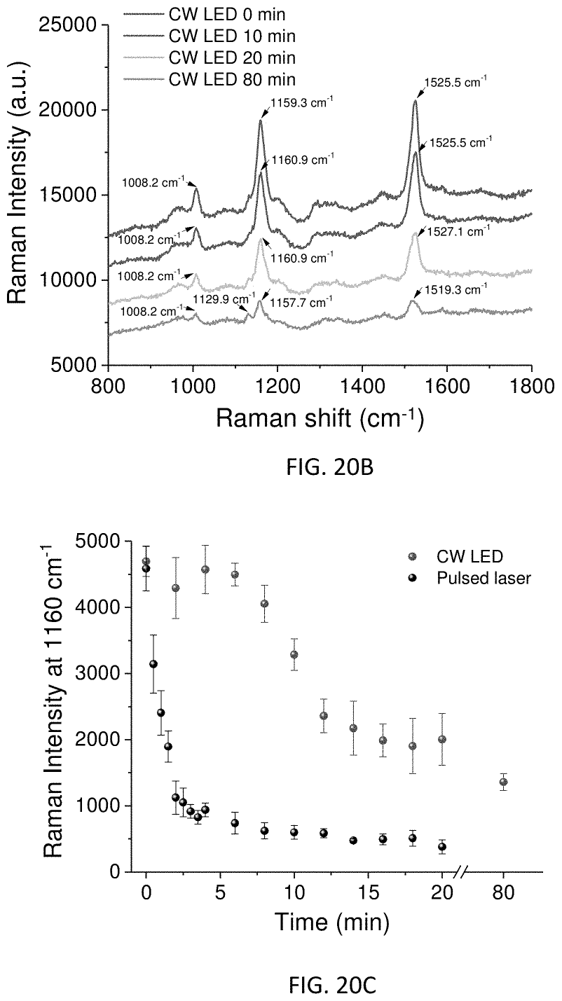

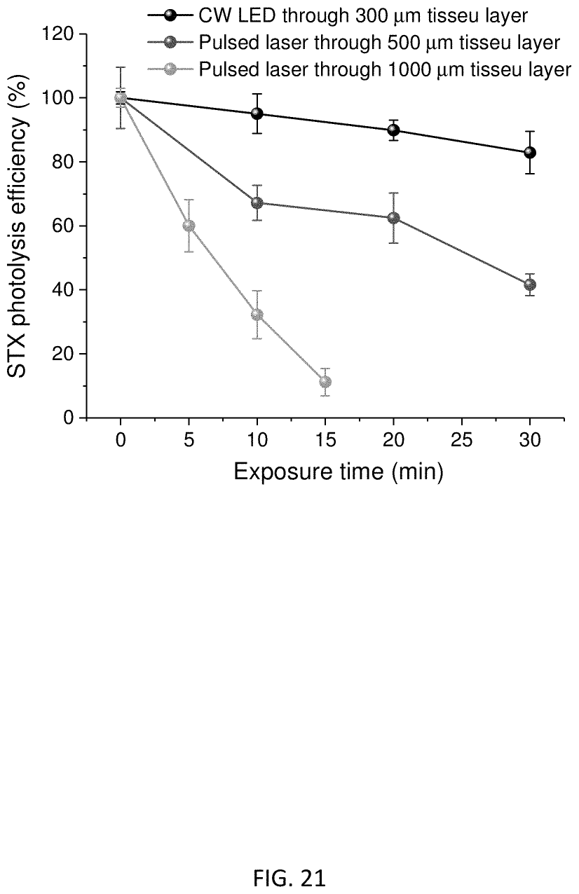

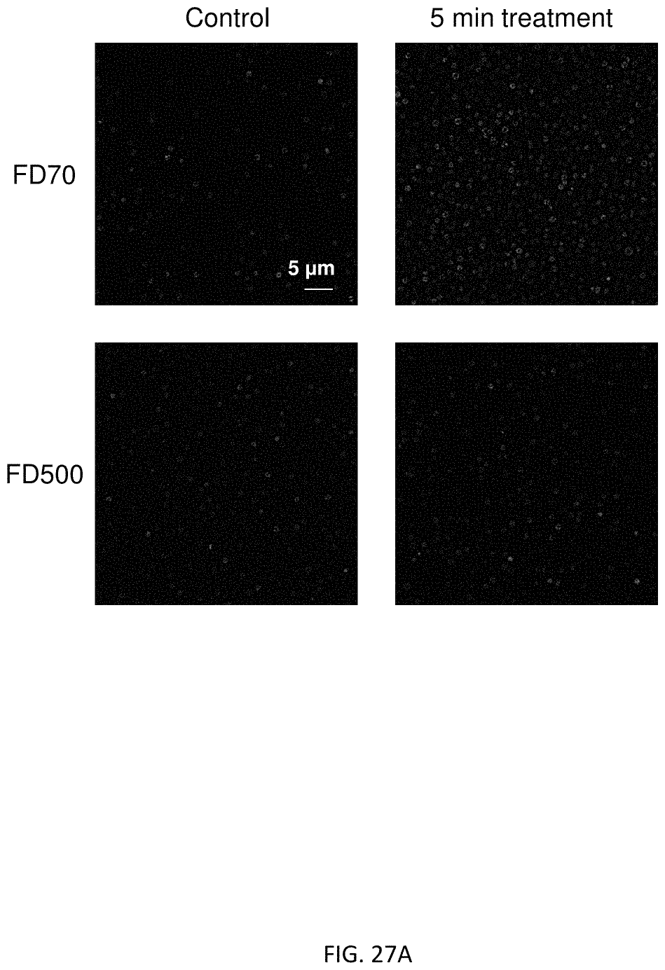

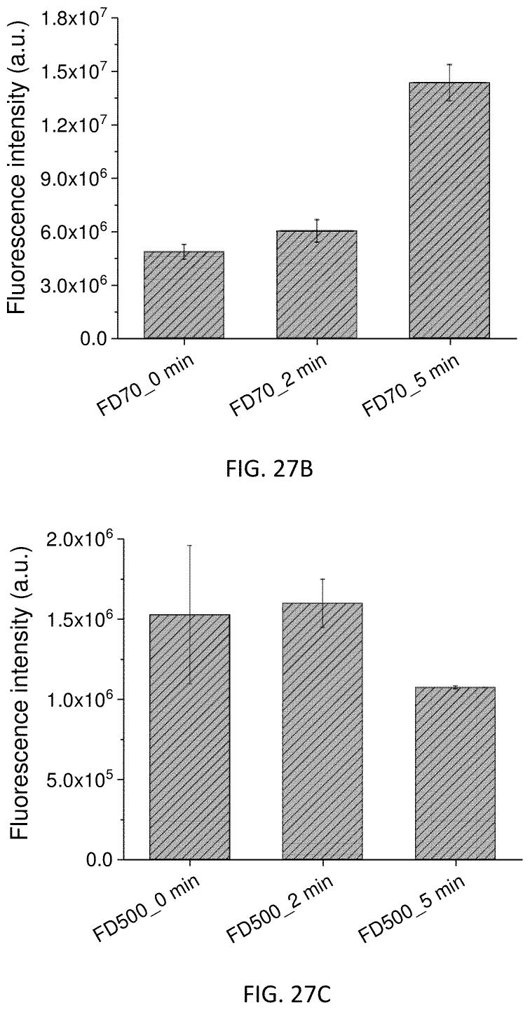

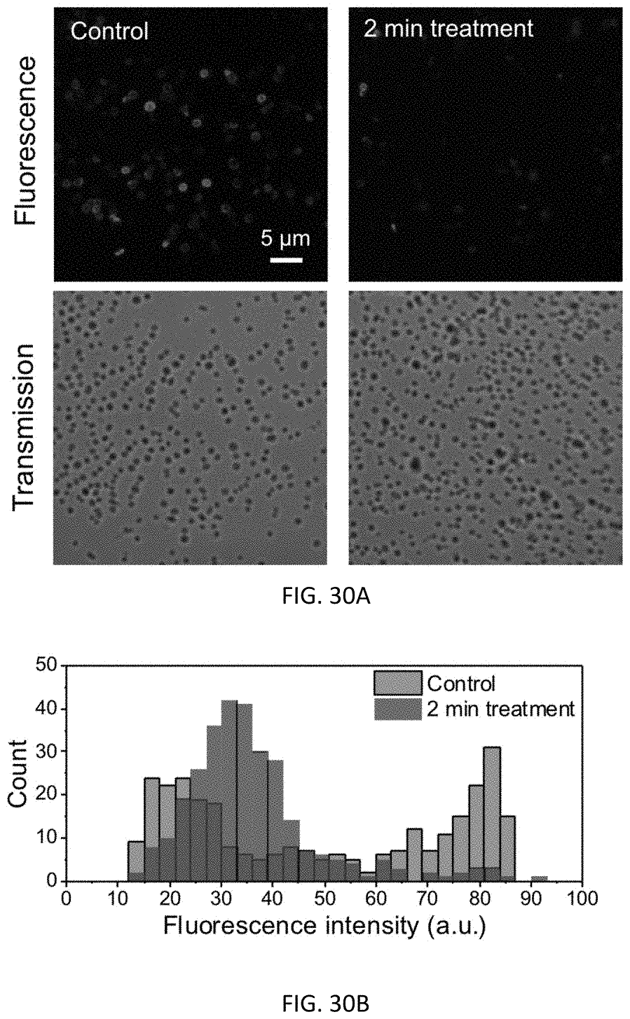

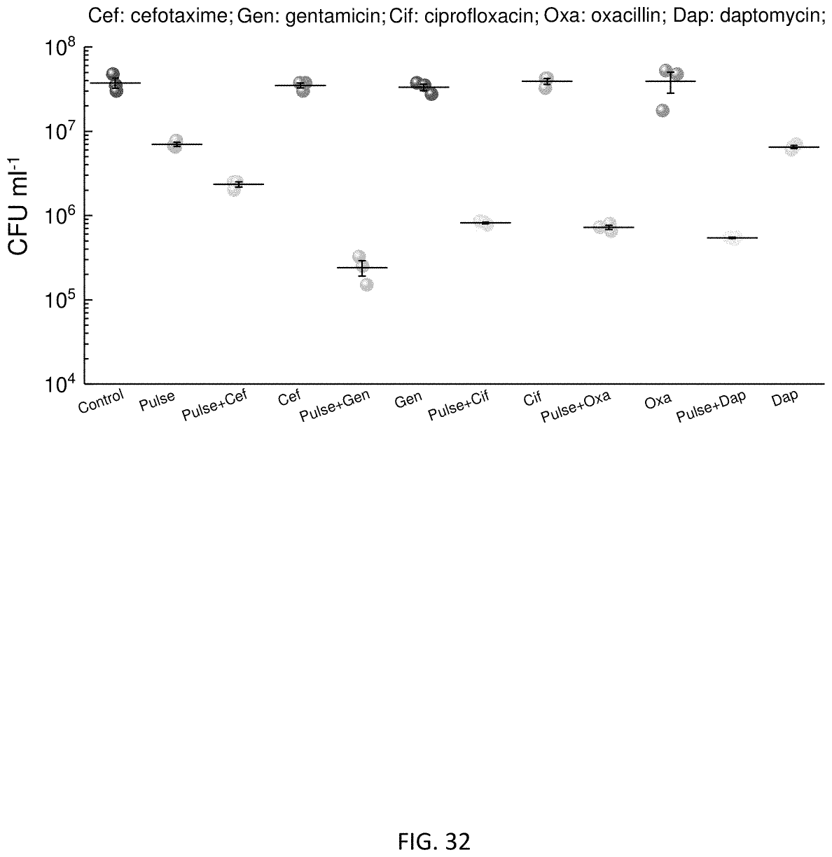

In this disclosure we have shown that high-intensity pulsed laser enable dramatically faster and deeper photolysis of staphyloxanthin. See FIGS. 20A-2C and its legend. The pulsed laser dis-assembles MRSA membrane microdomains by creating membrane pores (see FIGS. 27A, B and their legend) and unanchoring PBP2a proteins (See FIGS. 30A-30B and the legend). This photonic approach can be developed as a therapeutic platform to revive a broad spectrum of conventional antibiotics, as exemplified in FIG. 32 and its legends.

For intracellular drug delivery, it is believed that membrane permeability is a key determinant in the effectiveness of drug absorption, distribution and elimination. Selective permeability is highly dependent on molecule size and hydrophobicity due to the hydrophobic interior of bilayer lipids.

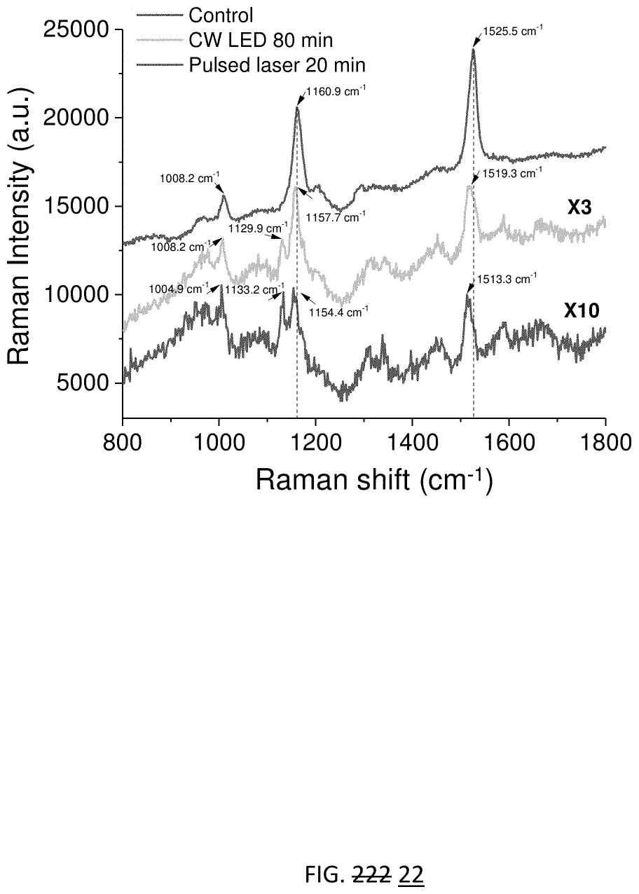

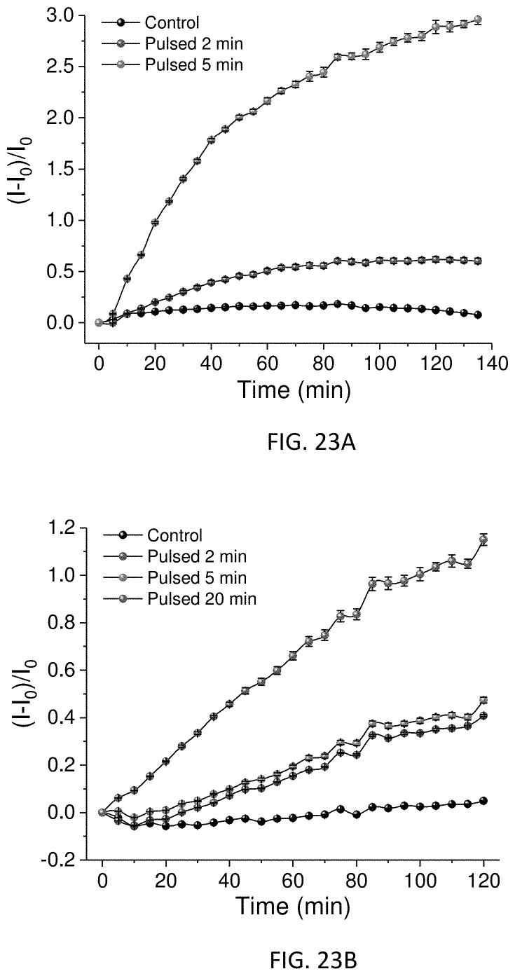

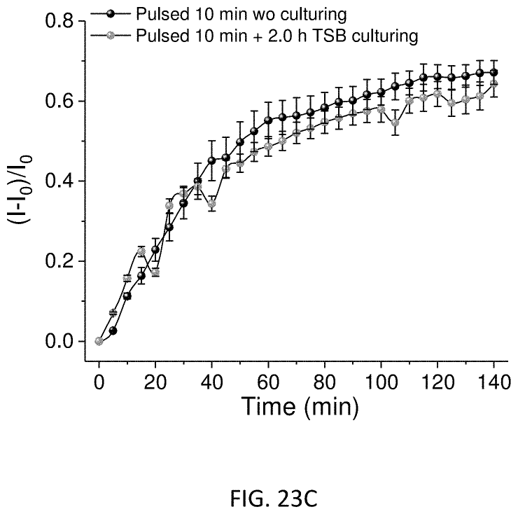

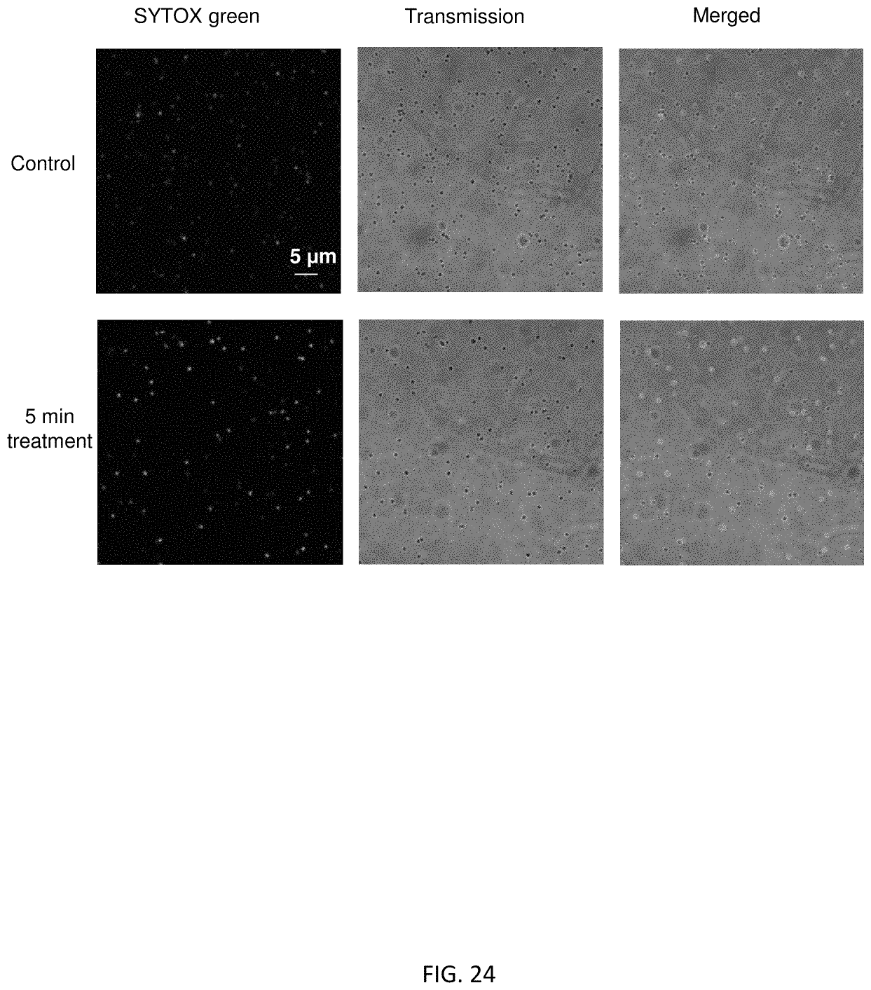

Without being confined to any theory, it is hypothesized that increased cell membrane permeability is induced by STX photolysis. This is proved by SYTOX Green study exemplified in FIGS. 23 and 24 (See their legends).

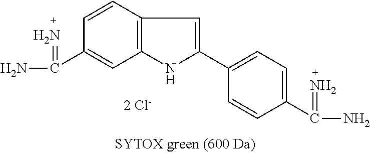 [SMILES: N=C(N)c1ccc(-c2cc3ccc(C(=N)N)cc3[nH]2)cc1]

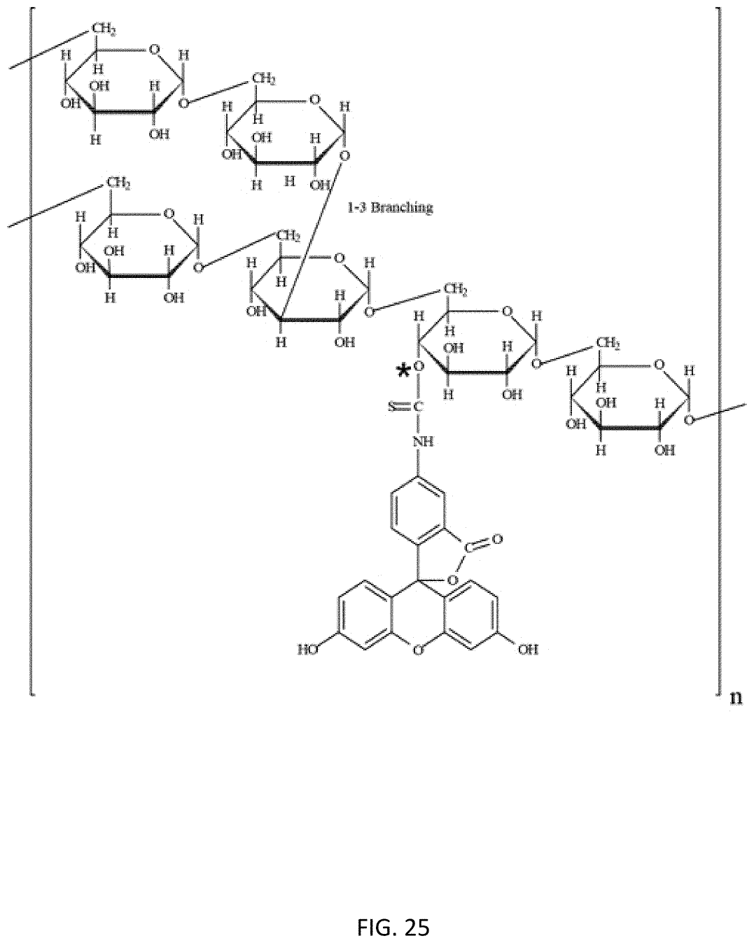

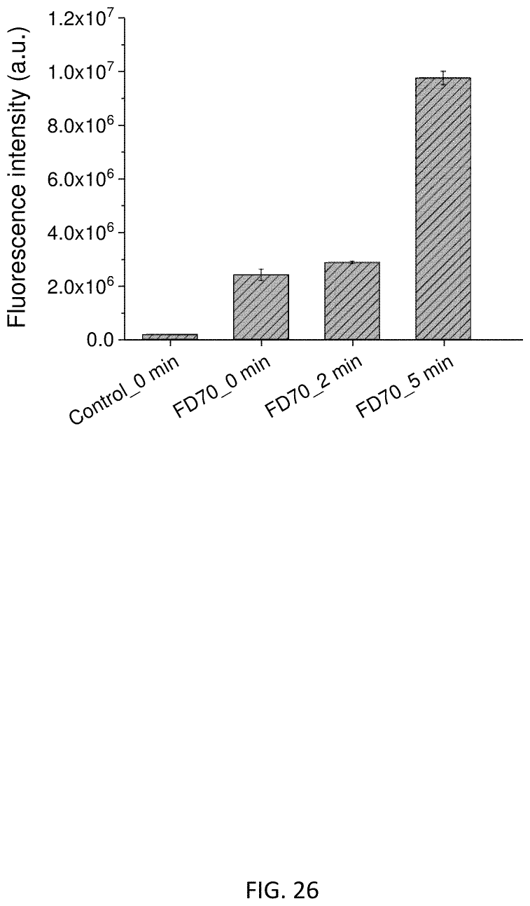

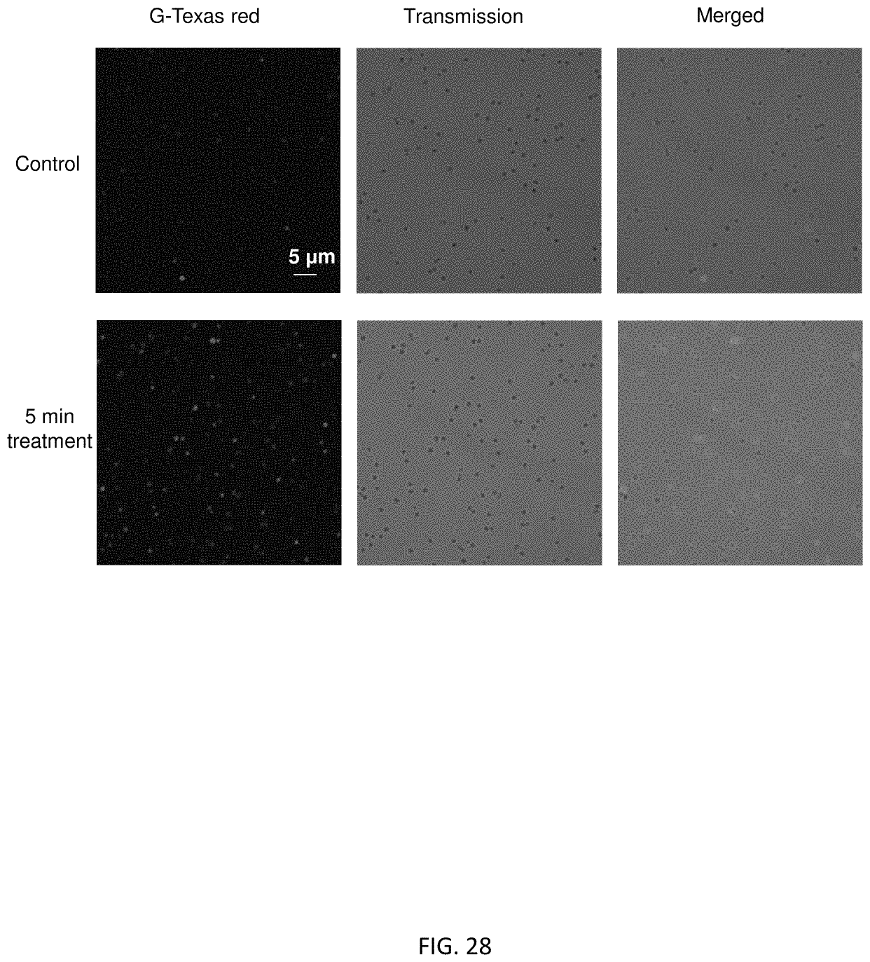

Further quantification of membrane pore size was studied by FITC-Dextran. Photolysis of staphyloxanthin created membrane poration, with pores (up to ˜10 nm level) may enable intracellular delivery of antibiotics targeting intracellular activities. See FIGS. 25, 26, 28 and their legends.

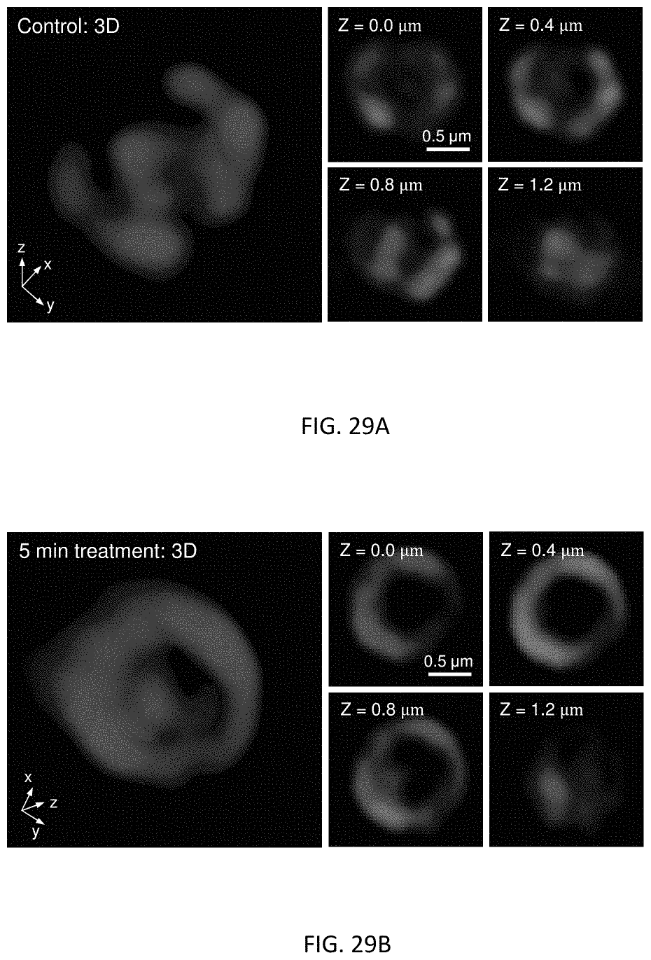

Without being limited by any theory, it is also believed that photolysis of STX disassembles functional membrane microdomains by unanchoring PBP2a proteins from membrane microdomains. FIGS. 29A-29B demonstrated a structured illumination microscopy for PBP2a accumulation within membrane microdomains and its change induced by laser treatment. This is further proved by FIGS. 30A-30B wherein preliminary western blotting results show increased amount of PBP2a in supernatant, conforming the unanchoring mechanism.

Furthermore, photo-disassembly of functional membrane microdomains also revives a broad spectrum of antibiotics against MRSA. We have shown that MRSA with compromised membrane after laser treatment is able to recover if they are put in a nutritious medium. However, significant portion of MRSA with damaged cell membrane dies if without nutritious medium. See FIGS. 31A-31B and its legend. It is worth noting that the survival percentage and recovery time of light treated MRSA depending on laser treatment time. MRSA cells need 0.5-1 hour to recover after 5 min treatment and 1-2 hour after 10 min treatment. Light treatment alone (even for a pulsed laser) is not sufficient for complete MRSA eradication. This suggests that seeking synergy with conventional antibiotics or new antibiotic drugs is a new direction of treating superbugs.

The synergy between photo-disassembly of membrane domains and conventional antibiotics is proved in FIG. 32. Nearly every class of antibiotics demonstrated synergy against MRSA. These include cetotaxine, gentamicin, ciprofloxacin, oxacillin, Gentamicin has shown more than 2 log reduction of MRSA in connection with pulsed laser treatment.

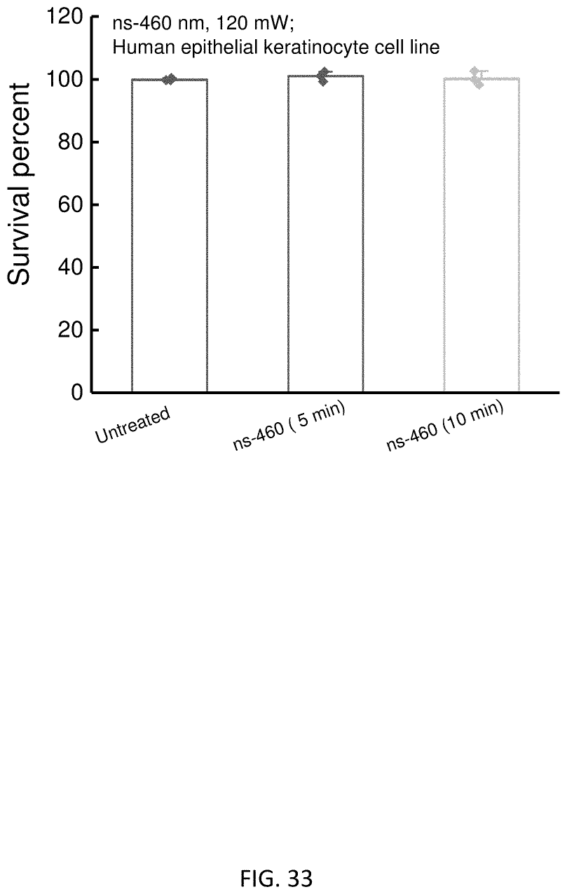

It is noted that the novel therapeutic platform has photo-selectivity on MRSA and has no photo-toxicity to human cells, as shown in FIG. 33 and its legend. Pump probe microscopy

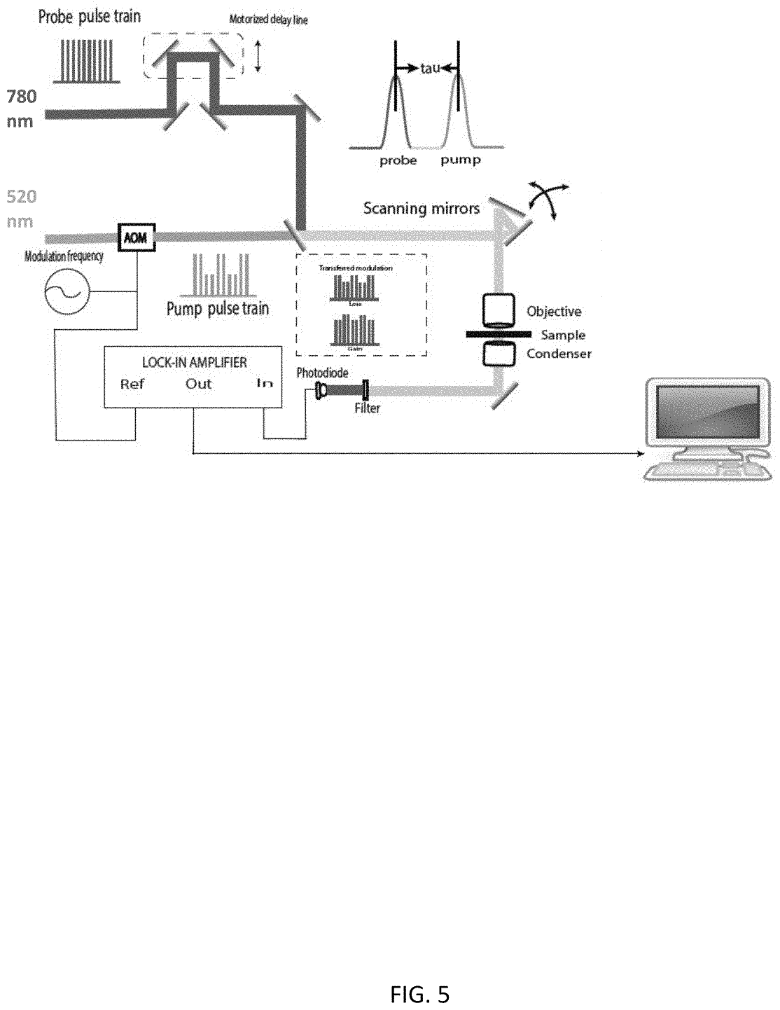

As presented in FIG. 5, an optical parametric oscillator pumped by a high-intensity mode-locked laser generates synchronous pump (520 nm) and probe (probe) pulse trains. The Ti: Sapphire oscillator is split to separate pump and probe pulse trains. Temporal delay between the pump and probe pulses is reached by guiding the pump beam through a computer-controlled delay line. Pump beam intensity is modulated with an acousto-optic modulator (AOM) and the intensity of both beams is adjusted through the combination of a half-wave plate and polarizer. Thereafter, pump and probe beams are collinearly guided into the microscope. After the interaction between the pump beam and the sample, the modulation is transferred to the un-modulated probe beam. Computer-controlled scanning galvo mirrors are used to scan the combined lasers in a raster scanning manner to create microscopic images. The transmitted light is collected by the oil condenser. Subsequently, the pump beam is spectrally filtered by an optical filter (OF) and the transmitted probe intensity is detected by a home-built photodiode (PD). A phase-sensitive lock-in amplifier then demodulates the detected signal. Therefore, pump-induced transmission changes of the sample versus time delay can be measured from the focus plane. This change over time delay shows different decay signatures from different chemicals, thus offering the origin of the chemical contrast. The real-time photobleaching process was captured and fitted by a mathematical model (derivation see supplementary text).

### Low-Level Blue Light Apparatus

As depicted in FIG. 2A, the home-built blue light LED has a major wavelength of 460 nm with full width at half maximum of 30 nm. It is comprised of three parts—a blue light LED (M470L3, Thorlabs), an adjustable collimator (COP1-A, Thorlabs), and a power controller (LEDD1B, Thorlabs). The beam spot is adjusted through the adjustable collimator (SM1P25-A, Thorlabs) depending on the size of samples to be treated. The maximal power of the blue light LED is 300 mW.

Absorbance Spectrum of Carotenoid Extract from S. aureus

The pigment extraction approach was adapted from a previous report (1). Briefly, 100 μL of bacteria solution supplemented with 1900 μL sterile Luria-Bertani (LB) broth was cultured for 24 hours with shaking (speed of 250 rpm) at 37° C. The suspension was subsequently centrifuged for two minutes at 7,000 rpm, washed once, and re-centrifuged. The pigment was extracted with 200 μL methanol at 55° C. for 20 minutes. Pigments from the CrtM mutant were extracted following the same method described above. For the treatment of S. aureus with naftifine, the protocol was adapted from a published report (2). Bacteria were cultured as described above in the presence of, 0.2 mM naftifine. The extraction procedure following the same method described above. The extracted solutions were subsequently exposed to blue light (90 mW, aperture: 1 cm×1 cm) at different time intervals (0 min, 5 min, 10 min, 20 min). Absorption spectra of the above solutions were obtained from a spectrometer (SpectraMax, M5).

### Mass Spectrometry for Photobleaching of STX

To study the photobleaching effect on STX, we extracted STX from S. aureus and exposed the extract to blue light using the procedure described above. The separation was performed on an Agilent Rapid Res 1200 high performance liquid chromatography (HPLC) system. The HPLC-MS/MS system consisted of a quaternary pump with a vacuum degasser, thermostated column compartment, auto-sampler, data acquisition card (DAD), and triple quadrupole Mass Spectrometer (QQQ) from Agilent Technologies (Palo Alto, Calif., USA). An Agilent (ZORBAX) SB-C8 column (particle size: 3.5 μm, length: 50 mm, and internal diameter: 4.6 mm) was used at a flow rate of 0.8 mL/min. The mobile phase A was water with 0.1% formic acid and mobile phase B was acetonitrile with 0.1% formic acid. The gradient increased linearly as follows: 5% B, from one to five min; 95% B from five to six min, and 5% B. Column re-equilibration was 6-10 min, 5% B. The relative concentration of STX was quantified using MS/MS utilizing the Agilent 6460 Triple Quadrupole mass spectrometer with positive electrospray ionization (ESI). Quantitation was based on multiple reaction monitoring. Mass spectra were acquired simultaneously using electrospray ionization in the positive modes over the range of m/z 100 to 1000. Nitrogen was used as the drying gas flow.

In order to understand how STX degrades when exposed to blue light, an Agilent 6545 Q-TOF (Agilent, Santa Clara, Calif., USA) was exploited to conduct the separation and quantification steps. This ultra-performance liquid chromatography (UPLC)-MS/MS utilized an Agilent (ZORBAX) SB-C8 column (particle size: 3.5 μm, length: 50 mm, and internal diameter: 4.6 mm) to conduct the separation at a flow rate of 0.8 mL/min. The relative concentration of STX was quantified using MS/MS utilizing the Agilent 6545 quadrupole time of flight (Q-TOF) MS/MS with positive ESI. The mobile phase was composed of water (A) and acetonitrile (B). The gradient solution with a flow rate of 0.8 mL/min was performed as follows: 85% B, from 0 to 30 min; 95% B, from 30 to 31 min; 85% B, from 31 to 35 min; 85% B, after 35 min. The sample injection volume was 20 μL. The UPLC-MS/MS analysis was performed in positive ion modes in the range of m/z 100-1100.

In Vitro Assessment of Synergy Between Blue Light and H₂O₂

MRSA USA300 was cultured in sterile LB broth in a 37° C. incubator with shaking (at 250 rpm) until the suspension reached the logarithmic growth phase (OD₆₀₀=0.6). Thereafter, an aliquot (20 μL) of the bacterial suspension was transferred onto a glass slide. Samples were exposed to blue light at different time-lengths and variable light intensities. For groups treated with hydrogen peroxide, bacteria were collected in either LB or phosphate-buffered saline (PBS) supplemented with hydrogen peroxide at different concentrations (0 mM, 0.8 mM, 1.6 mM, 3.3 mM, 6.6 mM, and 13.2 mM). The solutions were cultured for 20 min. The solution was serially diluted in sterile PBS and transferred to LB plates in order to enumerate the viable number of MRSA colony-forming units (CFUs). Plates were incubated at 37° C. for 24 hours before counting viable CFU/mL. Data are presented as viable MRSA CFU/mL and percent survival of MRSA CFU/mL in the treated groups. The data was analyzed via a two-paired t-test (OriginPro 2017). Synergistic effect was confirmed by an equation (see supplementary text).

Fluorescence Mapping of Live/Dead S. aureus in Biofilm

An overnight culture of S. aureus (ATCC 6538) was grown in a 37° C. incubator with shaking (at 250 rpm). Poly-D-lysine (Sigma Aldrich) was applied to coat the surface of glass bottom dishes (35 mm, In Vitro Scientific) overnight. The overnight culture of S. aureus was diluted (1:100) in LB containing 5% glucose and transferred to the glass bottom dishes. The plates were incubated at 37° C. for 24-48 hours in order to form mature biofilm. Thereafter, the media was removed the surface of the dish was washed with sterile water to remove planktonic bacteria. Plates were subsequently treated with blue light alone (200 mW/cm², 30 min), hydrogen peroxide (13.2 mM, 20 minutes) alone, or a combination of both. Groups receiving H₂O₂ were quenched through addition of 0.5 mg/mL catalase (Sigma Aldrich, 50 mM, pH=7 in potassium buffered solution). After treatment, biofilms were immediately stained with fluorescence dyes, as follows.

To confirm the existence of biofilm on the glass bottom surface, a biofilm matrix stain (SYPRO® Ruby Biofilm Matrix Stain, Invitrogen) was utilized. Biofilms were stained with the LIVE/DEAD biofilm viability kit (Invitrogen) for 30 minutes. The biofilms were washed with sterile water twice and then imaged using a fluorescence microscope (OLYMPUS BX51, objective: 60×, oil immersion, NA=1.5). Two different excitation channels (Live: FITC, Dead: Texas Red) were utilized in order to map the ratio of live versus dead cells within the biofilm. The acquired images were analyzed by ImageJ. Statistical analysis was conducted via a two-paired t-test through GraphPad Prism 6.0 (GraphPad Software, La Jolla, Calif.).

### Intracellular MRSA Infection Model

Murine macrophage cells (J774) were cultured in Dulbecco's Modified Eagle Medium (DMEM) supplemented with 10% fetal bovine serum (FBS) at 37° C. with CO₂ (5%). Cells were exposed to MRSA USA400 at a multiplicity of infection of approximately 100:1. 1-hpost-infection, J774 cells were washed with gentamicin (50 μg/mL, for one hour) to kill extracellular MRSA. Vancomycin, at a concentration equal to 2 μg/mL (4×minimum inhibitory concentration (MIC)), was added to six wells. Six wells received blue light treatment twice (six hours between treatments) for two minutes prior to addition of DMEM+10% FBS. Three wells were left untreated (medium+FBS) and three wells received dimethyl sulfoxide at a volume equal to vancomycin-treated wells. Twelve hours after the second blue light treatment, the test agents were removed; J774 cells were washed with gentamicin (50 μg/mL) and subsequently lysed using 0.1% Triton-X 100. The solution was serially diluted in phosphate-buffered saline and transferred to Tryptic soy agar plates in order to enumerate the MRSA colony-forming units (CFU) present inside infected J774 cells. Plates were incubated at 37° C. for 22 hours before counting viable CFU/mL. Data are presented as log₁₀(MRSA CFU/mL) in infected J774 cells in relation to the untreated control. The data was analyzed via a two-paired t-test, utilizing GraphPad Prism 6.0 (GraphPad Software, La Jolla, Calif.).

### In Vivo MRSA Mice Wound Model

To initiate the formation of a skin wound, five groups (n=5) of eight-week old female Balb/c mice (obtained from Harlan Laboratories, Indianapolis, Ind., USA) were disinfected with ethanol (70%) and shaved on the middle of the back (approximately a one-inch by one-inch square region around the injection site) one day prior to infection as described from a reported procedure (3). To prepare the bacterial inoculum, an aliquot of overnight culture of MRSA USA300 was transferred to fresh Tryptic soy broth and shaken at 37° C. until an OD₆₀₀ value of ˜1.0 was achieved. The cells were centrifuged, washed once with PBS, re-centrifuged, and then re-suspended in PBS. Mice subsequently received an intradermal injection (40 μL) containing 2.40×10⁹ CFU/mL MRSA USA300. An open wound formed at the site of injection for each mouse, ˜60 hrs post-infection.

Topical treatment was initiated subsequently with each group of mice receiving the following: fusidic acid (2%, using petroleum jelly as the vehicle), 13.2 mM H₂O₂ (0.045%, two-minute exposure), blue light (two-minute exposure, 24 J/cm²), or a combination of blue light (two-minute exposure)+13.2 mM H₂O₂ (two-minute exposure). One group of mice was left untreated (negative control). Each group of mice receiving a particular treatment regimen was housed separately in a ventilated cage with appropriate bedding, food, and water. Mice were checked twice daily during infection and treatment to ensure no adverse reactions were observed. Mice were treated twice daily (once every 12 hours) for three days, before they were humanely euthanized via CO₂ asphyxiation 12 hours after the last dose was administered. The region around the skin wound was lightly swabbed with ethanol (70%) and excised. The tissue was subsequently homogenized in PBS. The homogenized tissue was then serially diluted in PBS before plating onto mannitol salt agar plates. Plates were incubated for at least 19 hours at 37° C. before viable MRSA CFU/mL were counted for each group. Outlier was removed based upon the Dixon Q Test. Data were analyzed via a two-paired t-test, utilizing GraphPad Prism 6.0 (GraphPad Software, La Jolla, Calif.).

### Statistical Analysis

Data are means (black) with standard error of mean (red). Statistical analysis was conducted through two-paired t-test. *** means significantly different with the p-value<0.001. ** means significantly different with the p-value<0.01. * means significantly different with the p-value<0.05.

### Supplementary Text

### Mathematical Model to Fit the Photobleaching Process Captured by Real-Time Transient Absorption Microscopy

Here, we utilized a mathematical model which was originally used to depict the photobleaching of photosensitizers happening during the photodynamic process (4):

$$\frac{d [ C ]}{dt} = - {k}_{1} [ C ] [ R ] , \qquad ( 1 )$$

where t is the duration time, [C] is the concentration of chromophore (carotenoids for S. aureus), k₁(k₁=1/τ₁) is the rate constant of first-order photobleaching which τ₁ is the first order photobleaching time and [R] is the concentration of active agents (the chromophores which have interaction with light), here:

[R]˜[R]ₒ+k₂[C] (2)

, where k₂ (k₂=/τ₂) is the rate constant of second-order photobleaching which r₂ is the second order photobleaching time, [R]₀ is the original concentration of active agent, respectively. Combined equation (1) and equation (2) together,

$$\frac{d [ C ]}{dt} = - \frac{1}{{τ}_{1}} * [ C ] - \frac{1}{{τ}_{2} * {[ C ]}_{0}} * {[ C ]}^{2} \qquad ( 3 )$$

the solution for equation (3) is:

$$\frac{{[ C ]}_{t}}{{[ C ]}_{0}} = A * \frac{exp ( - \frac{t}{{τ}_{1}} )}{1 + \frac{{τ}_{1}}{{τ}_{2}} * ( 1 - exp ( - \frac{t}{{τ}_{1}} )} , \qquad ( 4 )$$

where A is a constant. When first order photobleaching process pivots (usually happening for low concentration of chromophore and the involvement of oxygen), τ₂→∞, equation (4) becomes:

$$\frac{{[ C ]}_{t}}{{[ C ]}_{0}} = A * exp ( - \frac{t}{{τ}_{1}} ) , \qquad ( 5 )$$

which is similar to first-order kinetic reaction. At this occasion, the photobleaching rate is proportional linearly to the concentration of chromophore. When second order photobleaching process dominates (usually happening for high concentration of chromophore, triplet-triplet annihilation), τ₁→∞, equation (4) becomes:

$$\frac{{[ C ]}_{t}}{{[ C ]}_{0}} = A * \frac{1}{1 + \frac{t}{{τ}_{2}}} , \qquad ( 6 )$$

under this condition, the photobleaching rate is proportional to the square of concentration of chromophore. According to the fitting result, S. aureus belongs to second order bleaching with τ₁→∞.

Equation to determine synergistic antimicrobial effect

The synergistic effect between blue light and H₂O₂ was determined by the combination assay as described previously [X]. The fractional inhibitory concentration (FIC) index was calculated as follows: FIC of drug A=MIC of drug A in combination/MIC of drug A alone, FIC of drug B=MIC of drug B in combination/MIC of drug B alone, and FIC index=FIC of drug A+FIC of drug B. An FIC index of ≤0.5 is considered to demonstrate synergy. Additive was defined as an FIC index of 1. Antagonism was defined as an FIC index of >4. According to estimation, in the case of blue light and H₂O₂, the FIC is ≤0.38<0.5, thus, blue light exerts synergistic antimicrobial effect with H₂O₂ to eradicate MRSA.

Current antimicrobial development pipeline has failed to meet the growing needs of new and effective antibiotics to fight bacterial infections. Here, we demonstrate an unconventional phototherapy approach to combat MRSA antibiotic resistance by targeting its STX virulence factor. This approach fundamentally relies on the interaction between photons and its endogenous chromophores. Despite the notion exists for decades, the underlying mechanism of blue light antimicrobial effect is still a mystery and its treatment efficacy is limited, hampering its clinical applications. Here, we identify STX as the molecular target of photons and subject to photolysis in the entire blue range. This finding directly challenges the traditionally well accepted hypothesis of blue light-sensitive endogenous porphyrins, meanwhile, profoundly opens new opportunities in this field. The detailed study of STX photochemistry and its photolysis kinetics further suggest a short-pulsed laser to nonlinearly accelerate STX photolysis efficiency, speed, and depth that are beyond the reach of low-level light sources.

We further show that STX photolysis disorganizes and malfunctions membrane for antibiotic defense in three distinct aspects. First, the disruption renders membrane permeable to antibiotic that target intracellular activities e.g. fluoroquinolones and aminoglycosides. Second, membrane becomes more fluid that facilitates the membrane insertion of membrane targeting antibiotic, e.g. daptomycin. Third, proteins, e.g. PBP2a, that anchors within in the FMM is detached and malfunctioned to defend penicillin. These membrane damage mechanisms demonstrate a novel approach to revive a broad spectrum of conventional antibiotic to combat MRSA. Noteworthy, this approach is fundamentally different from photodynamic therapy, as it relies on endogenous STX to disrupt cell membrane, thus specifically targeting S. aureus, instead of using externally administrated photosensitizer-induced ROS for unselective bacterial eradication.

STX-targeted phototherapy has shown promising potential as a novel treatment platform. Future studies can examine synergies with other classes of antibiotics, as well as the host innate immune system, and/or other reactive oxygen species. For example, disassembly of FMM could be further extended to revive chloramphenicol, as its resistance primarily due to the overexpression of norA-encoded multidrug-resistance efflux pumps within the microdomains³⁵. As STX has the antioxidant function to shield MRSA from attacks by ROS, effective STX photolysis could further render MRSA susceptible to oxidative host killing including macrophage cells and neutrophils′⁷. Similar to daptomycin, the modulation on cell membrane fluidity via laser treatment can facilitate non-oxidative host defense of cationic antimicrobial peptides²⁵. Moreover, this platform can be further exploited to screen lead compounds, particularly for those with intracellular targets.

Targeting MRSA STX virulence by photons exemplifies the approach that utilizes the photochemistry between photons and endogenous chromophores to develop a phototherapy platform for bacterial infections. Carotenoids that has structural and functional similarity broadly present in many other bacterial and fungal species, thus can be photochemically decomposed or modulated in a similar manner. Notably, pigmentation is a hallmark for many pathogenic microbes; these pigments similarly promote microbial virulence and exhibits pro-inflammatory or cytotoxic properties. Therefore, these pigments could be the targets of photons via either photochemistry or photothermal approach. Several bacterial enzymes that regulate their virulence are also found sensitive to photons. Therefore, phototherapy approaches based on these specific photon-chromophore interactions could be further explored along this direction.

### Example 1. Pulsed Blue Laser Photolysis of Staphyloxanthin

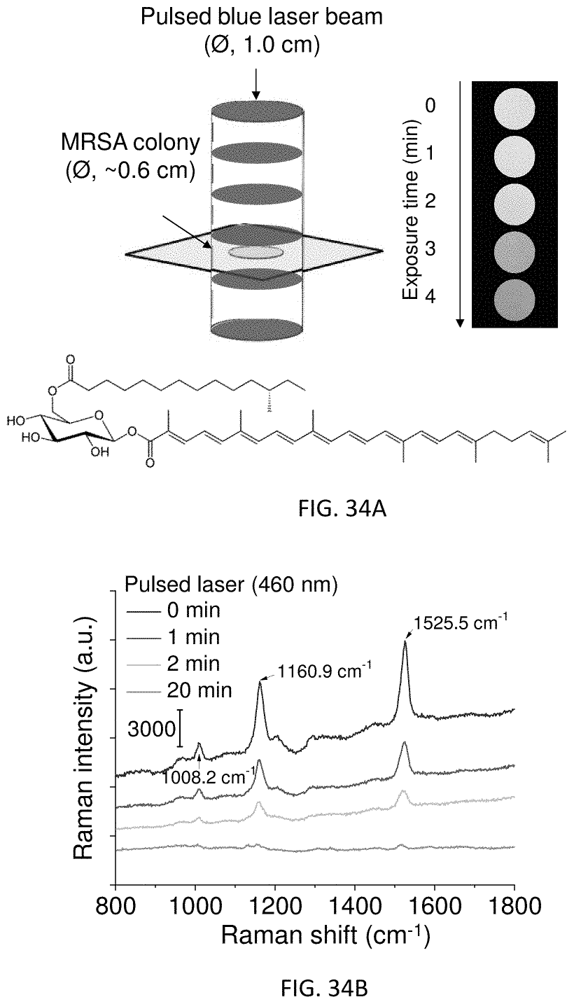

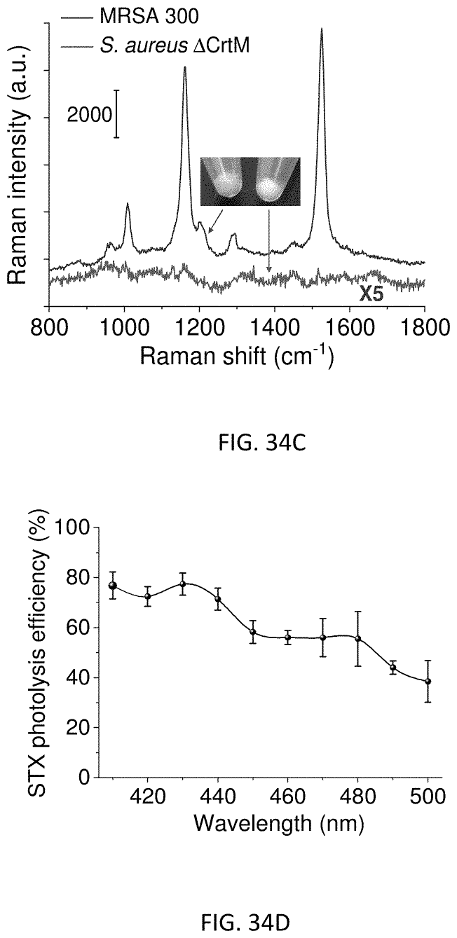

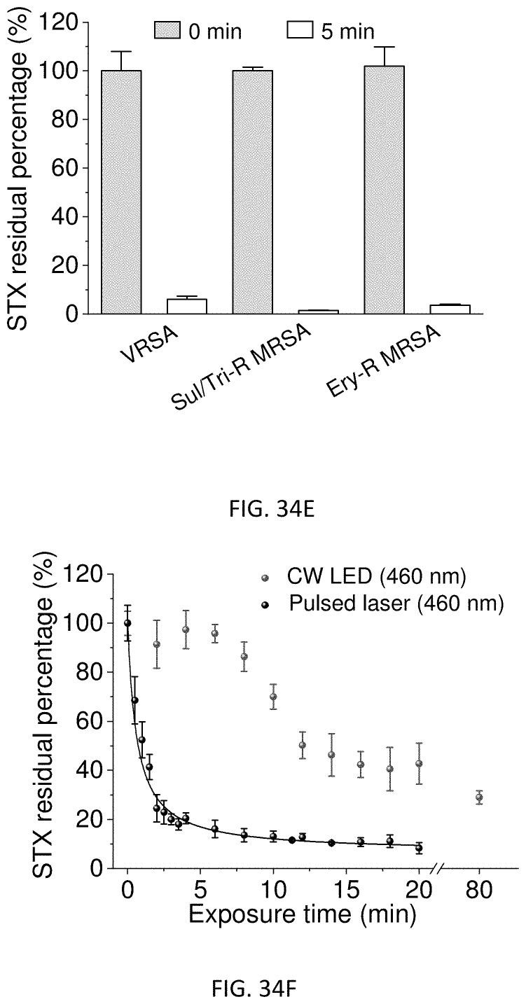

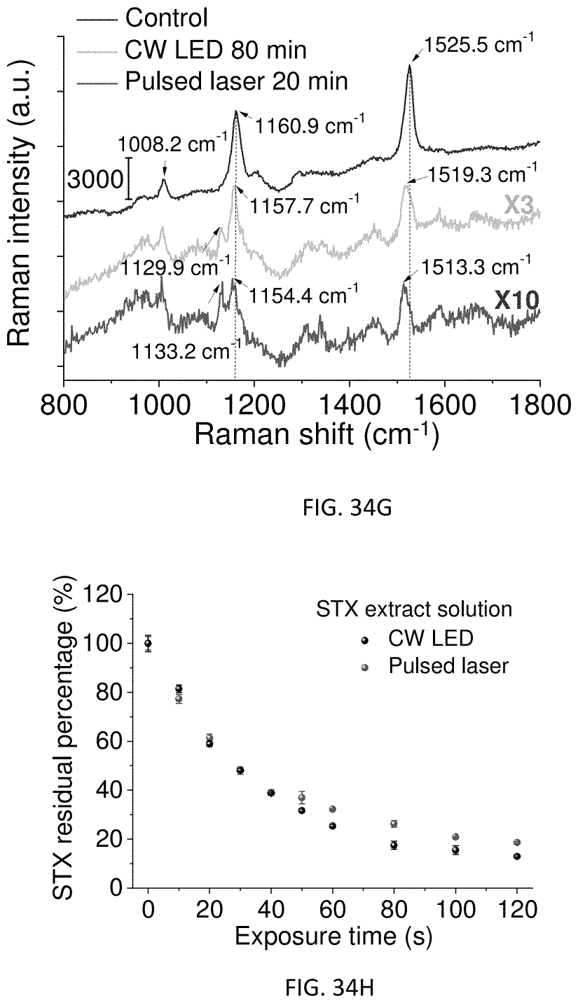

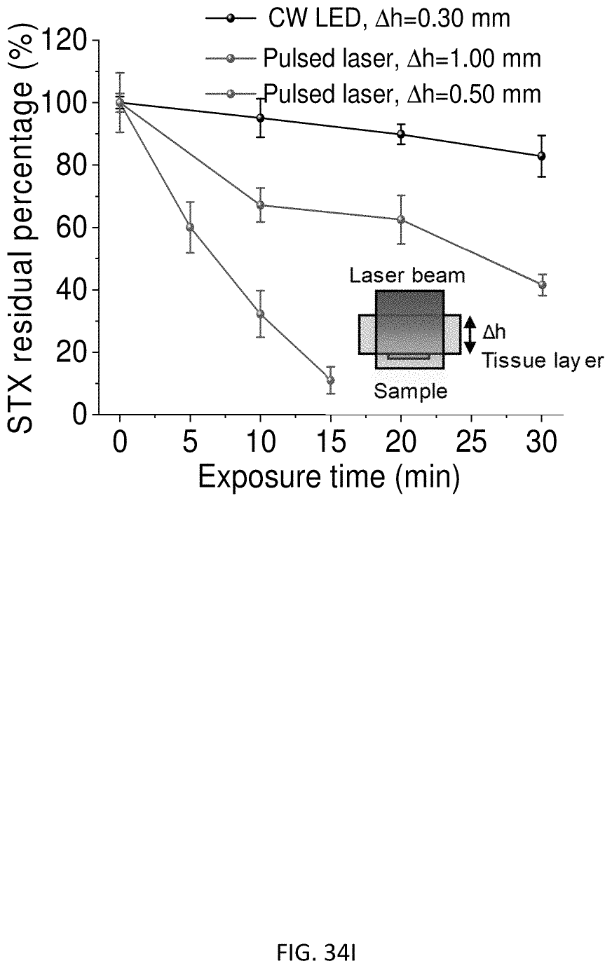

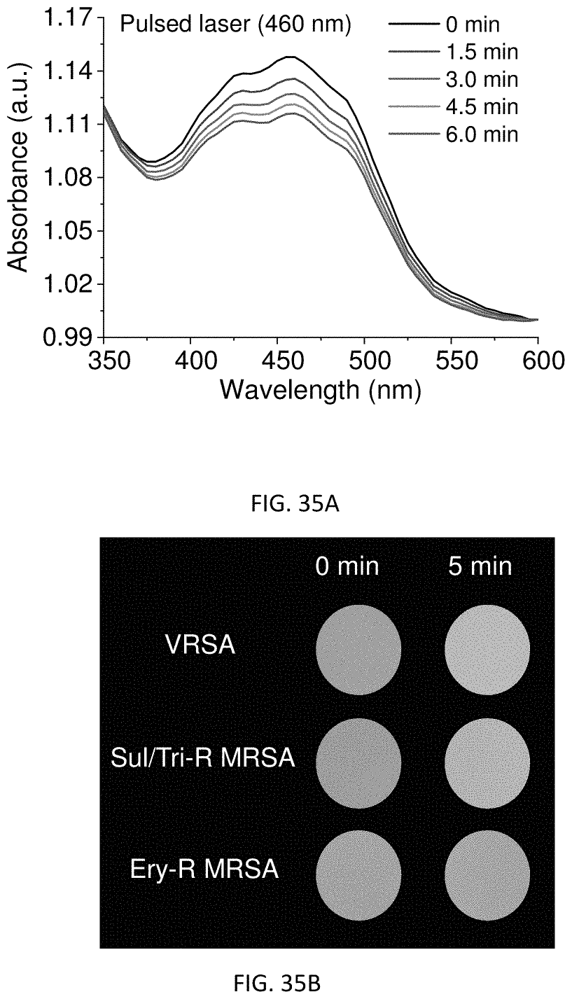

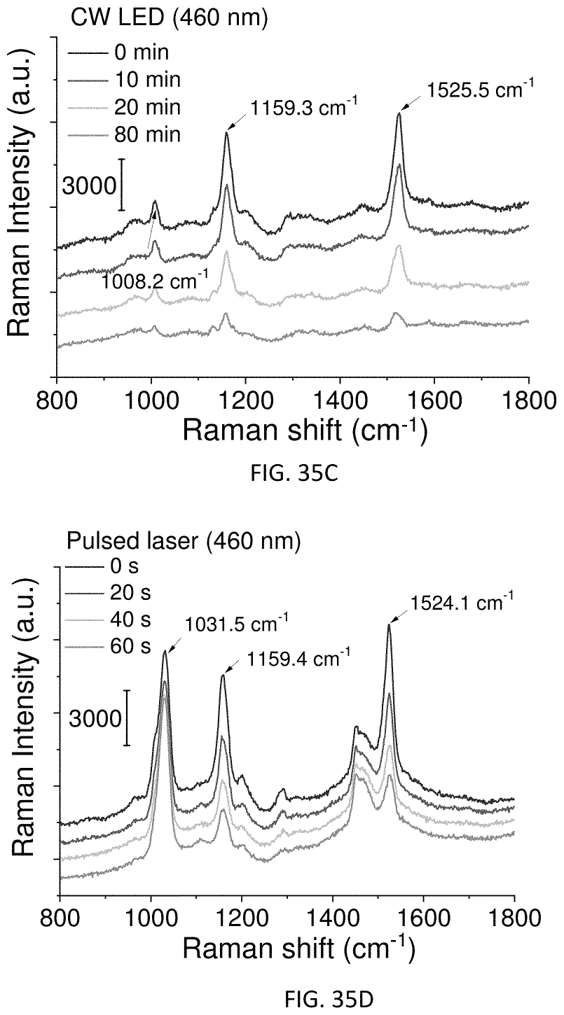

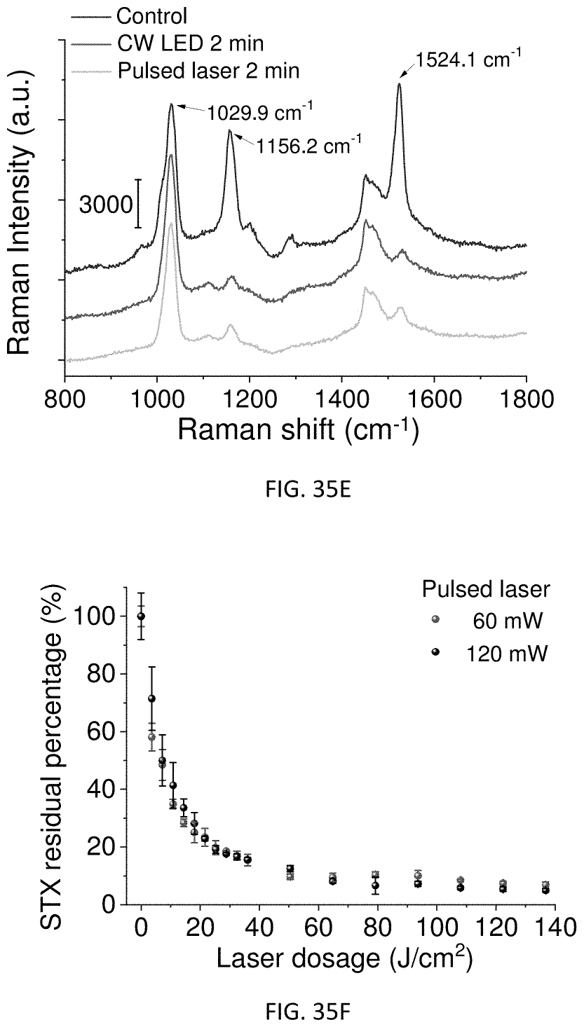

In order to test the hypothesis that STX is the molecular target of photons in the entire blue range, we directly exposed high-concentration stationary-phase MRSA colony to a wavelength-tunable laser beam in a wide-field illumination configuration as shown in FIG. 34a. Strikingly, the distinctive golden color of MRSA colony fades quickly over time when the wavelengths were tuned into the blue (400-490 nm) wavelength range (e.g. the images of MRSA colony with 460 nm illumination wavelength in FIG. 34a). As the golden colony color is originated from STX pigment, such color-fading phenomenon suggests that STX is subject to photolysis (molecular structure of STX shown in FIG. 34a). In order to further validate this point, we applied resonance Raman spectroscopy to quantify STX content in MRSA cells by taking advantage of its high sensitivity, molecular specificity, and linear concentration dependence¹⁶. STX in MRSA shows three characteristic Raman peaks around 1008 (methyl rocking), 1161 (C—C stretch), and 1525 cm⁻¹ (C═C stretch), respectively, corresponding to their specific molecular vibrational modes (FIG. 34b). With increased laser treatment time, we observed dramatically decreased peak amplitude for all three Raman bands, suggesting the cleavage of both C—C and C═C bonds that constitutes the polyene chain of STX (FIG. 34b). As a result, the unsaturated tail of STX, the nine conjugated C═C double bonds, is decomposed or truncated, as confirmed by mass spectrometry¹⁷. In contrast, when we blocked STX biosynthesis in S. aureus by knocking down CrtM, namely S. aureus ΔCrtM, its colony turns colorless and shows no detectable peaks for all three Raman bands, confirming that these Raman bands are exclusively from STX (FIG. 34c). With fixed laser power and dosage (50 mW, 5 min exposure time), MRSA colonies were further illuminated at different laser wavelengths and STX photolysis efficiency calculated using the Raman peak amplitude at 1161 cm⁻¹ before and after illumination. The results in FIG. 34d indicate that STX is subject to effective photolysis in the entire blue wavelength range (400-490 nm) with significantly reduced efficiency when above 500 nm. This efficiency curve matches the absorption spectrum of STX as photolysis is grounded on the absorption of chromophores (FIG. 35a). The effective STX photolysis induces significant absorption change, which is directly reflected on the absorption spectra of MRSA bacterial solution (FIG. 35a). By compromising the STX photolysis efficiency and optical penetration, 460-480 nm is the preferable optical window (460 nm illumination wavelength was applied in the following studies). Notably, STX photolysis behavior is not only limited to MRSA, but broadly shown on vancomycin-resistant S. aureus (VRSA) and other clinically isolated multi-drug resistant S. aureus strains (FIG. 35b and FIG. 35e; their minimum inhibitory concentrations shown in Table 2), as more than 90% of all S. aureus human clinical isolates generate this golden pigment¹⁸. Collectively, these results suggest that STX is the molecular target of photons or lasers in the entire blue range.

TABLE 1
Statistical results of fold change of 200 of cytokines from four
different groups. SEM means standard error of mean.

| | Groups | | | | | | | |
|---|---|---|---|---|---|---|---|---|
| | | | | | | | Blue | Blue |
| | Blue | Blue | | | Fusidic | Fusidic | light + | light + |
| | light- | light- | H₂O₂- | H₂O₂- | acid- | acid- | H₂O₂- | H₂O₂- |
| | treated | treated | treated | treated | treated | treated | treated | treated |
| Cytokines | (mean) | (SEM) | (mean) | SEM | (mean) | (SEM) | (mean) | (SEM) |
| AR | 0.0253 | 0.0763 | −0.1343 | 0.0330 | 0.6274 | 0.1087 | −0.1069 | 0.0407 |
| Axl | −0.3665 | 0.0116 | −0.1455 | 0.0215 | 0.0523 | 0.0026 | −0.4544 | 0.0135 |
| CD27L | 0.2131 | 0.0643 | 0.1373 | 0.0459 | −0.0491 | 0.0925 | −0.0479 | 0.0727 |
| CD30 | −0.2171 | 0.0572 | −0.0510 | 0.0239 | 0.0137 | 0.0504 | −0.2314 | 0.0749 |
| CD40 | −0.3040 | 0.0331 | −0.1007 | 0.0294 | 0.3337 | 0.0743 | −0.3057 | 0.0519 |
| CXCL16 | 0.2615 | 0.0414 | −0.2162 | 0.0351 | 0.2447 | 0.0692 | −0.1737 | 0.0265 |
| EGF | 0.2468 | 0.0459 | −0.2293 | 0.0651 | 0.0517 | 0.0729 | −0.2807 | 0.0398 |
| E-selectin | −0.1757 | 0.0250 | −0.1083 | 0.0177 | −0.2004 | 0.0209 | −0.4839 | 0.0133 |
| Fractalkine | 0.4714 | 0.1334 | 0.4416 | 0.1803 | −0.2364 | 0.3307 | −0.3121 | 0.2979 |
| GITR | 0.0287 | 0.0995 | −0.1455 | 0.0721 | 0.3966 | 0.0650 | 0.0465 | 0.0672 |
| HGF | 0.3286 | 0.0154 | 0.2749 | 0.0216 | 0.2701 | 0.0221 | 0.2357 | 0.0306 |
| IGFBP-2 | −0.0760 | 0.0183 | 0.0153 | 0.0161 | −0.0879 | 0.0174 | −0.1529 | 0.0262 |
| IGFBP-3 | −0.2336 | 0.0106 | −0.2116 | 0.0150 | −0.2120 | 0.0029 | −0.4210 | 0.0134 |
| IGFBP-5 | −0.1284 | 0.0098 | 0.0442 | 0.0247 | −0.0782 | 0.0200 | −0.1908 | 0.0205 |
| IGFBP-6 | −0.1894 | 0.0325 | −0.2308 | 0.0264 | −0.2897 | 0.0141 | −0.5027 | 0.0282 |
| IGF-1 | 0.0822 | 0.0085 | 0.2487 | 0.0099 | −0.4445 | 0.0034 | −0.3178 | 0.0025 |
| IL-12p70 | 0.0238 | 0.0095 | −0.1362 | 0.0201 | 0.0029 | 0.0236 | −0.0909 | 0.0295 |
| IL-17E | −0.2118 | 0.0335 | −0.3214 | 0.0244 | −0.1800 | 0.0079 | −0.3887 | 0.0604 |
| IL-17F | −0.0032 | 0.0262 | 0.6993 | 0.0530 | 0.2592 | 0.0461 | 0.0647 | 0.0328 |
| IL-1ra | −0.0233 | 0.0048 | 0.1670 | 0.0132 | 0.3851 | 0.0233 | −0.1671 | 0.0095 |
| IL-2 Ra | −0.0725 | 0.0265 | 0.2015 | 0.0391 | 0.1879 | 0.0242 | −0.0608 | 0.0375 |
| IL-20 | −0.1622 | 0.0467 | −0.1274 | 0.0482 | 0.0953 | 0.0715 | −0.1997 | 0.0352 |
| IL-23 | 0.2510 | 0.1121 | 0.1392 | 0.1553 | 0.2627 | 0.1342 | 0.6352 | 0.2227 |
| IL-28 | 0.1631 | 0.0296 | 0.2046 | 0.0485 | −0.1588 | 0.0353 | −0.1617 | 0.0335 |
| I-TAC | −0.0940 | 0.0819 | −0.1457 | 0.1209 | 0.3744 | 0.1874 | −0.0088 | 0.1025 |
| MDC | 0.2800 | 0.0209 | 0.5488 | 0.0762 | −0.0764 | 0.0156 | 0.5404 | 0.0882 |
| MIP-2 | 0.0488 | 0.0222 | −0.0277 | 0.0076 | −0.0070 | 0.0018 | −0.2246 | 0.1166 |
| MIP-3a | −0.1213 | 0.0728 | 0.3267 | 0.1251 | 0.3460 | 0.1535 | −0.0532 | 0.0893 |
| OPN | −0.3248 | 0.0228 | −0.2307 | 0.0105 | 0.8216 | 0.0675 | −0.3776 | 0.0184 |
| OPG | −0.0834 | 0.0274 | −0.2142 | 0.0205 | 0.9914 | 0.0447 | −0.1911 | 0.0649 |
| Prolactin | 0.0326 | 0.0264 | 0.2852 | 0.2180 | 0.4580 | 0.2106 | 0.0106 | 0.1234 |
| Pro-MMP-9 | 0.0533 | 0.0035 | 0.0213 | 0.0104 | 0.0197 | 0.0078 | 0.0371 | 0.0119 |
| P-selectin | −0.3221 | 0.0083 | −0.4006 | 0.0040 | −0.2374 | 0.0017 | −0.6809 | 0.0037 |
| Resistin | −0.0653 | 0.0043 | −0.3623 | 0.0086 | 0.2752 | 0.0093 | −0.4256 | 0.0110 |
| SCF | −0.1139 | 0.0489 | 0.1915 | 0.1865 | 0.1498 | 0.1371 | 0.0971 | 0.0282 |
| SDF-1a | −0.0106 | 0.0562 | −0.0807 | 0.0239 | 0.1707 | 0.0490 | 0.0789 | 0.0302 |
| TPO | 0.0443 | 0.0135 | −0.0215 | 0.0873 | 0.2663 | 0.0575 | −0.2020 | 0.0404 |
| VCAM-1 | −0.2367 | 0.0029 | −0.1357 | 0.0117 | −0.0283 | 0.0066 | −0.4501 | 0.0076 |
| VEGF | 0.0143 | 0.0232 | −0.0295 | 0.0258 | 0.0403 | 0.0167 | −0.2100 | 0.0319 |
| VEGF-D | 0.0806 | 0.0440 | −0.0146 | 0.0526 | 0.0367 | 0.0055 | −0.3018 | 0.0227 |
| bFGF | 0.2019 | 0.0951 | −0.2542 | 0.0822 | −0.4981 | 0.0511 | −0.6184 | 0.0076 |
| BLC | −0.5350 | 0.0778 | −0.1960 | 0.0503 | −0.3200 | 0.2049 | −0.4361 | 0.1007 |
| CD30L | −0.0464 | 0.0516 | −0.0733 | 0.0442 | −0.0682 | 0.0819 | −0.1525 | 0.0262 |
| Eotaxin | 0.7177 | 0.3852 | 0.8584 | 0.3504 | 0.8019 | 0.6864 | 0.9933 | 0.7221 |
| Eotaxin-2 | −0.0452 | 0.1538 | −0.5961 | 0.2699 | 0.0292 | 0.0267 | −0.0520 | 0.2651 |
| Fas L | 0.2062 | 0.2190 | −0.2203 | 0.0392 | −0.4112 | 0.1607 | −0.6607 | 0.0141 |
| G-CSF | 0.0729 | 0.0174 | −0.4083 | 0.0165 | 0.1394 | 0.0159 | −0.6434 | 0.0095 |
| GM-CSF | −0.2760 | 0.0372 | −0.1960 | 0.0233 | −0.1708 | 0.0467 | 0.1265 | 0.0466 |
| ICAM-1 | 1.1571 | 0.3613 | −0.0362 | 0.0593 | 1.5512 | 0.0763 | −0.4232 | 0.0112 |
| IFNg | −0.0471 | 0.0315 | −0.2366 | 0.0325 | −0.0599 | 0.0456 | −0.2612 | 0.0433 |
| IL-1a | 0.0649 | 0.0200 | 0.0141 | 0.0258 | −0.0503 | 0.0151 | −0.1406 | 0.0271 |
| IL-1b | −0.1198 | 0.0797 | 0.2075 | 0.0822 | −0.2158 | 0.0442 | 0.2268 | 0.0879 |
| IL-2 | −0.0011 | 0.0608 | −0.1860 | 0.0312 | 0.0606 | 0.0464 | −0.2761 | 0.0298 |
| IL-3 | −0.0171 | 0.0455 | −0.1781 | 0.0431 | −0.1398 | 0.0616 | −0.1911 | 0.0328 |
| IL-4 | −0.1276 | 0.0360 | −0.2011 | 0.0249 | −0.0096 | 0.0534 | −0.3169 | 0.0233 |
| IL-5 | −0.0918 | 0.0720 | −0.3106 | 0.0187 | −0.0953 | 0.0558 | −0.2998 | 0.0513 |
| IL-6 | 0.0085 | 0.0663 | −0.2775 | 0.0467 | 0.2682 | 0.0345 | −0.2068 | 0.0245 |
| IL-7 | −0.1130 | 0.2833 | −0.0083 | 0.4243 | 0.1116 | 0.1899 | −0.1273 | 0.1945 |
| IL-10 | −0.1209 | 0.0256 | −0.1759 | 0.0236 | −0.0351 | 0.0477 | −0.1589 | 0.0408 |
| IL-12p40 | 0.1462 | 0.0502 | 0.0200 | 0.0622 | 0.0324 | 0.0810 | −0.1654 | 0.0628 |
| IL-13 | −0.3103 | 0.1384 | −0.4855 | 0.1559 | −0.5654 | 0.2744 | 0.0537 | 0.2934 |
| IL-15 | 0.1103 | 0.1950 | −0.1351 | 0.0599 | −0.0506 | 0.0964 | 0.1134 | 0.1189 |
| IL-17 | −0.1805 | 0.0238 | 1.4006 | 0.1593 | 0.2684 | 0.0368 | −0.2598 | 0.0373 |
| IL-21 | 0.2484 | 0.3366 | −0.5851 | 0.2910 | −0.4052 | 0.2764 | −0.1496 | 0.2755 |
| KC | 0.0887 | 0.0175 | −0.0602 | 0.0226 | 0.0909 | 0.0184 | −0.2108 | 0.0195 |
| Leptin | 0.4501 | 0.0195 | −0.1401 | 0.2313 | −0.1199 | 0.0094 | −0.5289 | 0.0781 |
| LIX | 0.2186 | 0.0849 | −0.2304 | 0.0473 | −0.0313 | 0.0181 | −0.4204 | 0.0192 |
| MCP-1 | 0.2899 | 0.0827 | 0.9431 | 0.0604 | 0.0071 | 0.0722 | 0.3119 | 0.0795 |
| MCP-5 | 0.4730 | 0.1836 | −0.8808 | 0.0331 | −0.0915 | 0.0493 | −0.4174 | 0.2217 |
| MCSF | 0.2029 | 0.0613 | −0.0002 | 0.0172 | −0.5503 | 0.0202 | −0.3853 | 0.0330 |
| MIG | 0.6206 | 0.1996 | −0.1127 | 0.0826 | 0.5378 | 0.1103 | 0.3628 | 0.0517 |
| MIP-1a | 0.1419 | 0.0193 | −0.3107 | 0.0083 | −0.3165 | 0.0278 | −0.6672 | 0.0074 |
| MIP-1g | 0.1167 | 0.0336 | −0.0241 | 0.0197 | 0.0304 | 0.0144 | −0.2576 | 0.0105 |
| PF4 | −0.3297 | 0.1071 | −0.7429 | 0.0504 | −0.6256 | 0.0740 | −0.6165 | 0.1723 |
| RANTES | −0.1383 | 0.2254 | −0.4249 | 0.2078 | −0.2545 | 0.1164 | −0.2548 | 0.1306 |
| TARC | 0.2738 | 0.2655 | −0.4322 | 0.2026 | 0.3205 | 0.1708 | −0.7453 | 0.1122 |
| TCA-3 | −0.2486 | 0.0114 | −0.2756 | 0.0436 | −0.1264 | 0.0578 | −0.2991 | 0.0290 |
| TNF RI | −0.1672 | 0.0122 | −0.2939 | 0.0366 | −0.1306 | 0.0591 | −0.5615 | 0.0318 |
| TNF RII | −0.0452 | 0.0191 | −0.1233 | 0.0203 | −0.2811 | 0.0052 | −0.4056 | 0.0120 |
| TNFα | −0.0792 | 0.0471 | 0.1039 | 0.0407 | −0.4306 | 0.0182 | −0.1717 | 0.0477 |
| 6Ckine | −0.2017 | 0.0538 | −0.3474 | 0.0359 | 0.0930 | 0.0636 | −0.3244 | 0.0782 |
| Activin A | 0.2080 | 0.1248 | 0.0097 | 0.1383 | 0.2305 | 0.1051 | −0.1235 | 0.0752 |
| ADAMTS1 | 0.1622 | 0.0835 | 0.1068 | 0.0187 | 0.2350 | 0.0792 | 0.0183 | 0.0304 |
| Adiponen | −0.0615 | 0.0556 | −0.0278 | 0.0384 | 0.0147 | 0.0119 | −0.0459 | 0.0212 |
| ANG-3 | −0.3493 | 0.0690 | −0.1019 | 0.1171 | 0.0313 | 0.1455 | −0.1613 | 0.0772 |
| ANGPTL3 | −0.4744 | 0.2343 | −0.1222 | 0.1516 | −0.2231 | 0.1000 | −0.2169 | 0.1404 |
| Artemin | 0.0300 | 0.0845 | 0.0433 | 0.0216 | 0.4218 | 0.4931 | 1.0812 | 0.8646 |
| CCL28 | −0.1679 | 0.1123 | −0.6841 | 0.2340 | 1.0791 | 1.3719 | −0.0230 | 0.0453 |
| CD36 | −0.4046 | 0.0415 | −0.4456 | 0.0342 | 0.0674 | 0.0331 | −0.2357 | 0.0421 |
| Chordin | −0.1152 | 0.0448 | −0.0093 | 0.0715 | −0.0599 | 0.0534 | −0.1698 | 0.0453 |
| CRP | −0.1386 | 0.0621 | −0.2262 | 0.0133 | −0.0863 | 0.0390 | −0.6176 | 0.0347 |
| E-Cadherin | −0.0110 | 0.0399 | −0.1379 | 0.0244 | 0.1848 | 0.0312 | −0.0999 | 0.0321 |
| Epigen | −0.1274 | 0.0941 | −0.1588 | 0.0635 | −0.0490 | 0.1059 | −0.0790 | 0.0640 |
| Epiregulin | 0.0589 | 0.1819 | −0.2878 | 0.1812 | −0.2220 | 0.1128 | −0.3278 | 0.1062 |
| Fas | −0.1400 | 0.0892 | −0.3001 | 0.0746 | 0.0163 | 0.1182 | −0.1501 | 0.1405 |
| Galectin-7 | −0.3203 | 0.0284 | −0.3150 | 0.0754 | 0.7990 | 0.0420 | −0.2551 | 0.0257 |
| gp130 | −0.0160 | 0.0246 | 0.1048 | 0.2725 | 0.5867 | 0.1372 | −0.3457 | 0.1337 |
| Granzyme B | −0.0904 | 0.0532 | 0.2145 | 0.0847 | 0.5603 | 0.0682 | −0.1550 | 0.0897 |
| Gremlin | −0.5466 | 0.3567 | −0.8057 | 0.1047 | 4.9707 | 2.4157 | −0.6070 | 0.0440 |
| IFNg R1 | −0.2039 | 0.0343 | −0.2832 | 0.0240 | 0.0709 | 0.0445 | −0.2927 | 0.0458 |
| IL-17B | −0.1487 | 0.0974 | −0.2485 | 0.1711 | −0.0513 | 0.1850 | −0.3900 | 0.1458 |
| IL-17B R | −0.0890 | 0.0572 | 0.0438 | 0.2243 | 0.4884 | 0.0727 | 0.0476 | 0.1280 |
| IL-22 | −0.0037 | 0.0483 | −0.5330 | 0.1964 | −0.0487 | 0.0884 | 0.2242 | 0.2403 |
| MIP-1b | −0.1873 | 0.0215 | −0.4967 | 0.0257 | −0.5769 | 0.0187 | −0.8282 | 0.0084 |
| MMP-2 | −0.4581 | 0.0237 | −0.5768 | 0.0277 | 0.1026 | 0.0759 | −0.5532 | 0.0261 |
| MMP-3 | 0.0194 | 0.0341 | −0.0983 | 0.0203 | −0.1230 | 0.0304 | −0.2390 | 0.0512 |
| MMP-10 | 0.1624 | 0.0948 | −0.1476 | 0.0691 | 0.3470 | 0.1412 | −0.1524 | 0.0660 |
| PDGF-AA | −0.1688 | 0.0637 | −0.4666 | 0.0535 | 0.4578 | 0.0075 | −0.3440 | 0.1022 |
| Persephin | −0.0917 | 0.1338 | −0.3386 | 0.0476 | 0.5348 | 0.4577 | −0.3563 | 0.1026 |
| sFRP-3 | 0.1509 | 0.1414 | −0.1892 | 0.0965 | 0.0607 | 0.1143 | −0.1970 | 0.0940 |
| Shh-N | −0.1287 | 0.0334 | −0.1527 | 0.0915 | −0.0849 | 0.0589 | −0.1079 | 0.0621 |
| SLAM | −0.1210 | 0.0730 | −0.0883 | 0.0869 | 0.1871 | 0.1523 | 0.0213 | 0.2118 |
| TCK-1 | −0.2505 | 0.0355 | −0.4843 | 0.0284 | −0.0185 | 0.0460 | −0.5671 | 0.0480 |
| TECK | 0.2360 | 0.1331 | 0.1734 | 0.1358 | 0.4529 | 0.0196 | 0.1942 | 0.2169 |
| TGFb1 | −0.0744 | 0.0333 | −0.1570 | 0.0674 | 0.0743 | 0.0947 | −0.2710 | 0.0161 |
| TRANCE | −0.0561 | 0.0631 | 0.0878 | 0.1184 | 0.0310 | 0.1001 | −0.1081 | 0.0971 |
| TremL1 | −0.2420 | 0.0475 | −0.2695 | 0.0400 | −0.0440 | 0.0605 | −0.3751 | 0.0647 |
| TWEAK | −0.1893 | 0.0429 | −0.1667 | 0.0588 | −0.0621 | 0.0444 | −0.2538 | 0.0509 |
| VEGF-B | −0.0310 | 0.0315 | −0.0693 | 0.0743 | 0.0230 | 0.0936 | −0.1112 | 0.0636 |
| VEGF R2 | −0.1005 | 0.0875 | −0.1809 | 0.1361 | 0.0949 | 0.0795 | −0.1262 | 0.0834 |
| 4-1BB | −0.1530 | 0.0340 | −0.2733 | 0.0387 | −0.0924 | 0.0238 | −0.2811 | 0.0400 |
| ACE | −0.6697 | 0.0088 | −0.8627 | 0.0085 | −0.3271 | 0.0188 | −0.9222 | 0.0040 |
| ALK-1 | −0.0477 | 0.0398 | −0.1821 | 0.0440 | −0.0491 | 0.0488 | −0.1008 | 0.0807 |
| CT-1 | −0.1602 | 0.0470 | −0.3499 | 0.0647 | −0.0581 | 0.0542 | −0.4161 | 0.0616 |
| CD27 | 0.6073 | 0.5536 | 0.1670 | 0.2393 | 0.2818 | 0.4635 | −0.0552 | 0.2504 |
| CD40L | −0.0735 | 0.0299 | −0.2549 | 0.0348 | 0.0244 | 0.0566 | −0.1621 | 0.0480 |
| CTLA4 | −0.2401 | 0.0731 | −0.1747 | 0.0581 | 0.2149 | 0.0247 | −0.2247 | 0.0479 |
| Decorin | 0.0408 | 0.0208 | −0.0051 | 0.0142 | −0.0222 | 0.0116 | −0.0389 | 0.0121 |
| Dkk-1 | −0.0611 | 0.1164 | −0.4175 | 0.0776 | 0.1157 | 0.1753 | −0.3092 | 0.0689 |
| Dtk | −0.1949 | 0.1068 | −0.3288 | 0.1031 | 0.1217 | 0.1707 | −0.3991 | 0.1426 |
| Endoglin | −0.6549 | 0.0189 | −0.6886 | 0.0102 | 0.4267 | 0.0442 | −0.8240 | 0.0112 |
| Fcg RIIB | 0.1707 | 0.2691 | 0.2388 | 0.1523 | 0.0602 | 0.1175 | 0.0869 | 0.1740 |
| Flt-3L | −0.2097 | 0.0498 | −0.2615 | 0.0403 | 0.1619 | 0.0536 | −0.4176 | 0.0342 |
| Galectin-1 | −0.2113 | 0.0094 | −0.1195 | 0.0029 | 0.0284 | 0.0165 | −0.2467 | 0.0118 |
| Galectin-3 | 0.0829 | 0.0328 | 0.0550 | 0.0434 | 0.0624 | 0.0393 | −0.0718 | 0.0049 |
| Gas 1 | −0.7289 | 0.0135 | −0.7018 | 0.0130 | 0.0715 | 0.0443 | −0.8007 | 0.0075 |
| Gas 6 | −0.3223 | 0.0735 | −0.3085 | 0.0842 | −0.0661 | 0.1169 | −0.4867 | 0.0492 |
| GITR L | −0.3959 | 0.0856 | −0.0425 | 0.0204 | 0.2327 | 0.2062 | 0.2469 | 0.2119 |
| HAI-1 | 0.1479 | 0.0643 | −0.0794 | 0.0321 | 0.2271 | 0.1224 | −0.0636 | 0.0616 |
| HGF R | 0.0369 | 0.0107 | 0.0240 | 0.0153 | 0.0292 | 0.0500 | −0.1031 | 0.0468 |
| IL-1 R4 | −0.2815 | 0.1231 | 0.0090 | 0.0505 | −0.0208 | 0.1191 | −0.3740 | 0.0468 |
| IL-3 Rb | 0.0853 | 0.0438 | −0.0953 | 0.0636 | 0.0789 | 0.0915 | −0.1247 | 0.0511 |
| IL-9 | 0.1911 | 0.0691 | 0.0482 | 0.0508 | 0.1408 | 0.0456 | −0.0427 | 0.0813 |
| JAM-A | −0.4151 | 0.0115 | −0.2998 | 0.0142 | 0.3062 | 0.0168 | −0.3706 | 0.0141 |
| Leptin R | 0.1395 | 0.0794 | −0.1563 | 0.0680 | 0.1134 | 0.0633 | −0.0508 | 0.0493 |
| L-Selectin | −0.0584 | 0.0108 | −0.1755 | 0.0156 | −0.3559 | 0.0035 | −0.4079 | 0.0735 |
| Lymphotactin | 0.1433 | 0.0601 | 0.0191 | 0.0048 | 0.0908 | 0.0649 | −0.0217 | −0.0640 |
| MadCAM-1 | −0.0329 | 0.0944 | −0.3032 | −0.1074 | −0.0787 | 0.0310 | −0.3479 | −0.0915 |
| MFG-E8 | −0.1803 | 0.0985 | −0.1414 | 0.1428 | 0.4647 | 0.1403 | −0.2667 | 0.1627 |
| MIP-3b | −0.2876 | 0.0513 | −0.1993 | 0.0255 | 0.0690 | 0.0673 | −0.2626 | 0.0466 |
| Neprilysin | −0.1241 | 0.0550 | −0.2555 | 0.0249 | 0.5936 | 0.0602 | −0.1471 | 0.0492 |
| Pentraxin 3 | −0.7139 | 0.0144 | −0.7179 | 0.0093 | −0.6735 | 0.0176 | −0.7512 | 0.0104 |
| RAGE | 0.8636 | 0.2700 | −0.0350 | 0.1155 | 0.1727 | 0.2876 | 0.4286 | 0.2164 |
| TACI | 0.5432 | 0.2929 | −0.2636 | 0.0562 | 0.4640 | 0.1721 | −0.3018 | 0.0360 |
| TREM-1 | 0.0515 | 0.0298 | 0.0703 | 0.0184 | 0.0981 | 0.0315 | 0.0117 | 0.0077 |
| TROY | −0.1253 | 0.0410 | −0.0721 | 0.0387 | 0.0517 | 0.0974 | −0.1897 | 0.0562 |
| TSLP | 0.0850 | 0.0151 | −0.1726 | 0.0151 | −0.0086 | 0.0330 | −0.1796 | 0.0443 |
| TWEAK R | −0.4665 | 0.0161 | −0.4445 | 0.0379 | −0.2341 | 0.0367 | −0.5804 | 0.0194 |
| VEGF R1 | −0.1990 | 0.0149 | −0.4184 | 0.0662 | 0.1580 | 0.0664 | −0.4483 | 0.0338 |
| VEGF R3 | 0.0627 | 0.0449 | −0.0705 | 0.0214 | 0.1061 | 0.0391 | −0.1200 | 0.0387 |
| B7-1 | 0.0247 | 0.0148 | 0.2808 | 0.0832 | −0.0561 | 0.0582 | −0.4049 | 0.0401 |
| BAFF R | 0.0229 | 0.1585 | 0.0988 | 0.0509 | 0.0687 | 0.1658 | 0.2564 | 0.2350 |
| BTC | 2.0760 | 0.4898 | 0.9018 | 0.0595 | 3.8576 | 0.5841 | 0.4695 | 0.2331 |
| C5a | 0.0615 | 0.0256 | 0.1618 | 0.0348 | 0.1215 | 0.0296 | −0.0793 | 0.0290 |
| CCL6 | −0.2209 | 0.0121 | −0.3559 | 0.0061 | −0.4027 | 0.0195 | −0.7546 | 0.0079 |
| CD48 | −0.5379 | 0.0303 | −0.3656 | 0.0460 | −0.0498 | 0.0649 | −0.4659 | 0.0151 |
| CD6 | −0.4826 | 0.0247 | −0.2795 | 0.1299 | 0.3476 | 0.3088 | −0.4601 | 0.0168 |
| Chemerin | 0.0599 | 0.0631 | −0.2510 | 0.1370 | 0.1948 | 0.2769 | −0.3033 | 0.2422 |
| Clusterin | −0.0664 | 0.0213 | 0.1123 | 0.0413 | 0.2455 | 0.0152 | −0.0353 | 0.0350 |
| Lungkine | −0.0667 | 0.0931 | −0.0954 | 0.0945 | −0.0819 | 0.0373 | −0.0777 | 0.0789 |
| Cystatin C | −0.0737 | 0.0245 | 0.0313 | 0.0278 | −0.0271 | 0.0133 | 0.0283 | 0.0171 |
| DAN | | | | | | | | |
| DLL4 | −0.1527 | 0.0099 | −0.0364 | 0.0433 | −0.0252 | 0.0551 | −0.1180 | 0.0203 |
| EDAR | 0.0980 | 0.0929 | 0.0841 | 0.0730 | −0.4592 | 0.2757 | 0.0023 | 0.0758 |
| Endocan | −0.3661 | 0.0901 | −0.0335 | 0.0915 | 0.0559 | 0.2286 | −0.5513 | 0.0921 |
| Fetuin A | −0.7771 | 0.0143 | −0.5133 | 0.0105 | −0.6614 | 0.0123 | −0.5076 | 0.0397 |
| H60 | 0.3941 | 0.2064 | −0.0250 | 0.2894 | 0.2171 | 0.1074 | −0.6484 | 0.2325 |
| IL-33 | −0.6506 | 0.0773 | −0.6370 | 0.0229 | 0.3416 | 0.0791 | −0.6861 | 0.0293 |
| IL-7 Ra | −0.3610 | 0.2570 | −0.4620 | 0.2010 | −0.7276 | 0.2177 | −0.8567 | 0.1241 |
| Kremen-1 | 0.0957 | 0.0261 | −0.4532 | 0.2739 | 0.2672 | 0.2121 | 0.0865 | 0.0424 |
| Limitin | 0.6721 | 0.3043 | 0.5441 | 0.2060 | −0.1844 | 0.3041 | 0.0561 | 0.0536 |
| Lipocalin-2 | −0.1402 | 0.0213 | −0.0669 | 0.0120 | −0.0695 | 0.0215 | −0.2269 | 0.1060 |
| LOX-1 | 0.0330 | 0.0189 | 0.0229 | 0.0122 | 0.0568 | 0.0316 | 0.0374 | 0.0218 |
| Marapsin | −0.3802 | 0.1087 | −0.0476 | 0.0430 | −0.1703 | 0.1764 | −0.4160 | 0.1584 |
| MBL-2 | −0.1568 | 0.0559 | −0.0587 | 0.1048 | 0.0893 | 0.0575 | −0.0253 | 0.1203 |
| Meteorin | 0.2441 | 0.2360 | 0.4850 | 0.1991 | 0.3361 | 0.2098 | 0.2874 | 0.1863 |
| Nope | −0.3388 | 0.0519 | −0.2914 | 0.0262 | 0.0534 | 0.0190 | −0.4400 | 0.0125 |
| NOV | −0.3379 | 0.2091 | −0.1756 | 0.0918 | −0.2053 | 0.1403 | −0.9702 | 0.0258 |
| Osteoactivin | −0.2787 | 0.1389 | 0.4709 | 0.2336 | 1.1132 | 0.3224 | 0.4250 | 0.1957 |
| OX40 Ligand | −0.2417 | 0.1367 | 0.2366 | 0.2672 | −0.1454 | 0.3266 | −0.3974 | 0.0926 |
| P-Cadherin | −0.4885 | 0.0090 | −0.0660 | 0.0326 | 0.5883 | 0.0546 | −0.1158 | 0.0448 |
| Periostin | −0.5653 | 0.0135 | −0.5519 | 0.0280 | 0.1651 | 0.0640 | −0.5594 | 0.0138 |
| PlGF-2 | −0.2314 | 0.0226 | −0.0738 | 0.0353 | −0.0798 | 0.0662 | −0.2486 | 0.0226 |
| Progranulin | −0.5637 | 0.0105 | −0.6229 | 0.0115 | −0.4424 | 0.0276 | −0.7865 | 0.0108 |
| Prostasin | −0.0635 | 0.0795 | 1.1226 | 0.2206 | 0.1030 | 0.0963 | −0.2391 | 0.2142 |
| Renin 1 | −0.5961 | 0.0248 | −0.3457 | 0.0444 | −0.4138 | 0.0163 | −0.6316 | 0.0220 |
| Testican 3 | −0.5810 | 0.2198 | −0.1883 | 0.1057 | −0.4097 | 0.0586 | −0.6049 | 0.1959 |
| TIM-1 | −0.1186 | 0.0586 | −0.1106 | 0.0663 | −0.0806 | 0.0293 | −0.1534 | 0.0832 |
| TRAIL | | | | | | | | |
| Tryptase ε | 0.0853 | 0.1731 | 0.3620 | 0.3640 | −0.0627 | 0.1395 | −0.2941 | 0.2140 |

TABLE 2
Minimum inhibitory concentrations of selected antibiotics
against the tested bacterial strains. N = 3 for each measurement.

| Clinical isolates | Antibiotics | MIC (μg/ml) | Source/description |
|---|---|---|---|
| VRSA NR-46419 | Vancomycin | 256 | Isolated in 2007 in Michigan, USA from a left plantar |
| (VRSA 9) | | | foot wound of a 54-year-old female, who recently |
| | | | received a 4-week course of vancomycin and |
| | | | levofloxacin to treat osteomyelitis of the left |
| | | | metatarsals. |
| MRSA NRS384 | Erythromycin | 64 | Isolated from a wound in Mississippi, USA. It is a |
| (MRSA USA 300) | | | community-acquired MRSA strain. |
| MRSA NRS385 | Sulfamethoxazole/ | 256 | Isolated from a bloodstream sample in Connecticut, |
| (MRSA USA 500) | trimethoprim | | USA. It is a hospital-acquired MRSA strain. |

| Bacterial strain | Antibiotics | MIC (μg/ml) |
|---|---|---|
| MRSA USA 300 | Daptomycin | 8 |
| | Gentamicin | 8 |
| | Oxacillin | 8 |
| | Ofloxacin | 0.5 |

Considering the significance of STX virulence in a MRSA-caused disease, an optimal light source that enables efficient, fast, complete, and deep depletion of STX is of great importance. Our previous study via transient absorption microscopy suggests that STX photolysis under tightly focused laser primarily follows a second-order photolysis model due to triplet-triplet annihilation: T*+T*→R+S, where R and S represent reduced and semi-oxidized forms¹⁷. The triplet excitons form with high yield via singlet fission when carotenoids self-assemble into multimer or aggregates on cell membrane. As the triplet lifetime of STX is on a microsecond scale and STX laterally assembles within FMM, a high-fluence nanosecond pulsed laser can be used to effectively populate STX molecules to their triplet state within single pulse excitation thus accelerating STX photolysis nonlinearly.

To test this hypothesis, we firstly exposed stationary-phase MRSA colony to the nanosecond pulsed laser and a continuous-wave light-emitting diode (LED) with output power of 120 mW, with wavelength centered around 460 nm, then monitored their residual STX through resonance Raman spectroscopy over different exposure time. Remarkably, the nanosecond pulsed laser shows unmatched efficiency, speed, and completeness for STX photolysis when compared with the LED, as it depletes 80% of STX in MRSA cells within less than 2 mins, whereas it takes LED more than 20 mins to reach the same efficiency (FIG. 34b,f and FIG. 35c); the STX photolysis by LED is not complete even over 80 mins illumination. The efficiency and speed come from the nonlinearity of STX annihilation enabled by nanosecond pulsed laser, consistent with the second-order fitting¹⁷ result of the decay curve (FIG. 34f). By closely examining the Raman spectra, nanosecond pulsed laser further induces significant blue shifts of these peaks; the shifts are as large as 12 and 6 cm⁻¹ for peaks at 1525 and 1161 cm⁻¹, respectively (FIG. 34g). These blue shifts provide additional evidence to support the photochemistry process in STX. In contrast, when we monitored the photolysis kinetics on STX solution extracted from MRSA pellets, nanosecond pulsed laser and LED no longer show distinctive decay curves (FIG. 34h and FIG. 35d,e). Therefore, STX photolysis speed as suggested has high concentration dependence; highly aggregated STX nonlinearly increases STX photolysis efficiency and speed. When laser pulse fluence was doubled meanwhile keeping illumination dosage the same, photolysis delay curves for nanosecond pulsed laser only show minor difference, as likely this 2-time difference in pulse fluence is minor when compared with 10⁷-time difference between nanosecond pulsed laser and continuous-wave LED under the same power (FIG. 35f). Thus, further shortened illumination time can be achieved by simply increasing pulse fluence until reaching saturation. More significantly, the high-fluence nanosecond pulsed laser enables ˜4-fold larger treatment depth when compared with LED, as more than 50% STX molecules are depleted by nanosecond pulsed laser when MRSA colonies are placed beneath a tissue layer with thickness beyond 1 mm within one cell cycle (30 mins), whereas LED barely penetrates through 300 μm tissue to reach the same efficiency (FIG. 34i, experimental schematic shown in the inset). Such effective STX photolysis in deep tissue comes from the conjugation of the photolysis nonlinearity and high photon fluence of pulsed laser, as the photons fluence of pulsed laser is several orders of magnitude higher than that of low-level light sources (e.g. LED) even through a thick tissue layer. The extended depth is sufficient to penetrate and treat MRSA biofilms (thickness typically ranging from a few micrometers to several hundreds of micrometers²¹), which are normally difficult to treat by antibiotics due to biofilm-mediated inacitvation. Notably, the power and dosage for nanosecond pulsed blue laser in this study are below the American National Standards Institute (ANSI) safety limit for human skin exposure to lasers at 460 nm. In contrast to continuous-wave LED, nanosecond pulsed laser further eliminates potential photothermal issues as the temperature rise on human skin is quite small (<5 degree). Collectively, these results suggest that high-fluence short-pulsed blue laser is the superior light source to deplete STX in MRSA quickly, effectively, completely, and safely.

### Example 2. Photo-Disasembly of Functional Membrane Microdomains: Membrane

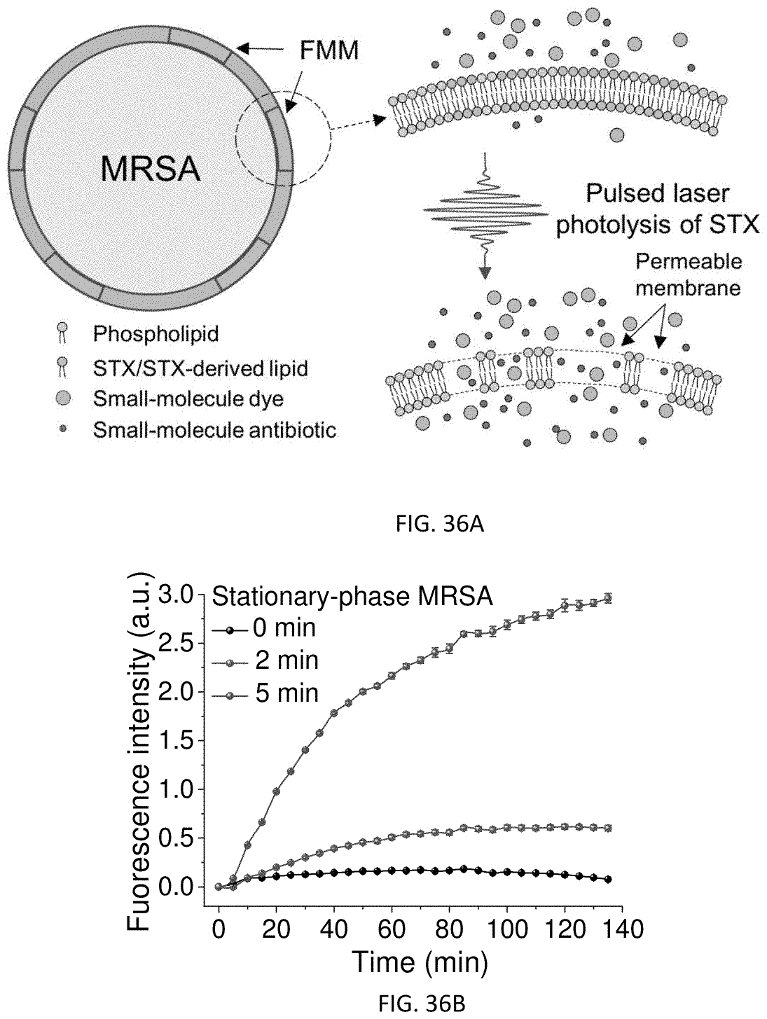

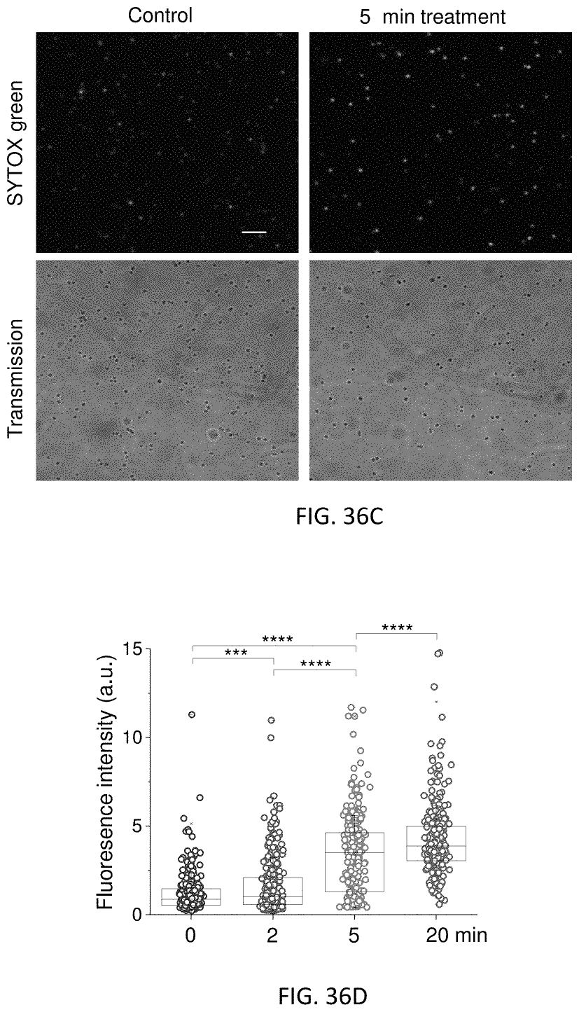

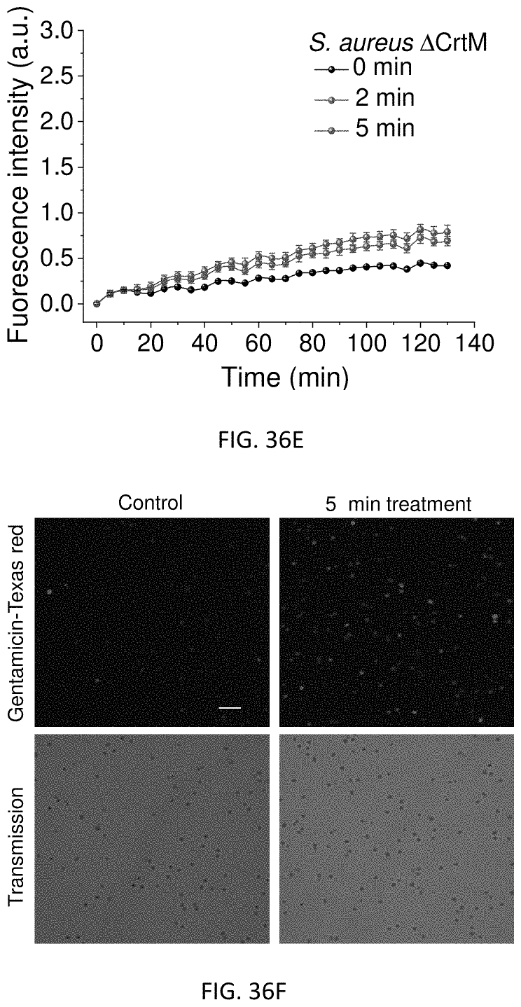

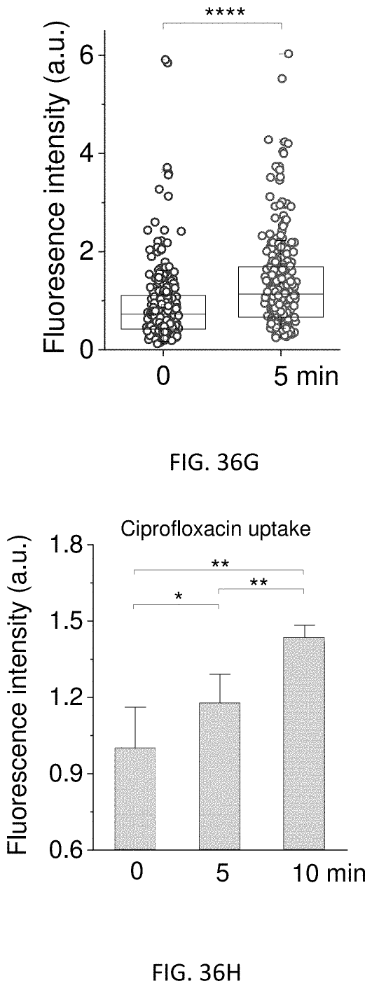

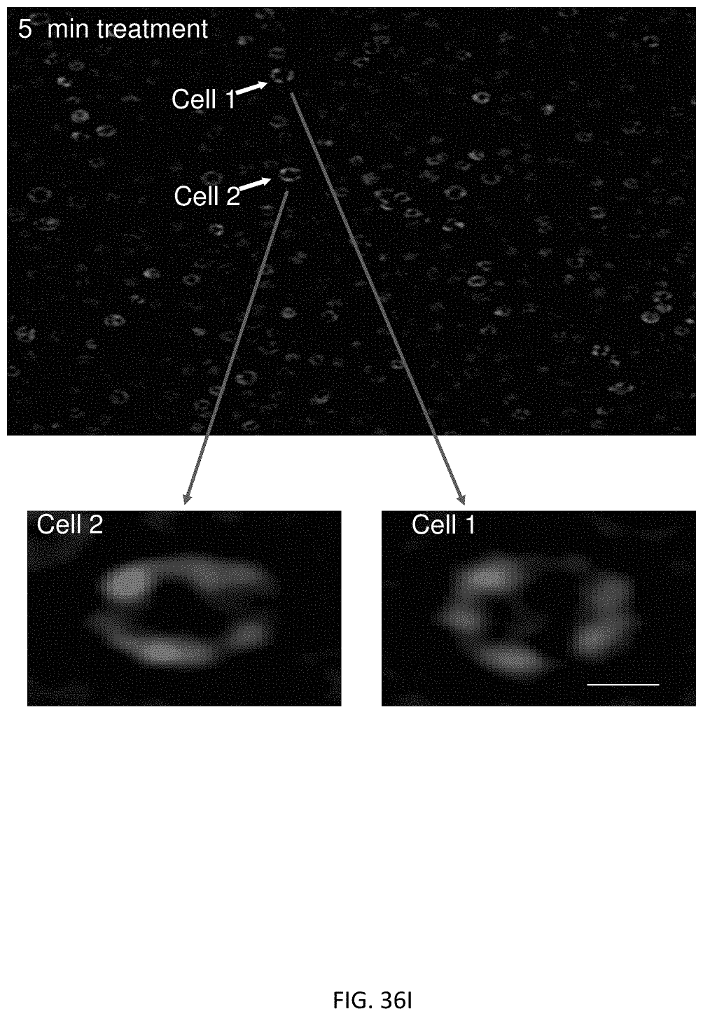

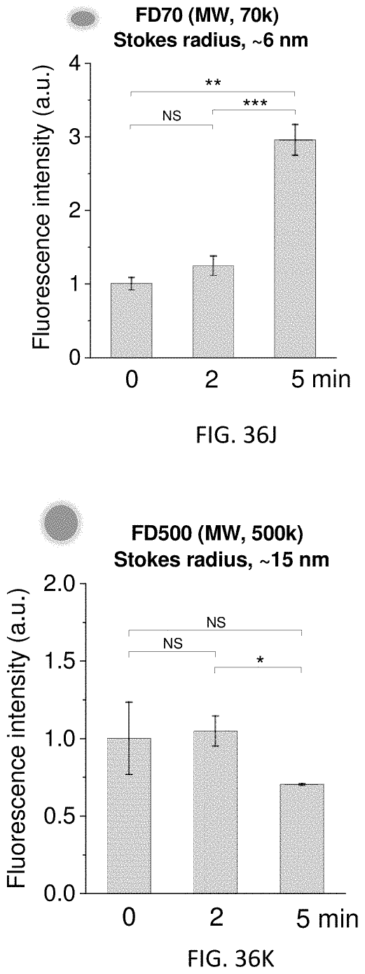

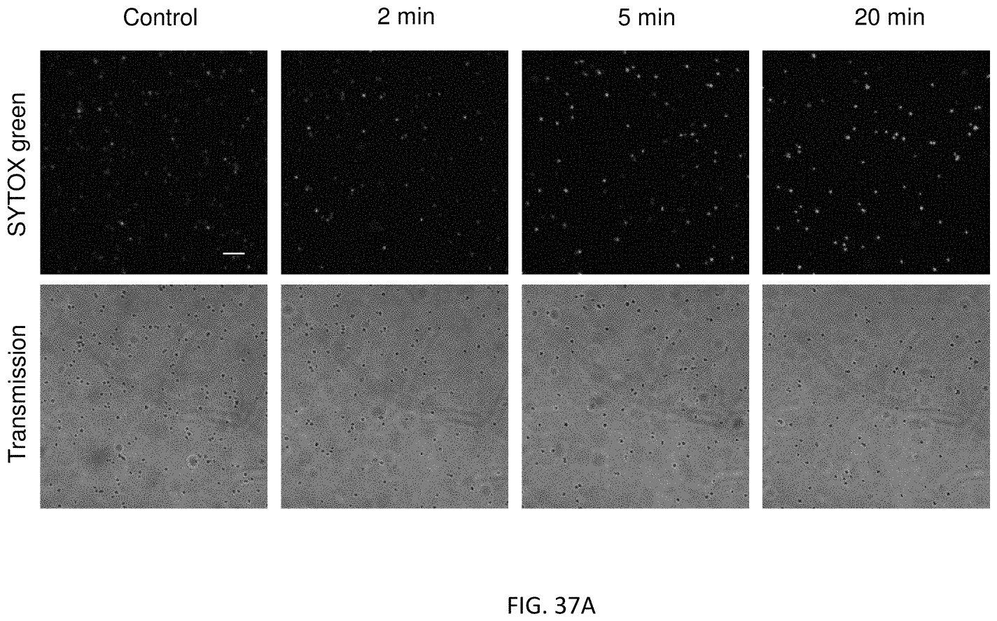

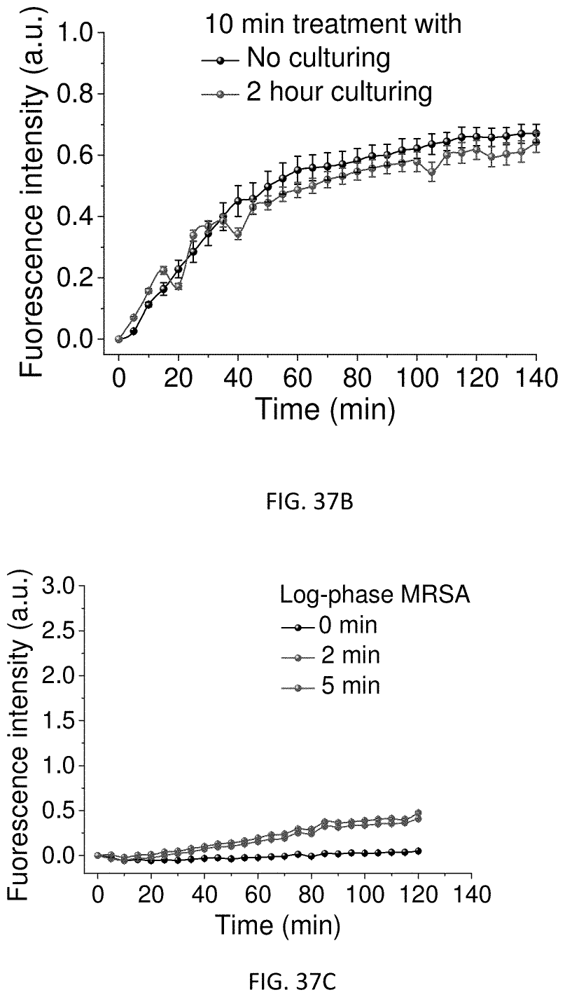

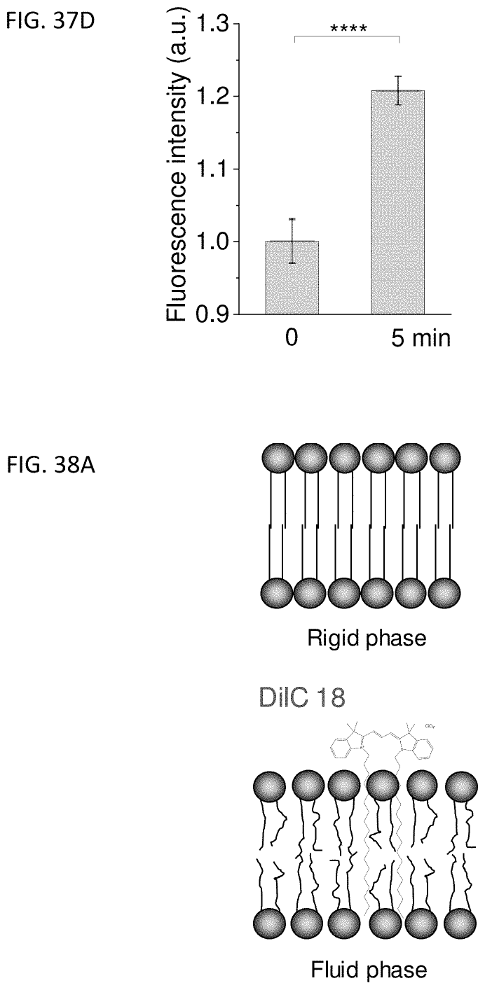

STX is known acting as the constituent lipid of FMM, which are embedded in the lipid bilayer of virulent S. aureus strains and implicated in maintenance of membrane integrity. Therefore, we hypothesize that STX photolysis disrupts membrane integrity by increasing membrane permeability, thus facilitating the intracellular accumulation of small-molecule dyes or antibiotics via passive diffusion (FIG. 36a). To prove this point, membrane permeability with or without laser treatment was evaluated in real time by SYTOX green (600 Da), a fluorescent dye for nucleic acids stain of cells only with compromised membrane. With increased laser treatment time, a significantly larger and faster uptake of SYTOX green is observed, indicating severely compromised cell membranes; whereas cells without laser treatment show negligible uptake, which validates the role of STX on membrane integrity (FIG. 36b). These results were further confirmed by confocal fluorescence imaging and statistical analysis of signal intensity for individual cells. From FIG. 36c,d and FIG. 37a, significantly brighter fluorescence signal from the entire cell population is observed over laser treatment time, indicating different levels of membrane permeability. After 10 min laser treatment, such damaged membranes are unable to recover even with 2 hours culturing (FIG. 37b). In contrast, for S. aureus with ΔCrtM (nonpigmented mutant) and log-phase MRSA, no significant difference in SYTOX green uptake is shown between laser treated vs the untreated (FIG. 36e and FIG. 37c).

Based on these findings, we further hypothesize that increased membrane permeability induced by STX photolysis would allow passive diffusion of small-molecule antibiotics that target intracellular activities. To demonstrate this point, we used the aminoglycoside, gentamicin, as an example. Gentamicin was firstly conjugated with a fluorescent dye, Texas red, and then imaged via confocal fluorescence microscopy after co-culturing with cells. As expected, cells with laser treatment accumulate significantly more gentamicin molecules than untreated, from either single cells (FIG. 36f,g) or the entire cell population (FIG. 36d). The uptake of ciprofloxacin, another small-molecule antibiotic that belongs to fluoroquinolone class, can be directly detected via its endogenous fluorescent nature. Compared to the untreated cells, increased fluorescence signal is shown on cells with laser treatment (FIG. 36h). These results further confirm that small-molecule antibiotics can diffuse into the cell via permeable membrane induced by laser treatment.

To estimate how large a molecule can diffuse into the damaged membrane, we applied dextran labeled fluorescein isothiocyanate (FITC-dextran) with variable molecular weight/Stokes radius and monitored its insertion before and after laser treatment. For FD70 with molecular weight of 70 k Da and Stokes radius of 6 nm, longer laser treatment time yields increased fluorescence signal either at individual cell level (FIG. 2i) or from total cell population (FIG. 36j). Laser treatment over 5 min leads to ample insertion of FD 70 (FIG. 36j). Super-resolution imaging of individual cells further shows that these dyes are primarily inserted and concentrated within FMM (FIG. 36i, zoom-in images). In contrast, when FD500 with molecular weight of 500 k Da and Stokes radius of 15 nm was applied, no uptake is shown, indicating an upper limit on molecular Stoke radius of 30 nm level (FIG. 36k). These results suggest that after effective STX photolysis, FMM becomes porous, allowing molecules with Stokes radius up to nanometer level to diffuse through or insert into the membrane.

### Example 3. Photo-Disasembly of Functional Membrane Microdomains: Membrane Fluidification

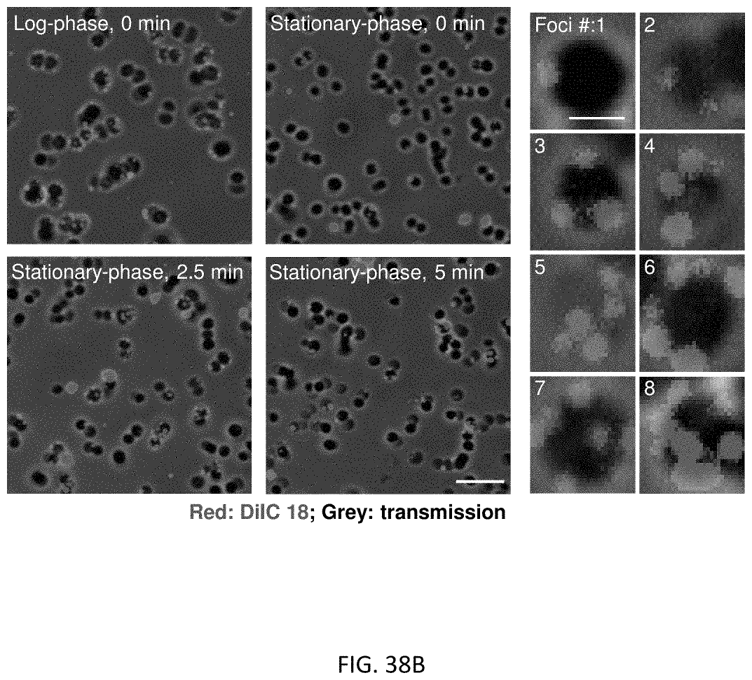

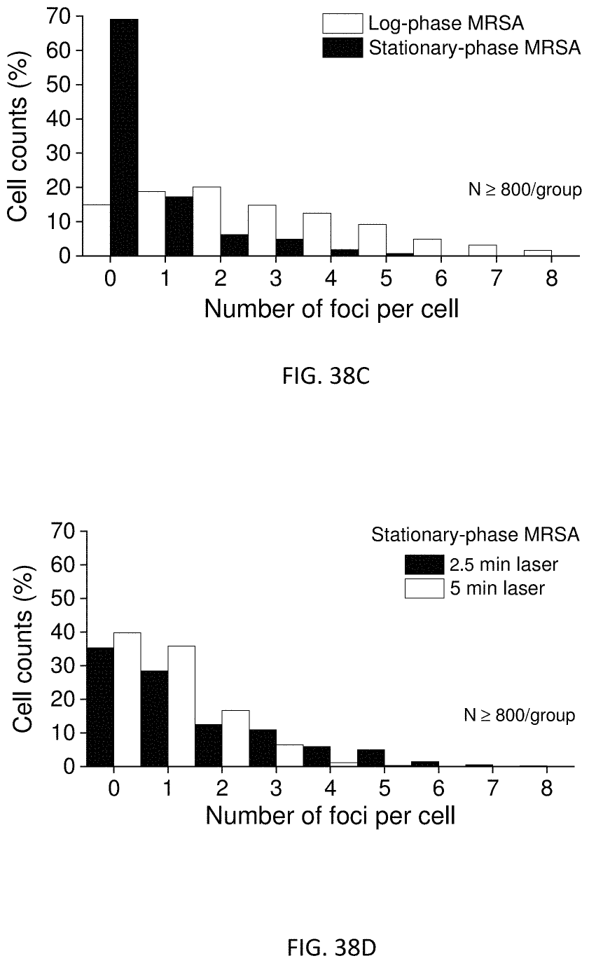

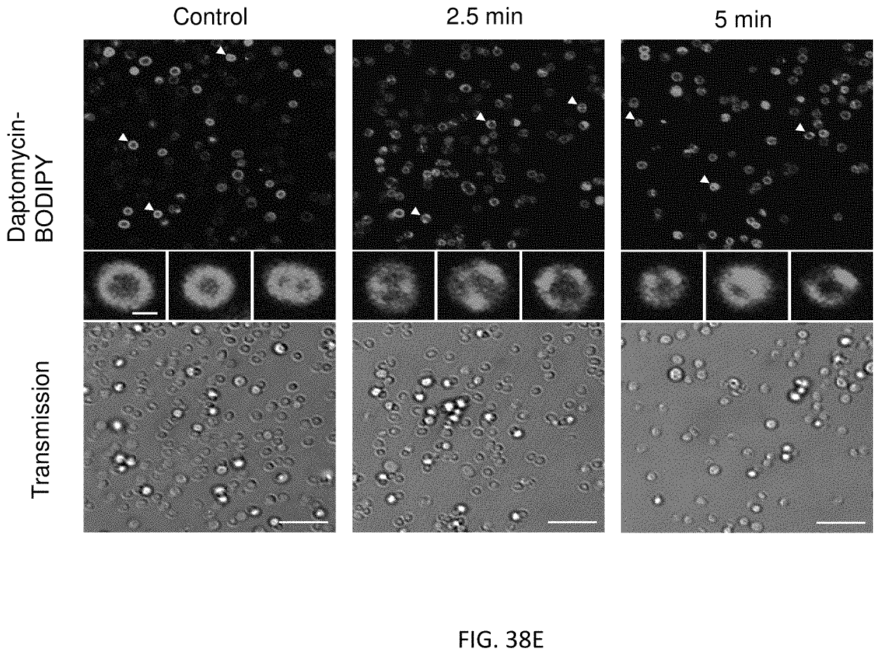

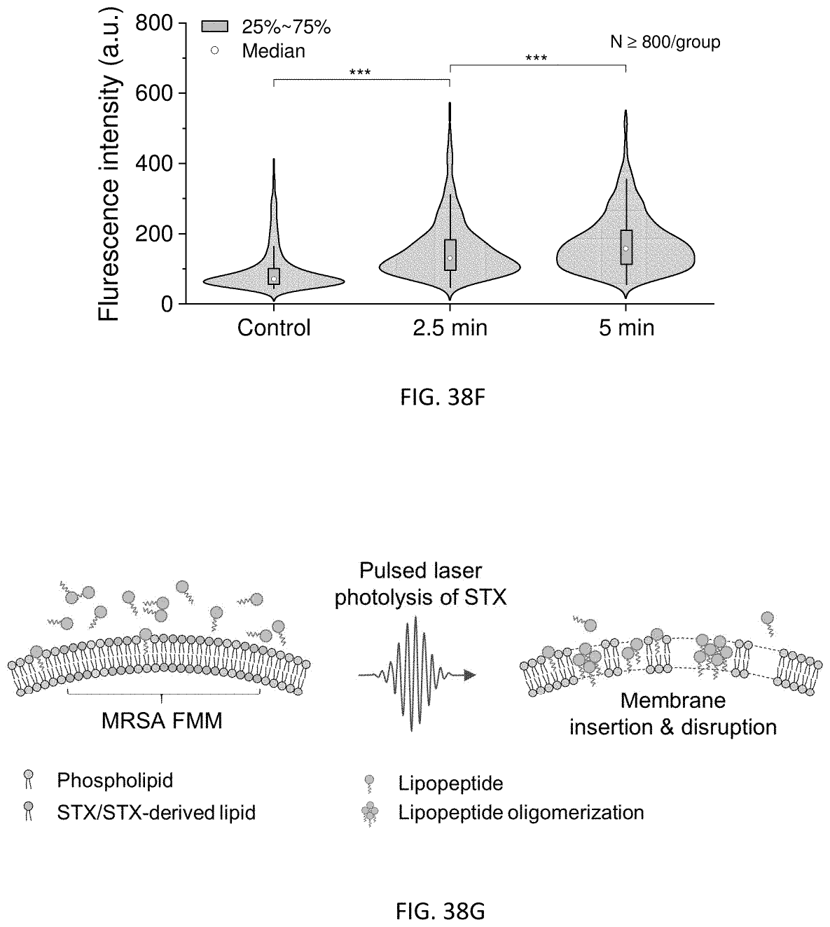

After effective STX photolysis, its products no longer maintain the chemical structure and properties of STX. The unsaturated tail of STX is truncated as unveiled by Raman spectroscopy results; the polarity of its products becomes significantly higher than that of STX as suggested by liquid chromatography results¹⁷. As a result, these products spontaneously tend to disperse or detach from their original membrane organization. These behaviors profoundly disrupt the lipid packing within the microdomain, thus changing the membrane fluidity and subsequently facilitating the insertion of membrane targeting antibiotics, e.g. daptomycin. To test this hypothesis, we evaluated the membrane fluidity with or without laser treatment by DiIC₁₈, a fluorescent dye that displays affinity for membrane areas with increased fluidity due to its short hydrocarbon tail²⁴ (FIG. 38a). As shown in FIG. 38b,c, significantly more DiIC₁₈ is shown up as foci in log-phase MRSA when compared to the stationary-phase, as membrane in stationary phase becomes more rigid than that in log phase, partially due to the presence of rigid STX²⁵. After laser treatment, the foci number on each cell is significantly increased when compared with that of stationary-phase cells without laser treatment. Notably, 70% of cells in stationary phase show no detectable fluorescence signal, whereas this portion drops dramatically to 35% after 2.5 min laser treatment. The ample uptake indicates that laser treatment renders membrane more fluid due to the depletion of rigid unsaturated STX tail and the subsequent loose packing of lipid bilayer.

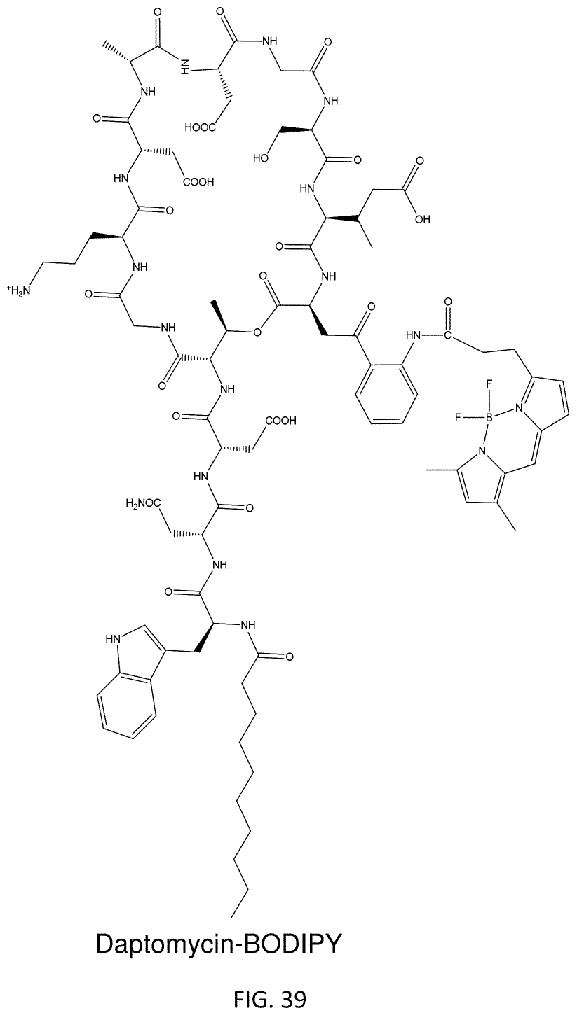

The increased membrane rigidity by STX overexpression promotes the bacterial resistance against daptomycin, a cationic antimicrobial peptide, by reducing its membrane binding and subsequent membrane disruption²⁵⁻²⁷. Therefore, we further hypothesize that increased membrane fluidity after STX photolysis facilitates the insertion of daptomycin. To prove this point, we first labeled daptomycin with BODIPY (molecular structure shown in FIG. 39a), then imaged cellular uptake of daptomycin with or without laser treatment. From FIG. 38e, f, significantly more daptomycin uptake is shown for laser-treated groups when compared to the untreated groups; longer treatment yields higher uptake. More interestingly, daptomycin distribution between laser treated and untreated groups are quite different; for the untreated, daptomycin distributes evenly on the cell membrane, whereas, aggregates or domain-like structures with bright signal are found on cells after laser treatment (representative zoom-in images in the middle row in FIG. 38e). These aggregates most likely form within FMM due to the promoted insertion and oligomerization of daptomycin. Collectively, these results provide evidences to support the ample increase of membrane fluidity after STX photolysis, thus potentiate antibiotic lipopeptides to insert and oligomerize within the domains and further disrupt cell membrane as illustrated in FIG. 38g.

### Example 2. Photo-Disasembly of Functional Membrane Microdomains: Membrane Protein Detachment

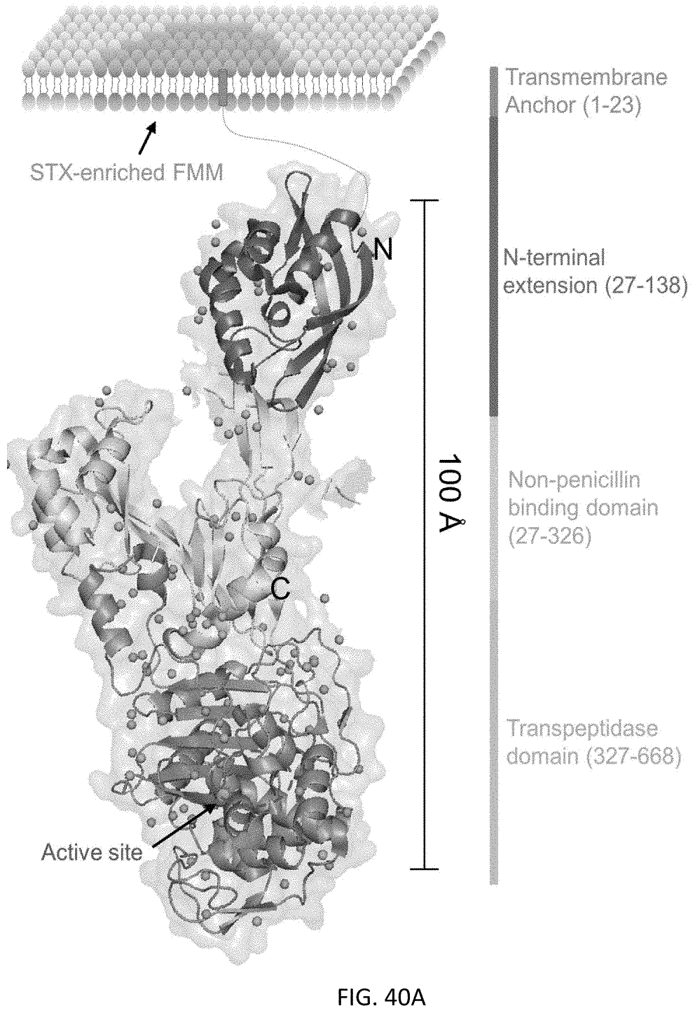

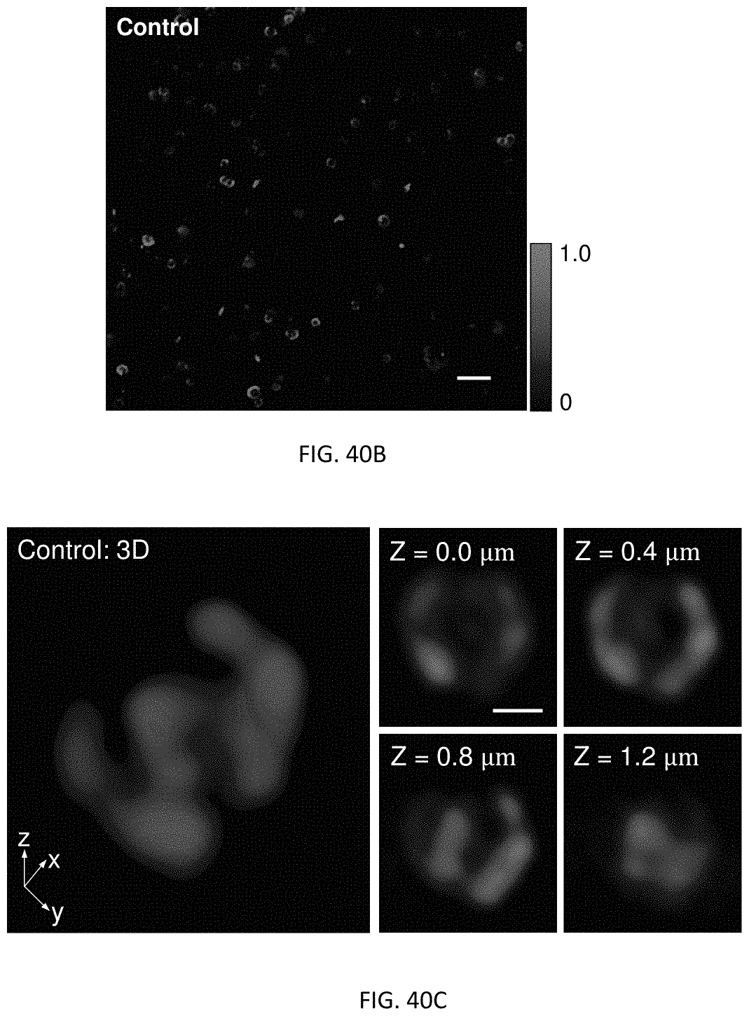

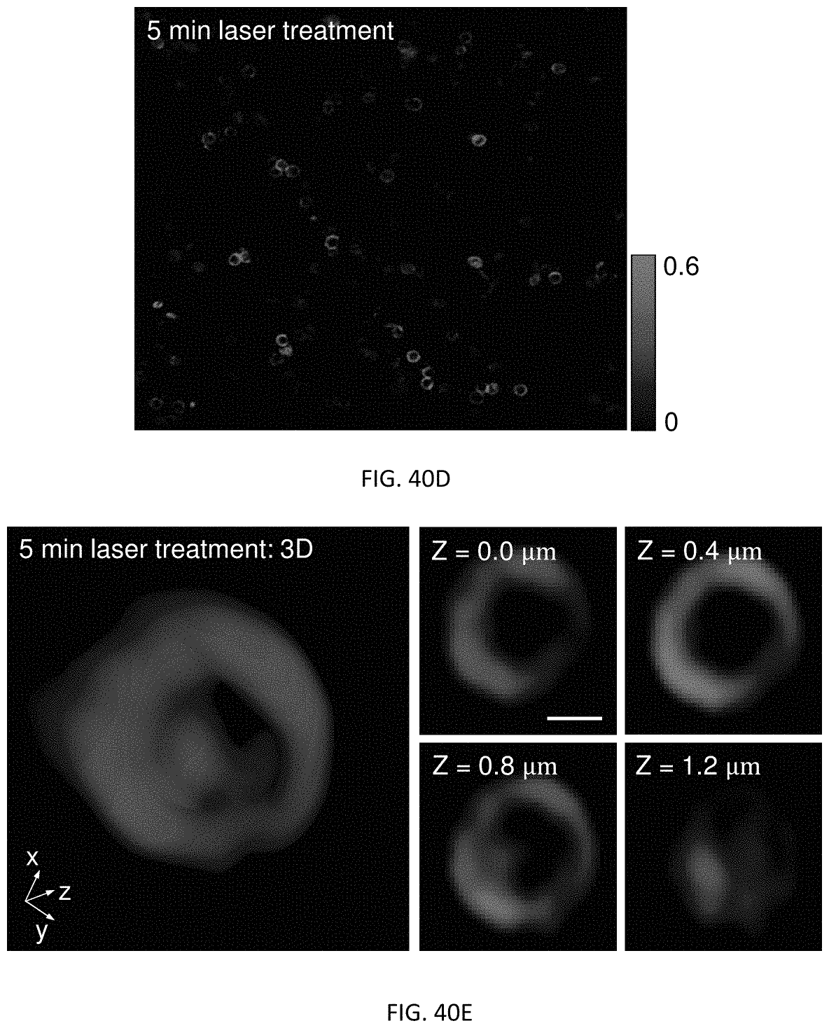

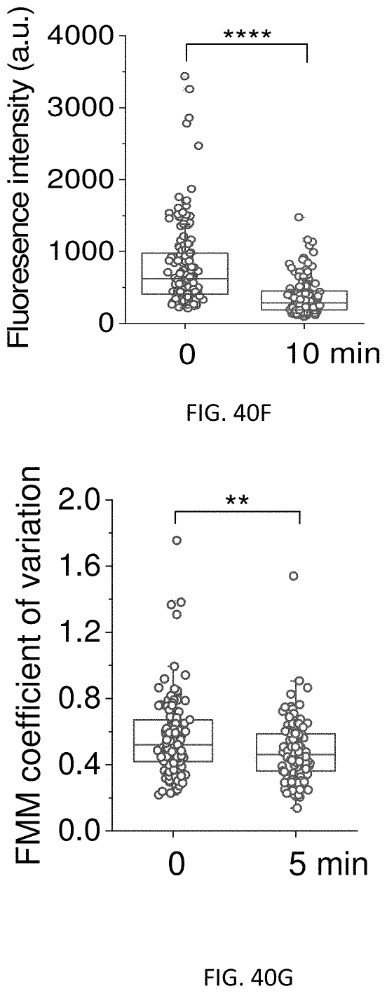

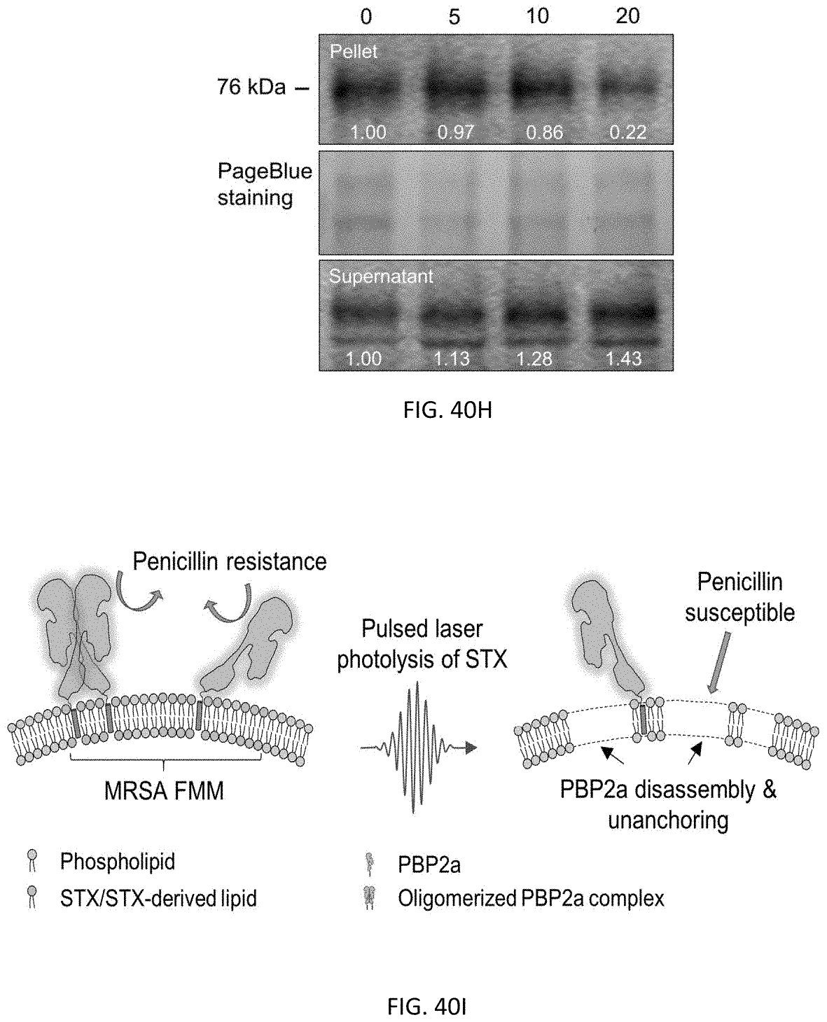

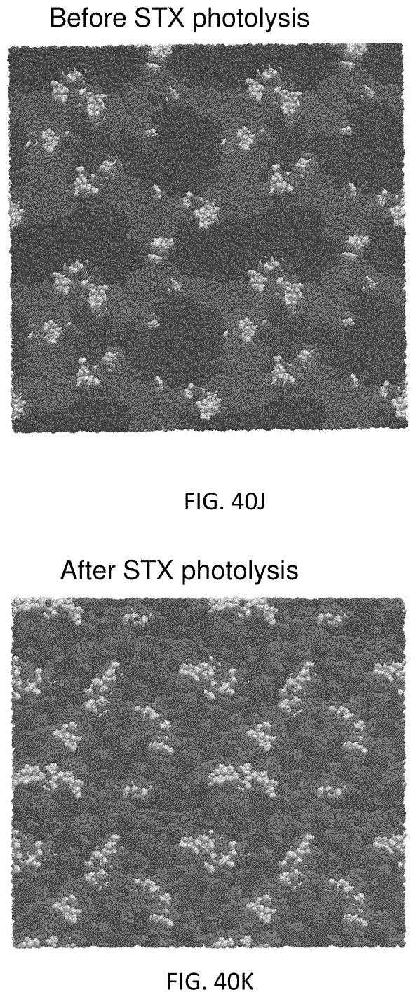

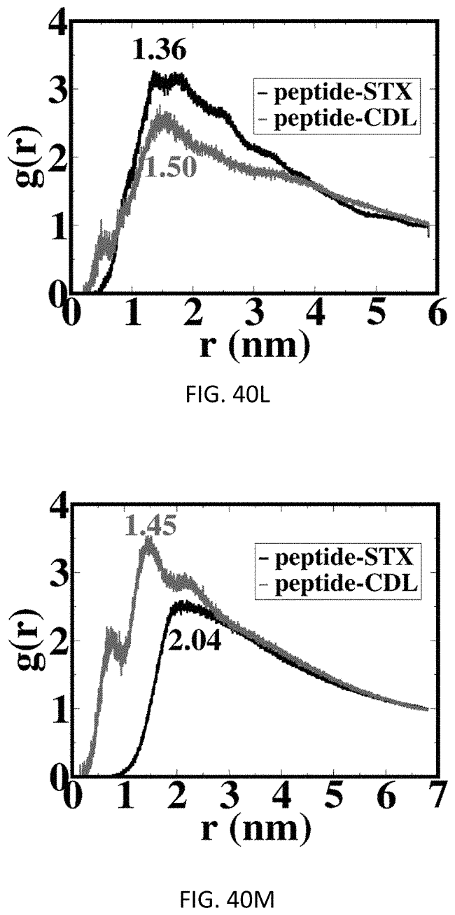

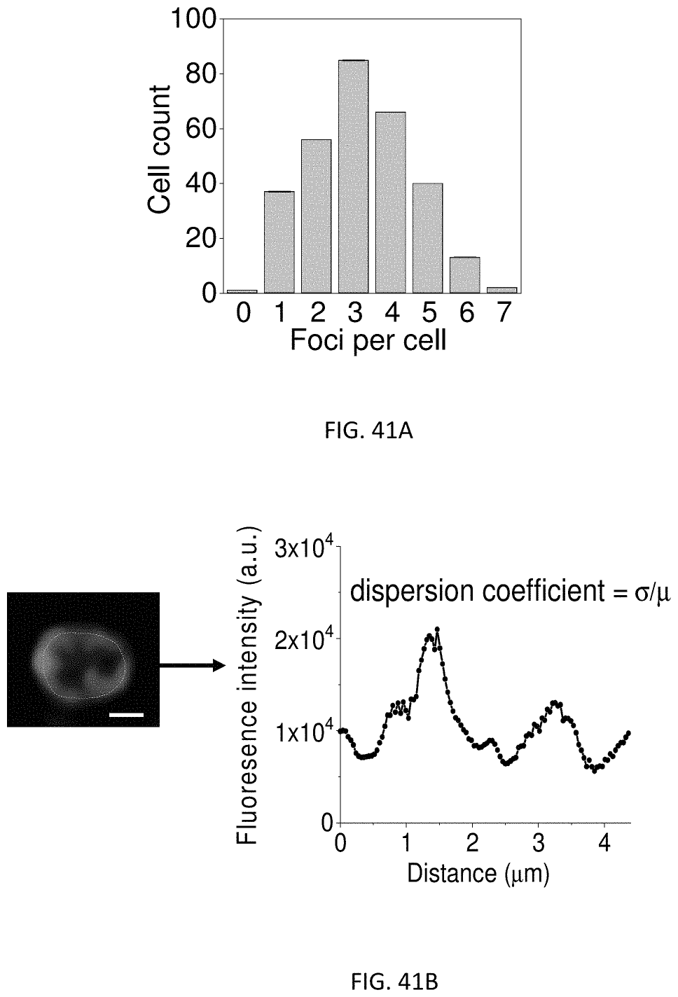

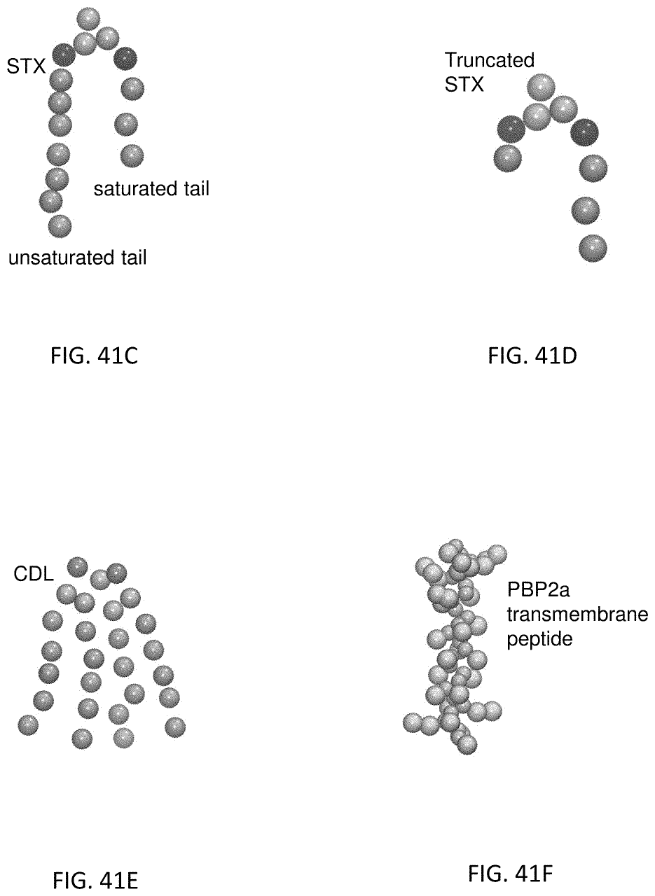

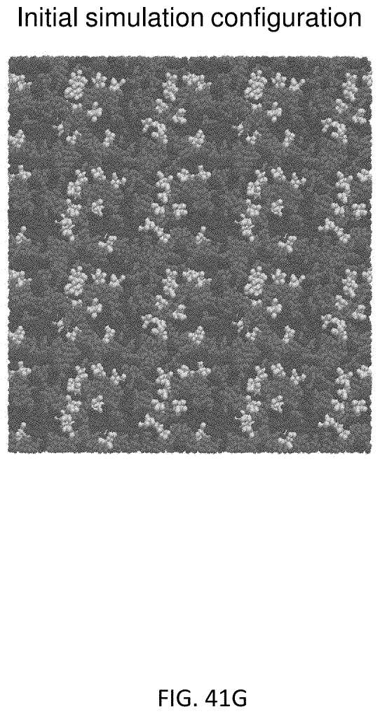

To demonstrate how STX photolysis further malfunctions membrane proteins that are co-localized within STX-enriched FMM, we chose penicillin-binding protein 2a, PBP2a, as an example. MRSA acquires resistance to beta-lactam antibiotics through expression of PBP2a, a protein² that primarily anchors within FMM through its transmembrane helix and hides its targeting site inaccessible by beta-lactam antibiotics (FIG. 40a). Considering the relative structural organization of STX and PBP2a, we hypothesize that PBP2a protein complex can be disassembled and unanchored from cell membrane upon effective STX photolysis. To validate this point, we first resolved the structural distribution of PBP2a under a structured illumination microscopy via immunostaining with anti-PBP2a antibodies both for laser-treated (FIG. 40b,c) and the untreated (FIG. 40d,e). For the untreated, we observed bright fluorescence signal from all stationary-phase MRSA cells due to ample PBP2a expression. These proteins are accumulated discretely within small membrane domains as visualized in both 3-D (FIG. 40b) and 2-D along various depths (FIG. 40c). Three to four foci on average is found on each cell, indicating the prevalence of microdomain formation once cells reached their stationary phase (FIG. 41a). Once treated with pulsed laser, dramatically decreased signal intensity and altered signal distribution are observed on each individual cell (FIG. 40d,e). Laser-treated cells have around 2 times lower signal intensity when compared with the untreated, thus indicating a large portion of PBP2a proteins are detached from cell membrane (FIG. 40f). The left PBP2a proteins are dispersed laterally with its dispersion quantified by coefficient of variation, which is significantly higher than that of the untreated (FIG. 40g, quantification method shown in FIG. 41b). Such detachment and dispersion lead to significantly reduced contrast between FMM and its neighboring lipid bilayer (FIG. 40e). Western blotting results further confirms the PBP2a detachment mechanism, as increased amount of PBP2a is found in supernatant over laser treatment time, whereas decreased amount found in MRSA pellets (FIG. 40h). Taken together, photolysis of the constituent lipids leads to disassembly and detachment of PBP2a from FMM, thus disables MRSA's defense to penicillins as illustrated in FIG. 40i. Additionally, as PBP2a is primarily utilized to catalyze cell-wall crosslinking, their detachment further affects cell wall synthesis and potentially cell viability.

To further investigate the membrane phase and its mechanical properties, we built a coarse-grained membrane model that contains STX, cardiolipin lipids, and transmembrane helixes of PBP2a proteins (coarse-grained representations shown in FIG. 41c-f) and performed microsecond-scale molecular dynamics simulations. At the initial simulation configuration, STX, cardiolipin lipids and peptides randomly disperse in the built bilayer (FIG. 41g). During 10 μs simulation, these molecules spontaneously self-assemble to a microphase separated system containing well distinguishable STX and cardiolipin microdomains, despite cardiolipin being a charged lipid; PBP2a peptides localize to the center of STX domains or the vicinity of STX/cardiolipin domain interface (FIG. 40j). The formation of microdomain is primarily driven by the preferable interactions among lipid tails of similar saturation or unsaturation nature, as in current system all four tails of cardiolipin are saturated, whereas STX lipid has a long unsaturated tail. This result is consistent with lipid domain formation commonly found for systems with a mixture of saturated and unsaturated lipids such as DOPC/DPPC, DOPC/DPPG, DOPG/DPPC and many others²⁸. To quantify the relative position and abundance of PBP2a peptides relative to STX and cardiolipin lipids, the radial distribution functions (RDFs), g(r), of PBP2a peptides were calculated. FIGS. 40L-40M show that the RDF peak of PBP2a peptide to STX is higher and located at smaller distance when compared to that of PBP2a peptide to cardiolipin, indicating that PBP2a peptides preferentially interact with STX lipids over cardiolipin, due likely to the better packing between the rigid fully unsaturated STX tail and the PBP2a transmembrane helix.

Our Raman spectroscopy results suggest that photolysis of STX leads to the loss of its rigid and unsaturated tail, the conjugated C═C chain. Thus, to mimic the scenario after STX photolysis, we repeated our simulations by replacing full-length STX with truncated STX with its unsaturated tail removed from the model (FIG. 41d). Interestingly, the truncated STX lipids no longer form microdomains. As a result, all the lipids and PBP2a peptides are randomly dispersed (FIG. 40k). Moreover, the RDF of PBP2a peptide to cardiolipin now features a higher peak at a smaller distance than that of PBP2a peptide to STX, suggesting that the PBP2a proteins prefer to interact with cardiolipins over truncated STX (FIG. 40l, lower panel). The different phase features before and after STX photolysis also lead to different membrane mechanics. For example, the calculated area expansion modulus (K_{A}) of the membrane after microdomain formation is ˜58 k_{B}T/nm², which is significantly higher than the value of ˜42 k_{B}T/nm² with truncated STX, cardiolipins and peptides randomly dispersed after STX photolysis. This suggests that following the truncation of the unsaturated STX tail, the membrane loses the microphase separated domain structure and becomes more loosely packed, which in turn likely reduces the affinity of PBP2a protein to the membrane. Collectively, our simulations provide a plausible rational for the STX photolysis induced membrane remodeling, including the loss of functional domains, the increase of membrane permeability and fluidity, and the detachment of PBP2a from the membrane.

### Example 5. Potentiation of Conventional Antibiotics

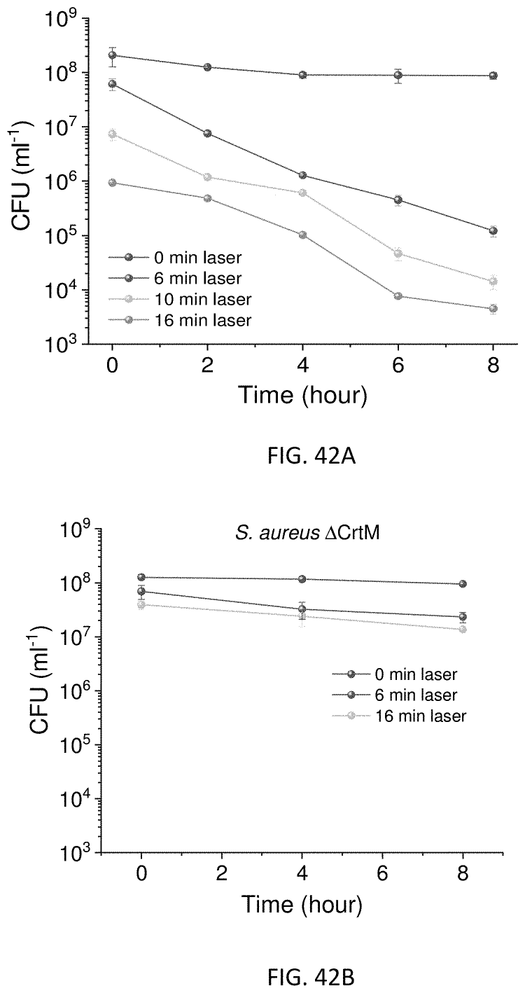

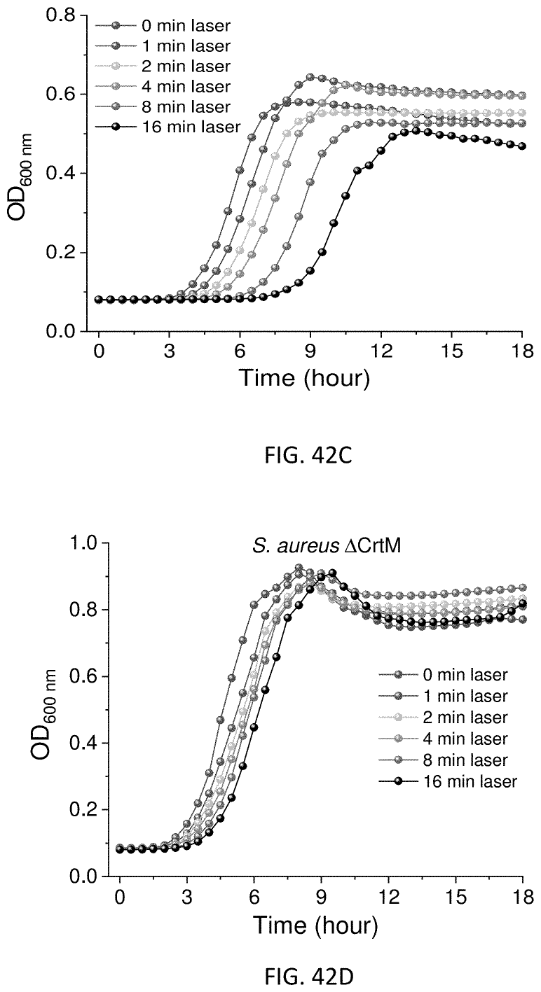

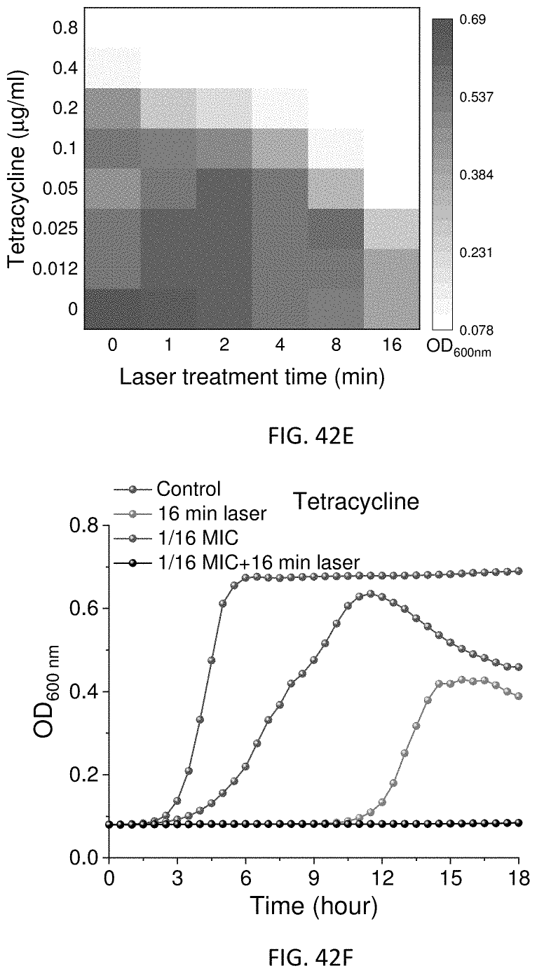

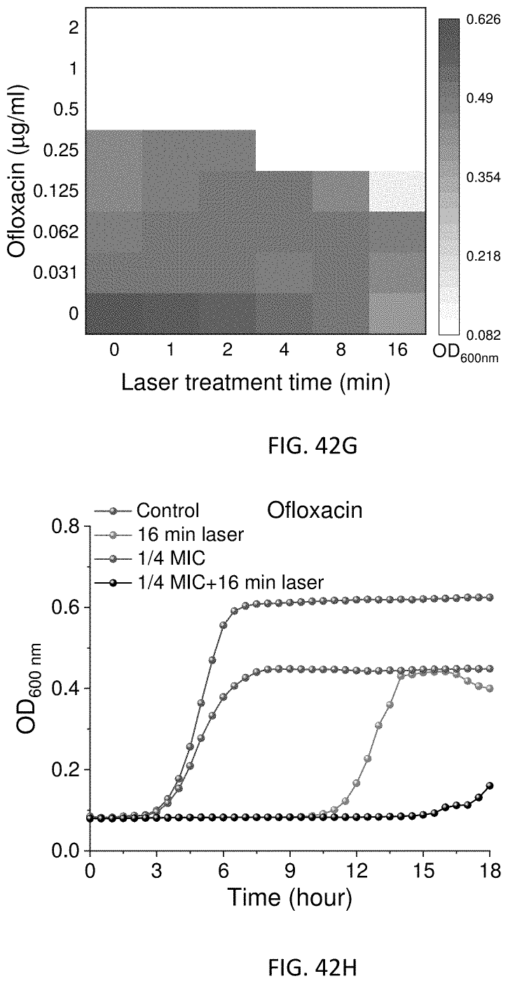

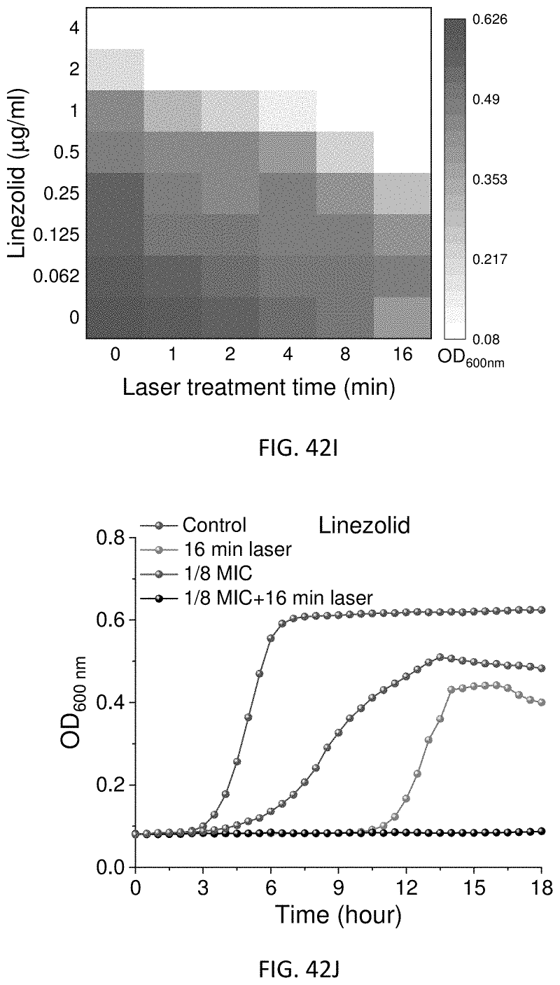

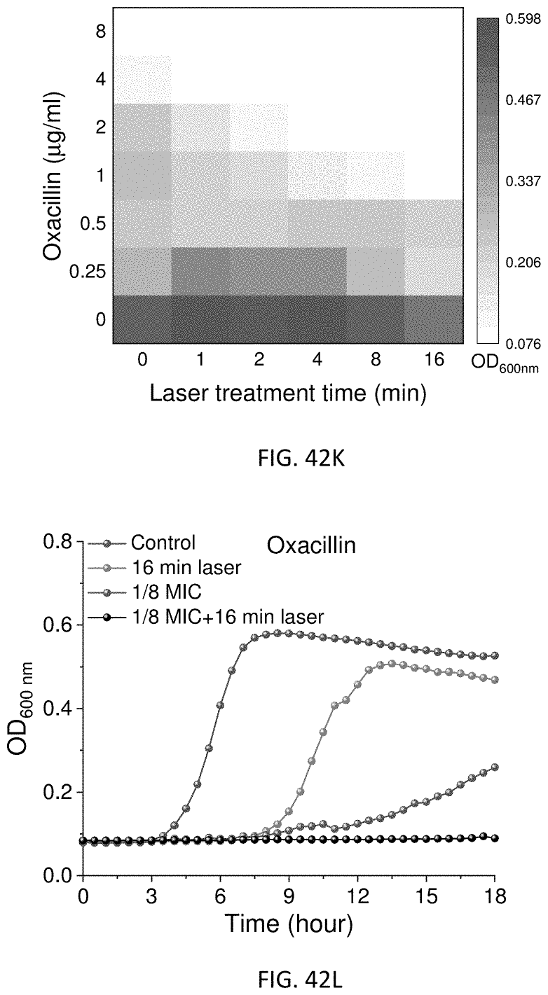

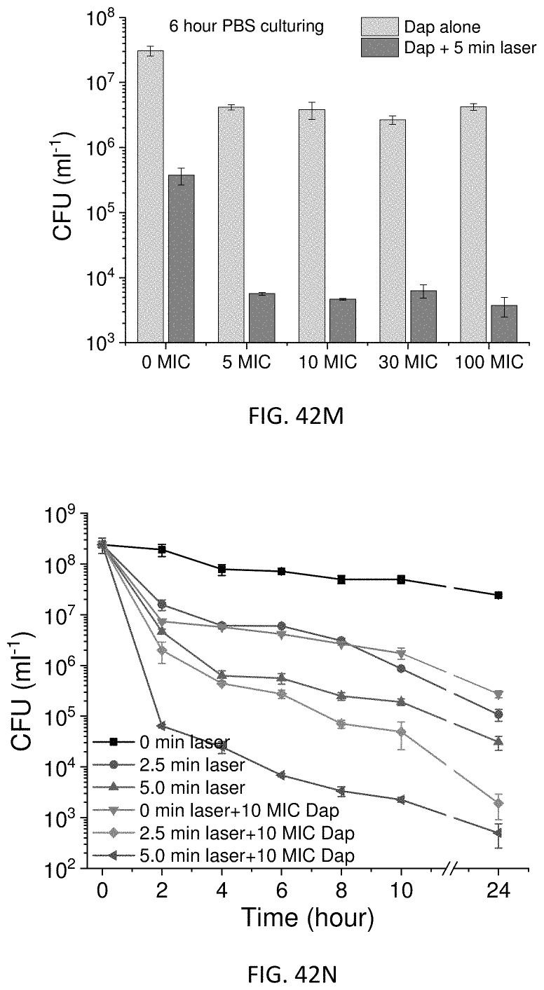

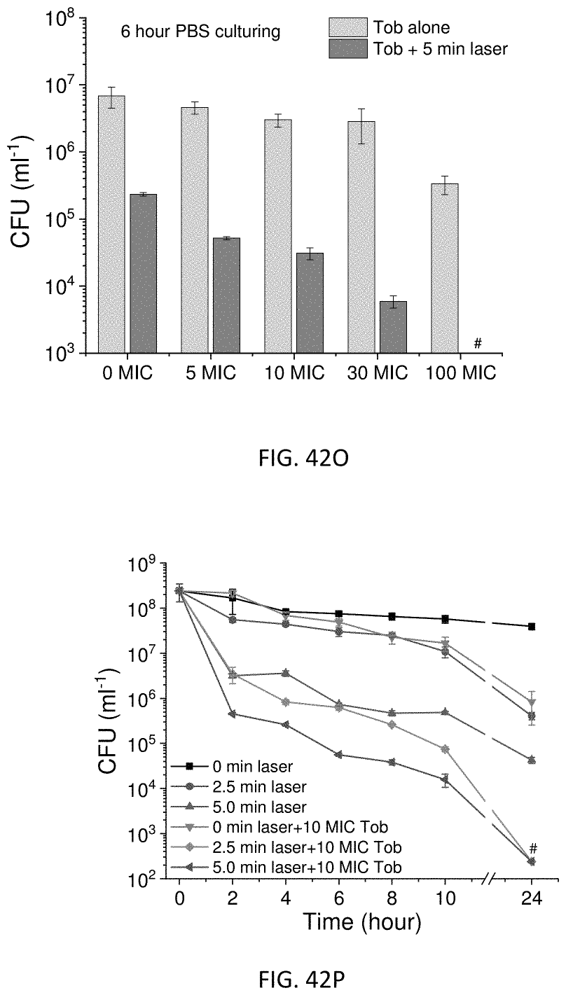

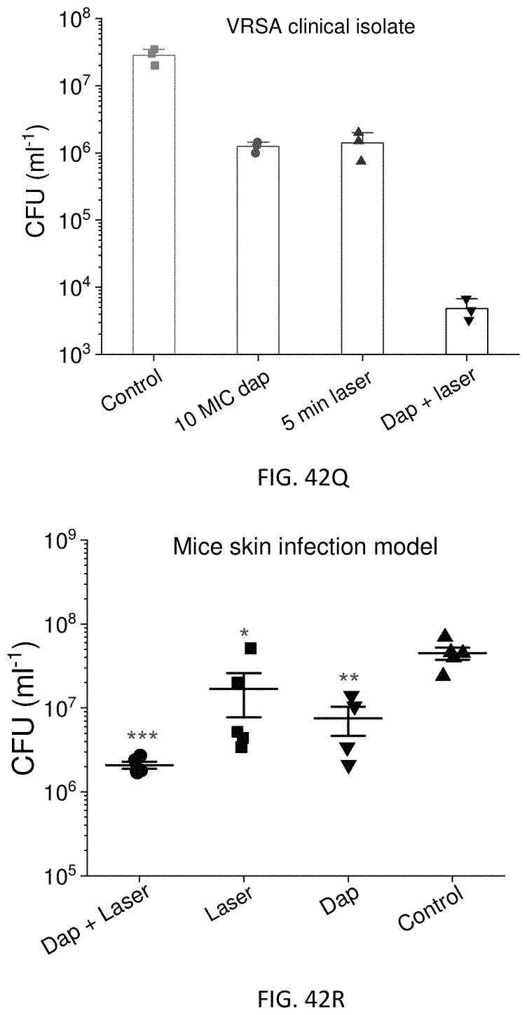

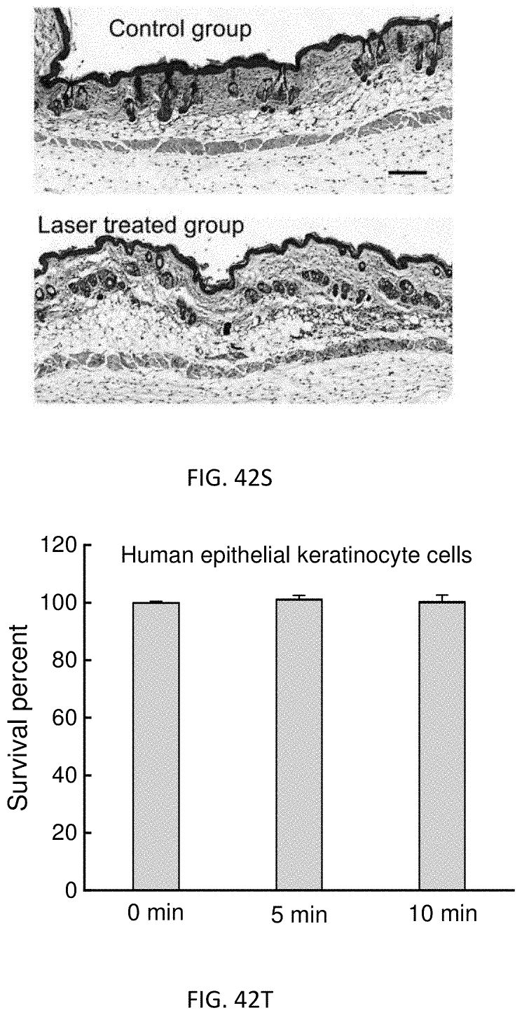

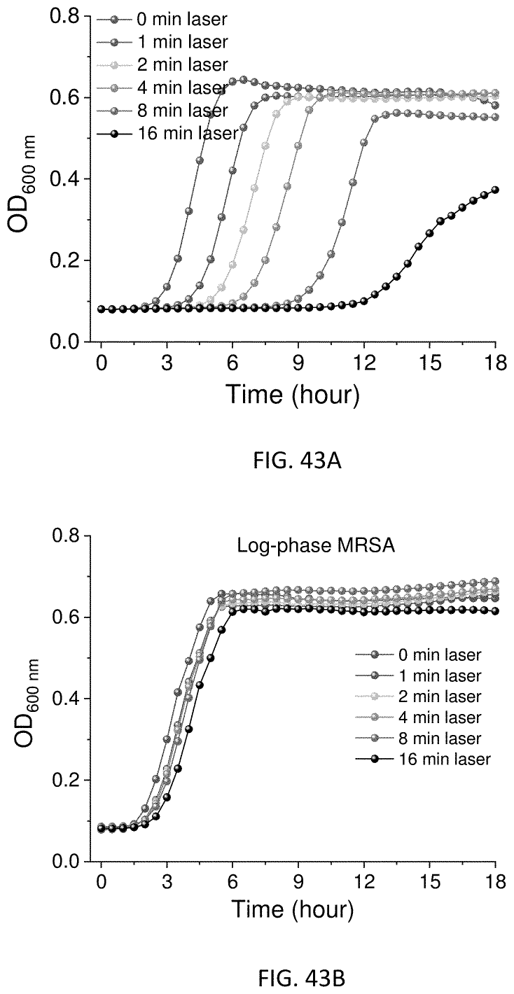

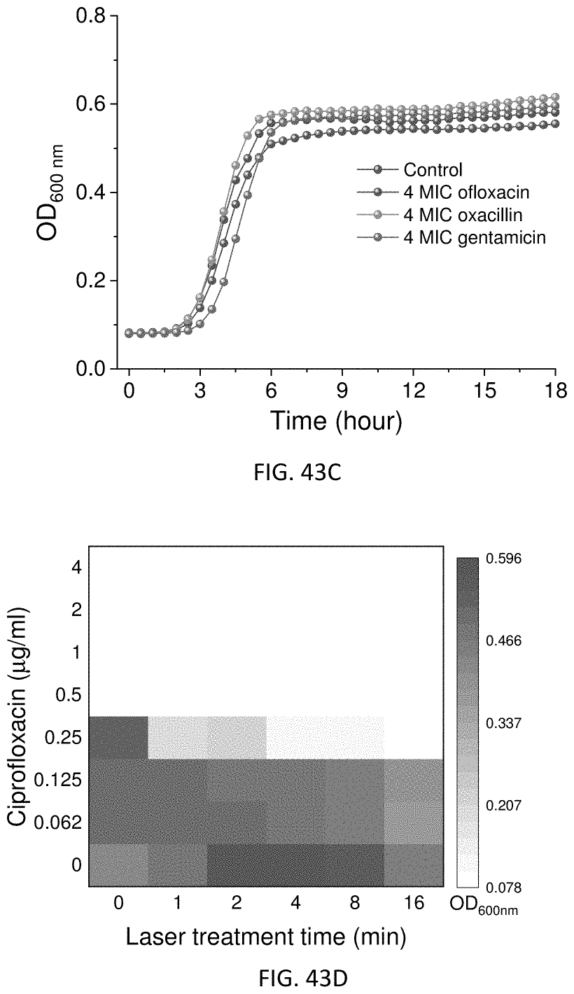

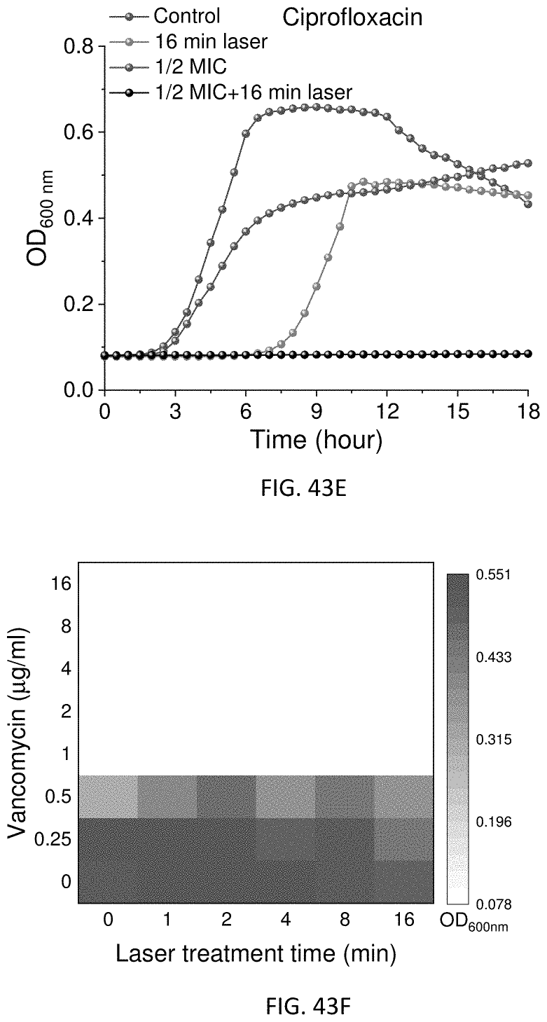

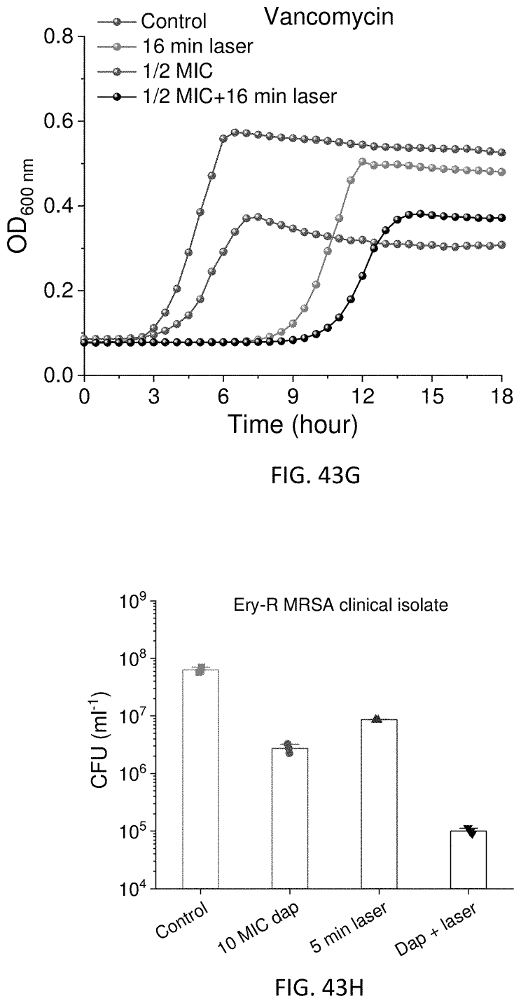

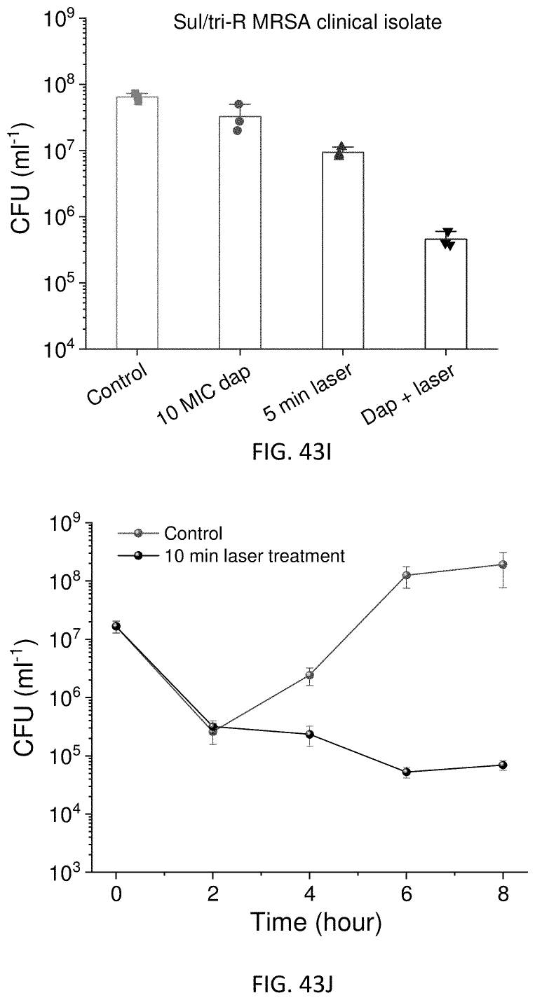

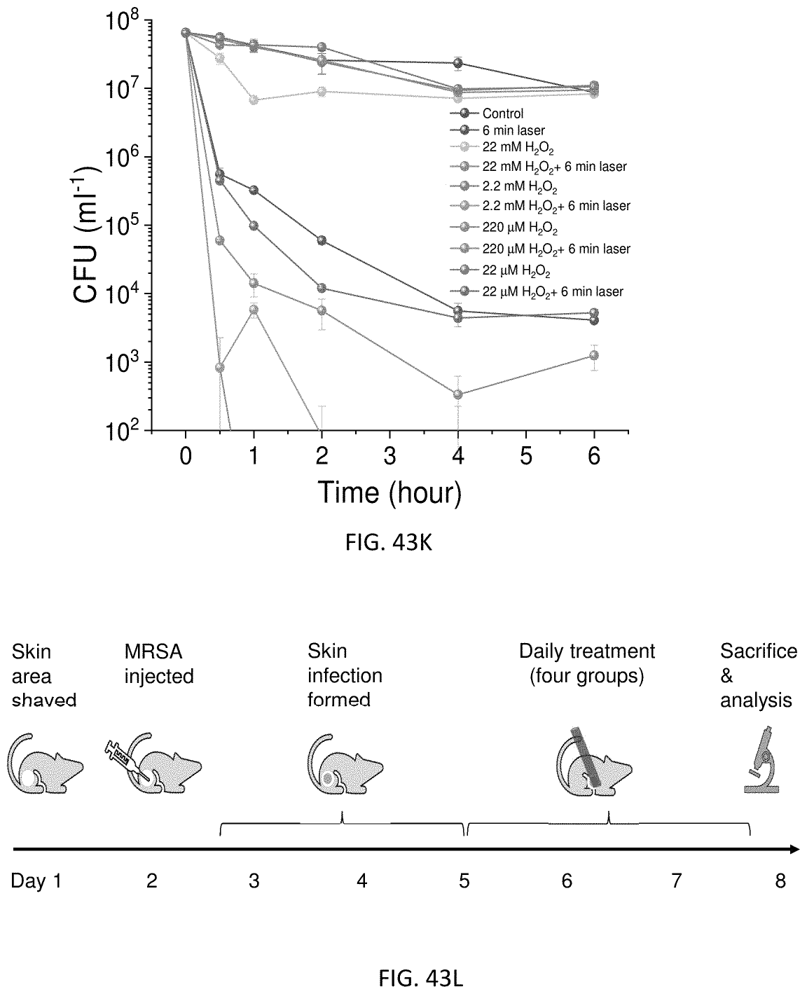

With cell membrane catastrophically damaged via STX photolysis, we further reasoned that both cell growth and cell viability are severely compromised by laser treatment alone. To test this point, time-killing assay in phosphate-buffered saline was firstly performed on stationary-phase cells with or without laser treatment. Compared with the untreated, laser-treated cells are killed quickly and efficiently due to their disassembled FMM and incapacity for recovery (FIG. 42a). The killing efficiency shows strong illumination dosage/time dependence; 16 min laser treatment yields nearly 5-log killing when compared to the untreated. In contrast, S. aureus ΔCrtM shows relatively negligible killing by laser treatment (FIG. 42b). These results confirm that STX photolysis induced membrane disruption is the underlying eradication mechanism. Additionally, its recovery ability after laser treatment was assessed via a post-exposure effect assay, similar to post-antibiotic effect²⁹, as an important way to establish the optimal dosing regimen. The post-exposure effect of stationary-phase MRSA, depending on STX expression condition and laser treatment time, reaches up to 6-9 hours, due primarily to the membrane disruption mechanisms (FIG. 42c and FIG. 43a), whereas no significant post-exposure effect observed for log-phase MRSA (FIG. 43b) or S. aureus ΔCrtM (FIG. 42d). This post-exposure effect indicates a very slow recovery for stationary-phase cells after laser treatment thus fewer doses required for patients, which is superior than the post-antibiotic effect of most antibiotics including oxacillin, ofloxacin, and gentamicin (<1 hour post-antibiotic effect for all three antibiotics, FIG. 43c). More significantly, STX photolysis-induced FMM disassembly can pave a new approach to sensitize these bacteria to conventional antibiotics, even by antibiotics presumed to have no activity against MRSA, such as penicillins.

To demonstrate the laser treatment-mediated synergism with antibiotics, we first applied the checkerboard assay as a screening method. Interestingly, synergism is identified between laser treatment and several major classes of antibiotics for MRSA growth inhibition (FIG. 42e-1). Using tetracycline as an example, the lowest concentration needed to completely inhibit MRSA growth within 18 hours is steadily decreased by elongated laser treatment time; 16 min laser treatment enables a 16-fold reduction, where 2-fold change or larger is regarded as synergy based on fractional inhibitory concentration index (FICI) (FIG. 42e,f). The similar results are found for quinolones: ofloxacin and ciprofloxacin (FIG. 42g,h and FIG. 43d,e) and oxazolidinone: linezolid (FIG. 42i,j) with 2-fold, 8-fold reduction respectively. Notably, tetracyclines, oxazolidinones and quinolones all target intracellular activities; therefore, they have to penetrate through the membrane barrier in order to be functional. These growth inhibition results further validate our hypothesis that photo-disassembly of FMM renders membrane permeable to allow passive diffusion of small-molecule antibiotics inside cells, thus increasing their effectiveness against MRSA. Due to the disassembly and detachment of PBP2a proteins on cell membrane, laser treatment further re-sensitizes MRSA to penicillin: oxacillin with its concentration as low as 1 μg/ml, 8-fold lower than that of oxacillin-treated alone (FIG. 42k,l). In contrast, when vancomycin, an antibiotic that inhibits cell wall biosynthesis, was tested, no synergism is shown (FIG. 43f,g). For bactericidal antibiotics, time-killing assay was then applied as the screening method. Due to laser-mediated membrane insertion and further disruption, 10-minimum inhibitory concentration (MIC) daptomycin is found capable of eradicating stationary-phase/dormant MRSA cells synergistically with only 5 min laser treatment (e.g. more than 3.5-log reduction after 6 hours), whereas antibiotics alone show very limited killing even at 100 MIC (e.g. 1-log reduction after 6 hours) (FIG. 42m,n). The similar synergistic killing is observed for aminoglycoside: tobramycin (FIG. 42o,p) due to its passive diffusion via laser-mediated permeable membrane. The synergistic therapy between 10 MIC daptomycin and laser treatment are also effective in eradicating VRSA and multidrug-resistant MRSA clinical isolates (FIG. 42q and FIG. 43h,i). Additionally, the synergy with laser treatment for MRSA killing is not only limited to conventional antibiotics; laser treatment facilitates human whole blood by killing stationary-phase MRSA for 3 log (FIG. 43j); ROS-producing agents e.g. hydrogen peroxide (at 220 μM low concentration) synergizes with laser treatment and kills stationary-phase MRSA by 4 log within 2 hours, whereas hydrogen peroxide alone shows minor killing even at 22 mM high concentration (FIG. 43k). In these cases, besides membrane disruption mechanisms, the depleted antioxidant function of STX contributes to ROS-based killing, consistent with previous findings.

To determine the clinical relevance of the synergistic therapy between laser treatment and conventional antibiotics, the last-resort antibiotic, daptomycin, was used as the example and further applied on in vivo mice skin infection models. To compare the efficacy of different treatment schemes, four groups (control group,10 mg/ml daptomycin-treated group, 10 min laser-treated group, and 10 mg/ml daptomycin plus 10 min laser-treated group) were applied following a 4-day treatment protocol as designed in FIG. 43l. After the treatment regimen, infected tissue for each mouse was collected with bacterial load quantified via colony-forming unit (CFU) enumeration. The CFU statistical results for each treatment group (FIG. 42r) suggest that laser alone-treated group and daptomycin alone-treated group enable 58% and 81% cell killing, respectively; whereas daptomycin plus laser treatment kills around 95% of MRSA in infected skin area. Additionally, the wound areas treated by laser plus daptomycin appear healthier and show the trend of recovery when compared to other groups, as these wound areas show significantly less purulent material, swelling and redness around the edge of the wound. To further evaluate the potential phototoxicity in in vivo model, we followed the same treatment protocol as mice skin infection model except removing the MRSA injection step. After the treatment, the skin regions of interest were collected and analyzed via hematoxylin and eosin stained histology slides (representative images shown in FIG. 42s). As expected, no phototoxicity induced structure change is observed in the laser-treated group. Additionally, the viability of human epithelial keratinocyte cells is also not affected by laser treatment, even under high laser dosage (FIG. 42t). Notably, laser dosage applied in these studies is below the ANSI safety limit for human skin exposure at 460 nm²³.

### Example 6. Inhibition of Resistance Development For Conventional Antibiotics

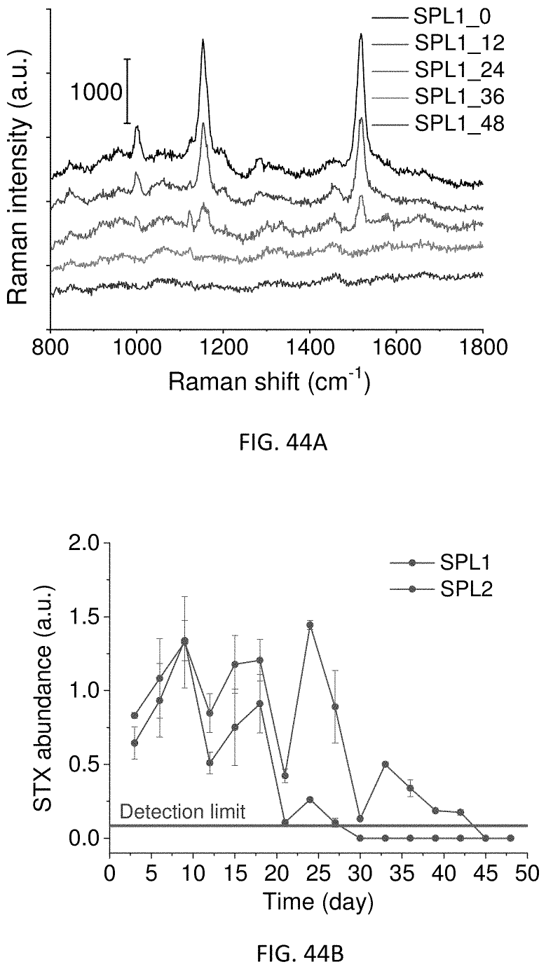

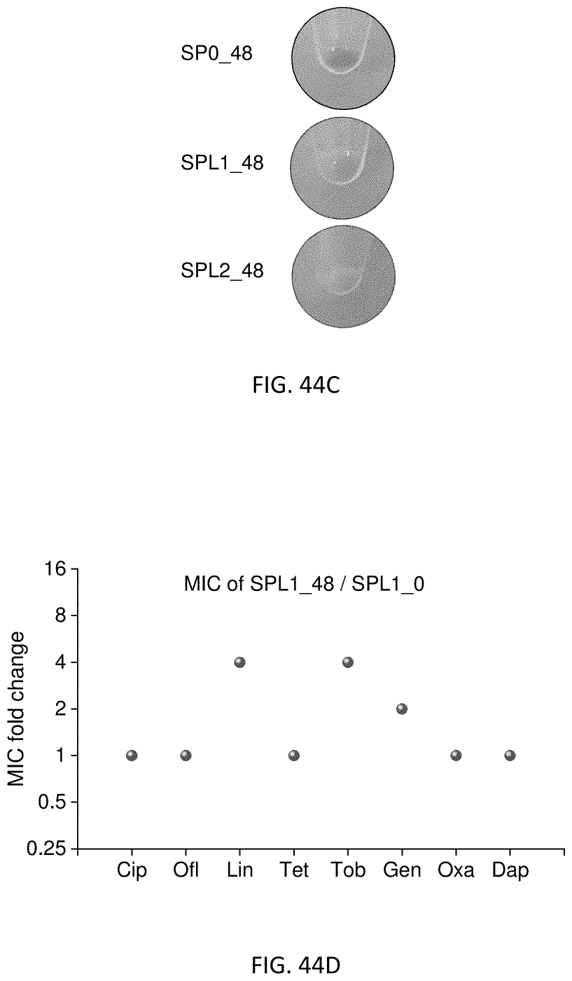

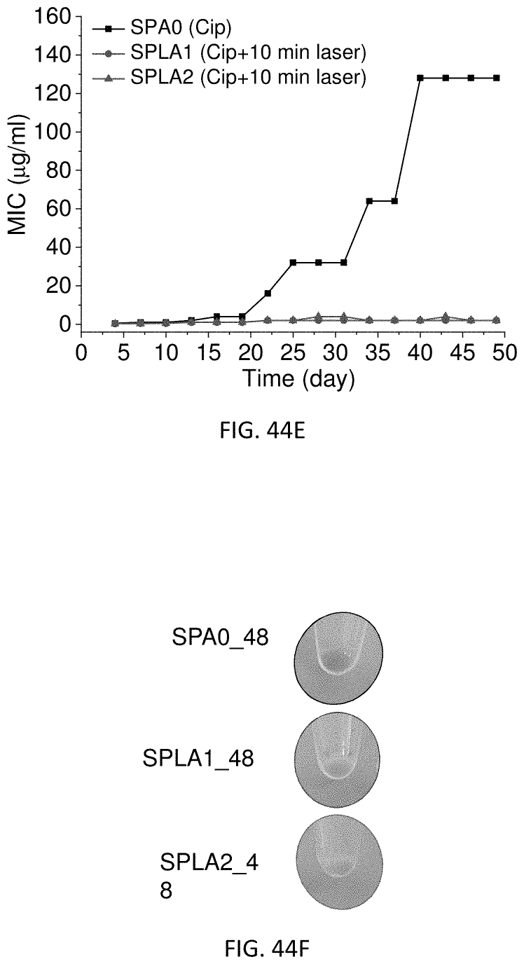

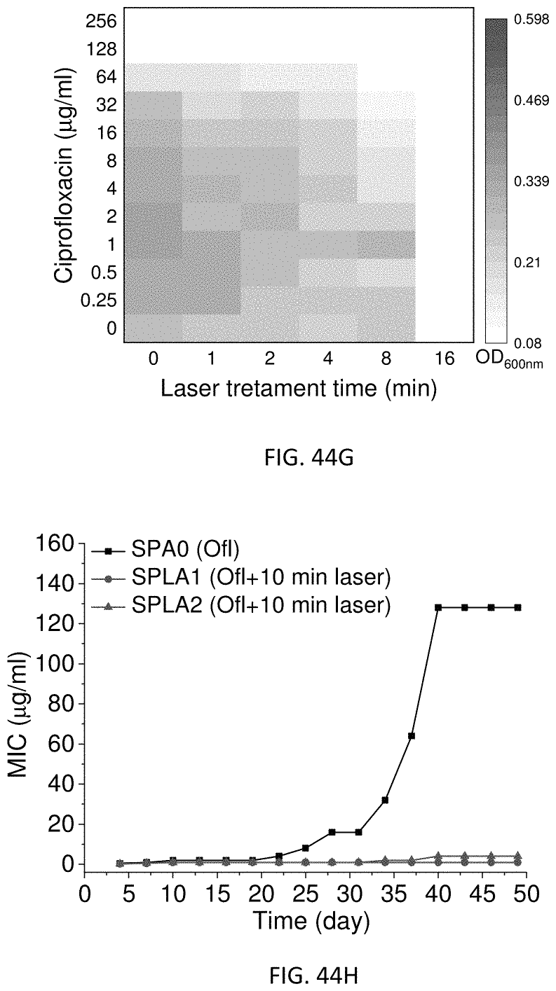

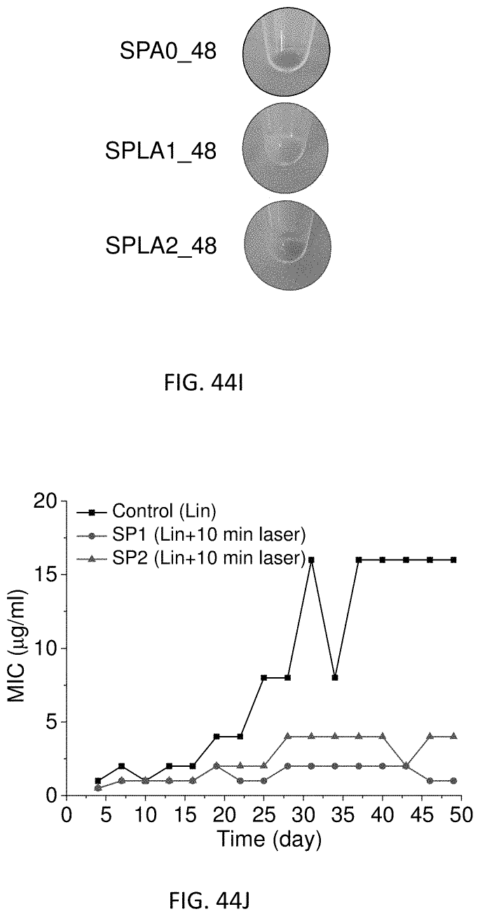

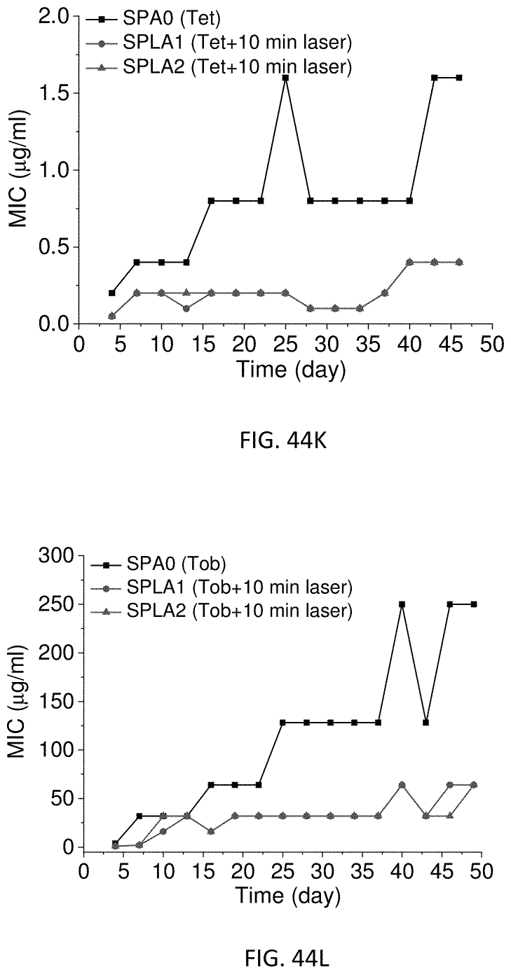

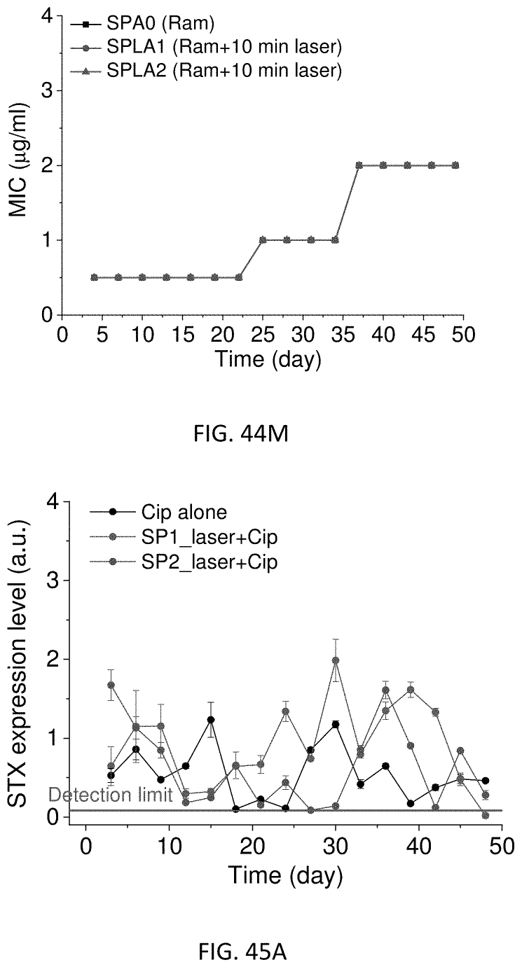

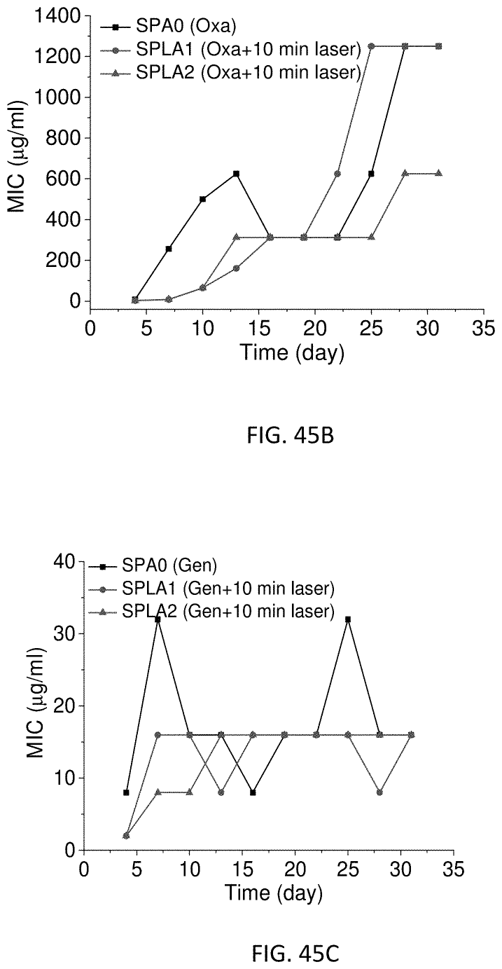

To study MRSA response to our phototherapy, we monitored STX expression level during 48-day serial passage study for 10 min laser alone-treated group. Over the course of 48-day passage, steadily decreased STX expression is observed for laser alone-treated group, as resonance Raman peaks for STX drops over serial passage (FIG. 44a,b); on 30^{th} and 45^{th} day for two independent replicates, STX abundance drops below the detection limit (FIG. 44b); the color of the spun-down cells for both replicates turns to purely white on 48^{th} day whereas the color of the untreated kept golden (FIG. 6c). Plate inoculation results further confirm that there is no single colony expressing STX pigment for both replicates after 48-day passage. These results suggest that STX virulence can be eliminated by serial laser treatment without any resistance development. When compared with the original MRSA, the susceptibility of this new phenotype to different antibiotics shows no change or only minor change after serial treatment (FIG. 44d). The development of resistance for different antibiotics with or without 10 min laser treatment was studied in parallel by monitoring MICs for each group in the presence of corresponding antibiotic at sub-MIC level over the course of 48-day passage. Strikingly, with the presence of laser treatment, ciprofloxacin-treated group shows no resistance over the entire passage study, as its MIC is kept≤2 μg/ml, whereas the MIC of ciprofloxacin alone-treated group has reached 128 μg/ml, 256-fold increase relative to its starting MIC (FIG. 44e). Its spun-down cells turn purely white for both replicates (FIG. 44f); plate inoculation results show that one replicate has no STX expression and the other with mixture of golden and white colonies, consistent with STX expression level monitored through resonance Raman spectroscopy (FIG. 45a). These results suggest that STX virulence is closely related to ciprofloxacin resistance development via overexpression of efflux pumps³⁰; depletion of STX completely inhibits ciprofloxacin resistance. Therefore, it is highly possible that efflux pump proteins are also co-localized within STX-enriched FMM; STX photolysis malfunctions these efflux pumps while allowing passive diffusion of ciprofloxacin into the cells. Interestingly, using checkerboard assay on the ciprofloxacin-resistant MRSA (MIC: 128 μg/ml), we found that 16 min laser treatment alone completely inhibits the growth of these cells (FIG. 44g). This phenomenon suggests that the survival of ciprofloxacin-resistant MRSA relies heavily on STX expression to promote efflux pumps. To further explore this class of antibiotics, ofloxacin was investigated in the serial passage study. Similar results are achieved as shown in FIG. 44h,i. After a 48-day serial passage, MIC of ofloxacin alone-treated group reaches 128 μg/ml, whereas ofloxacin plus laser-treated replicates have MICs of 1 and 4 μg/ml, respectively. Based on plate inoculation results, one replicate has pure white colonies and the other had a mixture of white and golden colonies. These results suggest STX photolysis not only increases the susceptibility of MRSA to fluoroquinolones, but also inhibits its resistance development.

Subsequently, laser treatment-mediated resistance inhibition is also found for other antibiotic classes previous found to synergize with STX photolysis, including linezolid, tetracycline, and tobramycin (FIG. 44j-l). Delayed resistance development is shown for oxacillin and gentamicin during early serial passages (FIG. 45b,c). In contrast, decreased resistance development is not shown for ramoplanin, a drug that targets cell wall biosynthesis (FIG. 44m), as it is not closely related to the membrane disruption mechanisms. Collectively these results further unveil the causality between STX virulence and antibiotic resistance, as well as demonstrating a way to inhibit resistance development to several major classes of antibiotics via photo-disassembly of FMM.

Material and Methods

### 1. Nanosecond Pulsed Laser and LED Systems

The nanosecond pulsed laser system was composed of a nanosecond pulsed laser source (Opolette HE355 LD, OPOTEK Inc.), a 1 mm-core multimode fiber for light delivery (NA=0.22, OPOTEK Inc.), and a custom-built handheld device. Key specifications of the laser source: tunable wavelength range, 410-2400 nm; pulse repetition rate, 20 Hz; maximum pulse energy at 460 nm, 8 mJ; pulse duration, 5 nanoseconds (ns); spectral linewidth, 4-6 cm⁻¹; pulse-pulse stability, <5%. Within the handheld device, a collimation lens (LB1471-A, Thorlabs) was applied to expand the output beam with a diameter of 1 cm. This device was mounted on a stable optical table for experiments shown in FIG. 34a. After collimation by all these optical components, this system provides a final maximum output of 120 mW (6 mJ in pulse energy). Within the illumination area, photon density follows a near-Gaussian distribution. With the diameter of sample droplet at around 5 mm, the photon density over the sample droplet in this study was assumed uniform.

The continuous-wave LED system applied in this study was composed of a blue light LED (M470L3, Thorlabs), an adjustable collimation adapter (SM2F32-A, Thorlabs), and a power controller (LEDD1B, Thorlabs). The output of the blue light LED is centered at 465 nm with bandwidth of 25 nm and maximum power of 650 mW. The output power of the LED system was adjustable, and its beam size was controlled through the collimator and an iris. In order to compare with nanosecond pulsed laser, the output power of the LED was set to 120 mW and used to illuminate an area of 1 cm in diameter.

### 2. Bacterial Strains and Growth Conditions

Methicillin-resistant S. aureus (MRSA USA 300, NRS 384), S. aureus ΔCrtM mutant, vancomycin-resistant S. aureus (VRSA 9, NR-46419), Methicillin-resistant S. aureus (MRSA USA 500, NRS 385).

Log-phase and stationary-phase bacterial inoculum preparation: colonies from streaked plate of frozen bacterial stock were inoculated in sterile tryptic soy broth (TSB, 22092, Sigma Aldrich) medium and grown in an orbital incubator (12960-946, VWR) with a shaking speed of 200 rpm for 2-3 hours at 37° C. for log-phase bacteria (˜10⁷ cells/ml). Before each experiment, bacterial cells were spun down and then the harvested bacteria pellets were washed with 1×phosphate-buffered saline (PBS) twice and then resuspended in 1×PBS at its original concentration. Stationary-phases bacterial solution were prepared following the same procedure except that bacteria inoculum was cultured to three days.

### 3. Antibiotics and Chemicals

Antibiotics used in this study: daptomycin (103060-53-3, Acros Organics), oxacillin (28221, Sigma Aldrich), gentamicin (G1914, Sigma Aldrich), tobramycin (T4014, Sigma Aldrich), ciprofloxacin (17850, Sigma Aldrich), ofloxacin (08757, Sigma Aldrich), linezolid (PZ0014, Sigma Aldrich), tetracycline (87128, Sigma Aldrich), ramoplanin (R1781, Sigma Aldrich), vancomycin (V2002, Sigma Aldrich). 10 mg/ml stocks of all compounds were made in 1×PBS or DMSO (W387520, Sigma Aldrich) or sterile water. For treatments with daptomycin, sterile medium or buffer was supplemented with CaCl₂ (C79-500, Fisher Scientific) with final working concentration of 50 μg/ml.

Fluorescent dyes used in this study: SYTOX green (S7020, Thermo Fisher Scientific), Texas red-X, succinimidyl ester, single isomer (T20175, Thermo Fisher Scientific), FITC-dextran (FD4, FD70, FD500, Sigma Aldrich). DiIC₁₈ (1,1′-Dioctadecyl-3,3,3′,3′-Tetramethylindocarbocyanine Perchlorate, D282, Thermo Fisher Scientific). BODIPY FL, STP ester, sodium salt (B10006, Thermo Fisher Scientific).

### 4. Resonance Raman Spectroscopy

STX was quantified by its Raman peak amplitude at 1161 cm⁻¹ measured by resonance Raman spectroscopy (1221, LABRAM HR EVO, Horiba) with a 40×objective (Olympus) and an excitation wavelength of 532 nm. Samples (either from bacterial colony or STX extract solution) were sandwiched between two glass cover slides (48393-230, VWR international) with a spatial distance of ˜80 μm. To study staphyloxanthin photolysis kinetics, the same samples were measured after each laser treatment.

### 5. Staphyloxanthin Extraction Protocol

The STX extraction protocol was adapted from a previous report¹². Briefly, 2 ml of stationary-phase MRSA were spun down, washed with 1×PBS. Then the MRSA pellets were harvested through centrifuge and crude STX pigment was extracted by 200 μl warm methanol in dark at 55° C. for 20 min.

### 6. Absorption Spectroscopy

Absorption spectroscopy of MRSA solution was performed after different laser treatment time. Briefly, stationary-phase MRSA stationary-phase MRSA (˜10⁸ cells/ml) was washed and suspended into 1×PBS at its original concentration. Aliquots of 100 μl was transferred into a 96 well plate. The absorption spectrum of the lidded wells after each laser treatment (1.5 min laser treatment interval) were monitored by a plate reader (SpectraMax i3×, Molecular Devices) with a spectral window of 300-800 nm and a step size of 2 nm. For the treatment, each well was directly illuminated by laser beam from the well top (1 cm diameter illumination area, 120 mW). Three independent replicates were applied in the study.

### 7. Fluorescence Microscopic Imaging Techniques

For super-resolution imaging, we used a structured illumination microscope (ELYRA super-resolution microscope, Zeiss) with a 100×oil objective. There are several diode lasers used as the excitation sources in the system (405 nm, 488 nm, 561 nm, 638 nm). In the case of FITC-dextran and PBP2a immunofluorescence imaging, we used excitation wavelengths of 488 nm and 561 nm, respectively. Image processing and analysis were directly performed with the provided software for the system.

For the confocal laser scanning microscope, we used a laser scanning confocal microscope (FV3000, Olympus) with two high-sensitivity GaAsP/GaAs photomultiplier tubes (PMTs). The images demonstrated in this study were acquired in a high-speed resonant Galvo-Galvo scanning mode and via an UPLSAPO 100×oil objective (NA=1.35, Si oil immersion, 0.2 mm working distance). Inside this confocal microscope, there are six solid state diode lasers (405 nm, 445 nm, 488 nm, 514 nm, 561 nm, 640 nm). In the case of SYTOX green, FITC-dextran dyes, and daptomycin-BODIPY, we used an excitation wavelength of 488 nm. For the DiIC₁₈, we used 561 nm as the excitation wavelength. Gentamicin-Texas red was excited by a 514-nm laser.

### 8. SYTOX Green Membrane Permeability Assay

Briefly, 1 ml of stationary-phase MRSA (˜10⁸ cells/nil) was spun down, got rid of the supernatant, and resuspended with 100 μl of sterile 1×PBS. 5 μl of the above solution was then exposed into laser beam with different treatment time (laser power, 120 mW; illumination area, 1 cm in diameter). After treatment, MRSA solution was collected into 985 μl of sterile water, as SYTOX green shows best performance in buffers without phosphate. Subsequently, 10 μl of stock SYTOX green solution (5 mM in DMSO) was supplemented before aliquoting into a 96-well plate. The fluorescence emission intensity at 525 nm (excitation at 488 nm) was monitored by a plate reader (SpectraMax i3×, Molecular Devices) for more than 2 hours with a 5-min interval at room temperature. To further visualize the uptake of SYTOX green under a laser scanning confocal fluorescence microscopy, MRSA cells were further prepared following these steps: spin down MRSA pellets, get rid of the supernatant, wash the pellets with sterile water twice, and fix them with 10% formalin (HT501128-4L, Sigma Aldrich). All experiments were conducted in duplicate or triplicate.

### 9. FITC-Dextran Membrane Permeabilization Assay

To estimate how large a molecule can diffuse into the damaged membrane, we applied dextran conjugated with fluorescein isothiocyanate (FITC-dextran) with variable molecular weight/Stokes radius (FD4-FD500, Sigma Aldrich) and monitored their insertion before and after laser treatment. Briefly, 1 ml of stationary-phase MRSA (˜10⁸ cells/ml) was spun down, got rid of the supernatant, and resuspended with 100 μl of sterile 1×PBS. 5 μl of the above solution was exposed to pulsed laser with different treatment time. After laser treatment, bacterial solution was collected into 985 μl of sterile pre-warmed TSB, supplemented with 10 μl of FITC-dextran (1 mg/ml), and incubated for 30 min at 37° C. The integrated fluorescence signal from an aliquot of the bacterial solution with or without laser treatment was measured through a plate reader with excitation of 488 nm and emission of 520 nm, respectively. Meanwhile, after incubation, the bacterial solution was spun down, got rid of the supernatant, washed with pre-warmed TSB twice, and fixed with 10% formalin. Structured illumination microscopy was conducted to quantify FITC-dextran uptake and its distribution on cell membrane with an excitation wavelength of 488 nm. Quantitative analysis of fluorescence emission intensity from individual MRSA cells was performed among groups with different laser treatment time.

### 10. Gentamicin-Texas Red Intracellular Uptake Assay

To study laser-mediated intracellular uptake of gentamicin (a representative of aminoglycoside), gentamicin was conjugated with a fluorescent dye, Texas-red, to form gentamicin-Texas red. Briefly, 10 mg of gentamicin was dissolved into 1 ml of 0.1 M sodium bicarbonate buffer (58761-500ML, Sigma Aldrich). 10 mg/ml of Texas red-X succinimidyl ester (T6134, Thermo Fisher Scientific) was added to the gentamicin solution slowly drop by drop. Then the mixed solution was stirred at room temperature for 1 hour. Gentamicin-Texas red was purified through sufficient dialysis against 0.1 M sodium bicarbonate buffer in a dialysis sack (Slide-A-Lyzer G2 Dialysis Cassettes, 2K MWCo, 15 mL, 87719, Thermao Fisher Scientific), and harvested through lyophilization (Labconco). Next, 1 ml of stationary-phase MRSA (˜10⁸ cells/ml) was spun down, got rid of the supernatant, and suspended with 100 μl of sterile 1×PBS. 5 μl of the above solution was exposed to pulsed laser for different treatment time (1 cm diameter illumination area, 120 mW). After treatment, bacterial droplet was collected into 985 μl of sterile 1×PBS, and then add 10 μl of 1 mg/ml Gentamicin-Texas red. Mixed solution was incubated at 37° C. for 30 min with a shaking speed of 200 rpm. After incubation, MRSA pellets were harvested through washing with sterile 1×PBS twice and then fixed with 10% formalin. Visualization of gentamicin-Texas red on bacterial cells was achieved through a confocal laser scanning microscope (FV 3000, Olympus) with the excitation wavelength of 514 nm. Quantitative analysis of fluorescence emission intensity from individual MRSA cells was conducted and allocated among groups with different laser treatment time.

### 11. Ciprofloxacin Intracellular Uptake Assay

To understand how laser treatment affects the uptake of ciprofloxacin (a representative of fluoroquinolone), we adopted a protocol published elsewhere³⁸. Briefly, 1 ml of stationary-phase MRSA (˜10⁸ cells/ml) was spun down, got rid of the supernatant, and suspended with 100 μl of sterile 1×PBS. 5 μl of the above solution was exposed to pulsed laser for different treatment time (1 cm diameter illumination area, 120 mW). After treatment, bacterial droplet was collected into 994 μl of sterile 1×PBS, and then added 1 μl of 10 mg/ml of ciprofloxacin (17850-5G-F, Sigma Aldrich), then incubated for 30 min at 37° C. with a shaking speed of 200 rpm. After incubation, MRSA pellets were washed twice by 2 ml of ice-cold PBS. Then ciprofloxacin was extracted using 1 ml of glycine (G8898, Sigma Aldrich)-HCl buffer at pH=3 for 2 hours. The amount of ciprofloxacin was estimated and quantified by measuring the fluorescence intensity via a plate reader with an excited wavelength of 275 nm and emission wavelength of 410 nm.

### 12. DiIC₁₈ Membrane Fluidity Assay

DiIC₁₈ is a fluorescent dye that displays affinity for membrane areas with increased fluidity due to its short hydrocarbon tail²⁴. In our protocol, briefly, 1 ml of stationary-phase MRSA (˜10⁸ cells/ml) were spun down, got rid of the supernatant, and suspended with 100 μl of pre-warmed TSB supplemented with 1% DMSO. 5 μl of the above solution was exposed to pulsed laser for different treatment time. After treatment, bacterial droplets (with 2.5, 5, 10 min treatment time) were collected into 985 μl of pre-warmed TSB supplemented with 1% DMSO. 10 μl of DiIC₁₈ (stock: 10 mg/ml in DMSO) were added to the above solution, and incubated for 30 min at 37° C. After incubation, harvested MRSA pellets were washed with pre-warmed TSB supplemented with 1% DMSO for four times, then sandwiched the concentrated bacterial samples between a poly-prep cover slides (P0425, Sigma Aldrich) and a thin cover glass (48404-457, VWR international). A confocal laser scanning microscope (FV3000, Olympus) was applied to visualize and quantify DiIC₁₈ uptake at an excitation wavelength of 561 nm and via a 100×oil immersion objective (NA=1.35, Olympus).

### 13. Daptomycin-BODIPY Membrane Insertion Assay

To study how the membrane fluidity change affects the insertion of membrane-targeting antibiotics, we applied daptomycin-BODIPY membrane insertion assay detailed as below. Firstly, we conjugated daptomycin with a fluorescent dye, BODIPY STP ester (B10006, Thermo Fisher Scientific). Briefly, 10 mg of daptomycin (103060-53-3, Acros Organics) was dissolved into 1 ml of 0.1 M sodium bicarbonate solution. Then 100 μl of BODIPY STP ester (B10006, Thermo Fisher Scientific, stock: 1 mg/ml in DMSO) was added to the daptomycin solution drop by drop. Then the mixed solution reacted under stirring at room temperature for 1 hour. Afterwards, the solution was under overnight dialysis against extensive 0.1 M sodium bicarbonate solution. After dialysis, the mixed solution was lyophilized. To further label MRSA cell membrane with daptomycin-BODIPY, 1 ml of stationary-phase MRSA (˜10⁸ cells/nil) was spun down, got rid of the supernatant, and suspended with 100 μl of sterile 1×PBS. 5 μl of the above solution was exposed to pulsed laser with different treatment time. After treatment, bacterial droplets were collected into 985 μl of sterile pre-warned TSB medium containing 150 μg/ml of CaCl₂. 10 μl of daptomycin-BODIPY (stock: 3 mg/ml in 1×PBS) was added to the above solution, and incubated for 30 min at 37° C. After incubation, harvested MRSA pellets were washed with 1×PBS twice, and fixed with 10% formalin. Confocal laser scanning microscope (FV3000, Olympus) was conducted to quantify daptomycin-BODIPY distribution and its signal intensity at an excitation wavelength of 488 nm. Quantitative analysis of the signal from individual MRSA cells was performed among groups with different laser treatment time.

### 14. PBP2a Immunofluorescence Assay

Basically, 1 ml of stationary-phase MRSA (˜10⁸ cells/ml) was spun down, got rid of the supernatant, and suspended with 100 μl of sterile 1×PBS. 5 μl of the above solution was exposed to pulsed laser for different treatment time. After treatment, bacterial droplets were collected into 980 μl of sterile 1×PBS, and 20 μl of a primary antibody (Rabbit Anti-PBP2a, RayBiotech, 130-10073-20, 10 μg/ml) targeting PBP2a was added to the above solution. Then the mixed solution was incubated for 30 min at 37° C. with a shaking speed of 200 rpm. After incubation, MRSA pellets were washed twice with sterile 1×PBS. As the last wash, MRSA pellets were suspended with 990 μl of 1×PBS. Then 10 μl of secondary antibody (Goat anti-Rabbit Cy5, Abcam, ab97077, 0.5 mg/ml) was added to the above solution, incubated for another 30 min at 37° C. with a shaking speed of 200 rpm. After incubation, MRSA pellets were washed with sterile 1×PBS twice and fixed with 10% formalin. Immunofluorescence experiment was conducted by a confocal laser scanning microscope at an excitation wavelength of 650 nm. Quantitative analysis of signal intensity and its distribution from individual MRSA cells was performed among groups with different laser treatment time.

### 15. PBP2a Western Blotting Assay

Briefly, 3 ml of stationary-phase MRSA (˜10⁸ cells/ml) was spun down and suspended with 100 μl of 1×PBS. 20 μl of the mixed solution was aliquoted to a centrifuge tube (89166-280, VWR international), and then exposed to pulsed laser with different treatment time (control, 5 min, 10 min, 20 min). After exposure, the four tubes containing MRSA solution were spun down at a speed of 13,000×g for 10 min at 4° C. Then the supernatants were collected into four new sterile tubes. To extract proteins from MRSA pellets, after removing the supernatant, MRSA pellets were suspended with 100 μl of lysis buffer (96.8 μl of RIPA, 1 μl 500 mM DTT, 1 μl of 10% Triton-X, 1 μl of protease inhibitor, and 1 μl of phosphorylase inhibitor). Then the mixed solutions were sonicated by a sonication probe (Cole-Parmer) at 4° C. Released proteins were harvested from the supernatants by centrifuging at 13,000×g for 10 min at 4° C. Electrophoresis separation of the proteins from both MRSA pellets and supernatants was conducted on a 12% SDS-PAGE gel (stacking gel: 4%) at a voltage of 50 V for 30 min followed by 100 V for 1 hour in 1×running buffer (1610772, Bio-Rad). After separation of the proteins, gels were transferred to a PVDF membrane (1620184, Bio-Rad) at a current of 150 mA overnight at 4° C. in 1×transfer buffer (1610771, Bio-Rad). After transferring, PVDF membrane was harvested and put into a clean plastic reservoir containing 5% milk solution (1706404, Bio-Rad). Then the plastic reservoir was placed on a rocking shaker for 30 min. After blocking, the PVDF membrane was further labelled with primary antibody (Rabbit anti-PBP2a, 1:500 dilution in 5% milk solution) for 2 hours in a rotary shaker. Then the PVDF membrane was washed with 1×washing buffer three times with each time for 5 min on the rotary shaker. Afterwards, the PVDF membrane was conjugated with a fluorescent secondary antibody (Eu-anti-Rabbit, Molecular Devices, 1:1000 dilution in 5% milk solution) for 1 hour on the rotary shaker and then washed with 1×washing buffer three times with each time for 5 min on the rotary shaker. Lastly, the protein-antibody-antibody fluorophore complex was detected through a plate reader at an excitation wavelength of 340 nm.

### 16. Membrane Computational Method

The Coarse-Grained (CG) simulations were performed using the MARTINI forcefield. The parameters for the cardiolipin were taken from the MARTINI database³⁹. For PBP2a, only the transmembrane helix was included in this simulation as we focus on the membrane properties in the current work. The saturated and unsaturated tail of the STX lipid were modeled by “C1” and “C4” bead type, respectively following other lipid parameters within the MARTINI model. The head group of the STX lipid is a glucose for which the MARTINI parameters were taken from the database. The bond and angle parameters for the CG beads of the STX tails were determined using structural information obtained from atomistic simulations. A single STX lipid in solution was simulated using the all atom CHARMM27⁴⁰ forcefield and the TIP3P⁴¹ water model. The equilibrium bond length and angle for the STX tail CG beads were obtained from the positions of the mass centers of the corresponding groups in atomistic simulations. The bond force constants for both the saturated and unsaturated tails and the angle force constants for the saturated tail were taken as same as for the other lipids in the MARTINI model. However, since every other bond in the unsaturated tail is a C═C bond, the tail is expected to be very rigid. So, the angle force constants for the unsaturated tail were taken to be higher (200 kJ/mol−rad²) than the angle force constants for the saturated tail (25 kJ/mol−rad²). To model STX following its photolysis, the long unsaturated tail was truncated, as suggested by the complete loss of C═C vibrational peak in the Raman spectra after STX photolysis. The transmembrane helix of the PBP2a protein was generated using the Chimera software⁴². The CG parameters for the peptide were generated using a script provided in the MARTINI database. We built a bilayer (˜17×17 nm²) of randomly mixed STX, cardiolipin and peptides (400:200:36). The built system was then solvated using the MARTINI water model; 10% anti-freezing beads were also added to avoid any artificial water freezing. Sodium and chlorine ions were then added to maintain 150 mM salt concentration. Each system (with full and truncated STX lipids, respectively) was equilibrated and simulated under the constant pressure and constant temperature ensemble for 10 μs. All simulations were conducted using the GROMACS program⁴³.

The RDF or the pair correlation function, g(r), between molecule type A and molecule type B is calculated using the following equation

$$g ( r ) = \frac{1}{< {ρ}_{B} >} \frac{1}{{N}_{A}} {\sum }_{i}^{{N}_{A}} {\sum }_{j}^{{N}_{B}} \frac{δ ( {r}_{ij} - r )}{4 π {r}^{2}}$$

Here, N_{A} and N_{B} are the number of molecules of type A and type B, respectively. ρ_{B} denotes the density of molecule type B in a sphere of radius rₘ around the molecule type A and <ρ_{B}> is the average of ρ_{B} calculated over all type A molecules. The rₘ was taken to be ˜6 nm which is half of the shortest box dimension.

The area expansion modulus K_{A} of the membrane was calculated using the following equation:

$${K}_{A} = \frac{{k}_{B} T < A >}{< δ {A}^{2} >}$$

Here k_{B}, T, and A are the Boltzmann constant, absolute temperature and the membrane surface area, respectively; <(δA²> represents the fluctuation in the surface area, which was calculated as <δA²>=<(A−<A>)²>, where <A> is the mean value of the surface area averaged over ˜5 μs simulation. The thermal fluctuations in the membrane surface area is less in a tightly packed membrane. Thus, a higher value of K_{A} represents a more tightly packed membrane.

### 17. Bacterial Growth Kinetics

To monitor the response of bacteria to laser treatment alone, antibiotic treatment alone, or their combinations, bacterial growth was continuously monitored overnight (18 hours with an interval of 30 min at 37° C.) by measuring optical density at 600 nm (OD₆₀₀). Depending on the specific assay applied, the bacterial cells were suspended in 100 or 200 μl TSB medium under different treatment schemes (antibiotic alone, laser treatment alone, antibiotic plus laser treatment) with a final concentration of ˜10⁵ CFU/ml. Bacterial growth was defined as OD₆₀₀≥0.1.

### 18. Minimal Inhibitory Concentration Measurement

The MICs of antibiotics were determined by the standard broth-dilution method recommended by the Clinical and Laboratory Standards Institute⁴⁴. Briefly, bacterial strains were grown aerobically overnight on tryptic soy agar (TSA, 22091, Sigma Aldrich) plates at 37° C. Bacterial colonies were then suspended into TSB medium with a concentration of ˜10⁵ CFU/ml and then transferred into 96-well plates (71000-078, VWR international). Antibiotics were added in the first row of the 96-well plates and then two-fold serially diluted. Plates were then incubated aerobically at 37° C. for ˜18 hours. MICs reported were the minimum concentration of antibiotics that completely inhibited the visual growth of the bacteria or with OD₆₀₀ less than 0.1 monitored by a plate reader (SpectraMax i3×, Molecular Devices). For each measurement, three independent replicates were applied. Table 1 shows the MICs of selected antibiotics against the tested bacterial strains.

### 19. Colony-Forming-Unit Enumeration Assay

To quantify viable bacterial cells, CFU experiments were performed. 100 μl of sample analyte was transferred into a 96-well plate and then three or four ten-fold serial dilution achieved by transferring 20 μl bacterial culture into 180 μl 1×PBS in the next dilution row. After serial dilution, an aliquot (4 μl) from each well was spotted onto sterile TSA plates. After incubating the plates overnight (˜18 hours) at 37° C., the colonies were enumerated, and cell number was calculated in CFU/ml. For each CFU enumeration experiment, three independent replicates were applied.

### 20. Post-Exposure and Post-Antibiotic Assays

To study the post-exposure effect for laser treatment, stationary-phase MRSA was prepared, washed and resuspended in 1×PBS at its original concentration. An aliquot (5 μl) of the bacterial suspension was transferred onto a glass cover slide (48393-230, VWR international) and treated by pulsed laser for different treatment time (1 cm-diameter illumination area, 100 mW). After treatment, the droplets were collected and resuspended 1:1000 into 5 ml of TSB medium for each group. An aliquot of 100 μl was then transferred to a 96-well plate for growth monitoring.

To study the post-antibiotic effect of antibiotics, we adopted a protocol published elsewhere⁴⁵. Briefly, stationary-phase MRSA (˜10⁸ cells/ml) were prepared, washed and cultured in fresh TSB at its original concentration supplemented with 4×MIC of antibiotics including ofloxacin, oxacillin and gentamicin for one hour at 37° C. A tube containing the untreated bacterial cells served as a control. Afterwards, antibiotics were washed out and 1:1000 diluted in TSB. An aliquot of 100 μl was then transferred to a 96-well plate for growth monitoring. Three independent replicates were applied for each antibiotic and/or laser-treated groups. Post-antibiotic effect was estimated by the difference between the times that required for both the control and antibiotic-treated groups to reach OD₆₀₀=0.3.

### 21. Checkerboard Broth Dilution Assay

Stationary-phase bacterial cells was washed and resuspended in 1×PBS at its original concentration. An aliquot (5 μl) of the bacterial solution (used as a control group) was transferred onto a glass cover slide as a droplet of ˜5 mm in diameter and exposed to pulsed laser for different treatment time (1-cm diameter illumination area, 100 mW). The treated droplet was collected and resuspended into 5 ml TSB (1:1000 dilution) for each group. Corresponding groups without laser treatment were also conducted for comparison. The bacterial suspensions were transferred to a 96-well plate with antibiotics supplemented into with the first row of the 96-well plate for eight two-fold serial dilution starting at a desired antibiotic concentration (e.g. ofloxacin: 2 μg/ml). After serial dilution, bacterial growth within the same well plate was monitored by a plate reader for 18 hours (OD₆₀₀, 37° C.). The checkerboard assay was used for groups with laser treatment alone or laser plus antibiotic treatment. Two independent experiments of checkerboard assay were performed for each antibiotic with or without laser treatment. Based on the readout of OD₆₀₀, a heat map was created to evaluate the antibiotic potentiation or synergistic effect enabled by STX photolysis.

### 22. Synergy Evaluation Between Antibiotic and Laser Treatment

Based on the checkerboard results, the fractional inhibitory concentration index (FICI), a synergy evaluation method between two antibiotics, was calculated as below: FICI=MIC of antibiotic A in combination/MIC of antibiotic A alone+MIC of antibiotic B in combination/MIC of antibiotic B alone. The interaction of the two antibiotics was defined as below: synergy if FICI≤0.5, no interaction if 0.5<FICI≤4, antagonism if FICI>4⁴⁶. As this demonstrated phototherapy approach depletes STX virulence instead of completely inhibiting bacterial growth, thus there is no MIC for laser treatment alone. Considering this reason, the synergy calculation was simplified as below: FICI=MIC of antibiotic A in combination with laser treatment/MIC of antibiotic A alone with synergy defined by FICI≤0.5.

### 23. Time-Killing Assay

Stationary-phase MRSA was prepared, washed and resuspended in 1×PBS at two-times of its original concentration. An aliquot (5 μl) of the MRSA suspension was transferred onto a glass cover slide as a droplet of ˜5 mm in diameter and exposed to pulsed laser for different treatment time (1 cm diameter illumination area, 100 mW). After laser treatment, the droplets were resuspended into 200 μl of 1×PBS (1:40 dilution) supplemented with antibiotics at different concentrations in a mini centrifuge tube (89166-278, VWR international). For example, daptomycin was added into MRSA solution after laser treatment at desired concentration of 0×MIC, 5×MIC, 10×MIC, 30×MIC, or 100×MIC supplemented with 50 μg/ml CaCl₂). Corresponding groups without laser treatment were also conducted for comparison. These tubes were incubated within an orbital incubator (37° C., 200 rpm) for different incubation time. At each specific time point, 40 μl of aliquot from each group was transferred to a 96-well plate for follow-up CFU enumerating assay. In the case of tobramycin, additional antibiotic washing by 1×PBS was performed before the CFU experiment to avoid antibiotic interference. For time-killing assay in fresh human whole blood, similar protocol was followed as above, except that 1×PBS was replaced by fresh human whole blood and the initial stationary-phase MRSA solution was diluted by ten times with a concentration of ˜10⁷ CFU/ml. The time-killing assay for hydrogen peroxide also followed the same protocol except replacing supplemented antibiotic by low-concentration hydrogen peroxide.

### 24. Serial Passage Assay for Resistance Development

To understand whether laser treatment could cause genotypic or phenotypic change in MRSA, and whether STX photolysis could reduce the resistance development for conventional antibiotics, a serial passage study for each treatment scenario was conducted. The initial generation (Day 1) used in this study was stationary-phase MRSA. The sample was prepared, washed and resuspended in 1×PBS at its original concentration. An aliquot (5 μl) of the MRSA suspension was transferred onto a glass cover slide as a droplet of ˜5 mm in diameter with or without 10 min laser treatment (1 cm diameter illumination area, 120 mW). The droplets were then collected and resuspended into 5 ml of TSB medium (1:1000 dilution) with an estimated cell concentration of 10⁵ CFU/ml. To study resistance development or selection induced by laser treatment alone, three groups were included: a group without laser treatment (SPO), a group with laser treatment (SPL1), and another independent group with laser treatment as a duplicate (SPL2). To study resistance development induced by antibiotic treatment alone and laser plus antibiotic treatment, three groups were included for each antibiotic: antibiotic alone-treated group (SPA0), laser plus antibiotic-treated group (SPLA1), and another laser plus antibiotic-treated group as another independent serial passage (SPLA2). For SPO, SPL1, and SPL2, 200 μl of bacterial suspension was directly transferred to each well of a 96-well plate, with three replicates conducted for each group. For SPA0, SPLA1, and SPLA2, 200 μl of bacterial suspension was transferred into the first dilution row of a 96-well plate with supplemented antibiotics at a desired starting concentration, whereas 100 μl of bacterial suspension was transferred to the rest dilution rows. After twelve two-fold serial dilution, 100 μl of bacterial suspension was added into each well to make a 200 μl of final volume for each well, thus, as an example, supplementing 5.12 μl of 10 mg/ml ofloxacin solution into 200 μl of bacterial culture in the first dilution row makes a starting concentration of 128 μg/ml. Three replicates were applied for each group. These well plates were incubated in a shaker at 37° C. and 200 rpm for 18 hours followed by OD₆₀₀ measurement by a plate reader. After MICs recording for each group, the well plates were continuously incubated in the shaker for 3 days in total. On Day 4, 200 μl of bacterial sample from each group was collected, washed, and resuspended in 1×PBS at its original concentration used as new inoculum for the next passage following the same protocol as described above. Samples for SPA0, SPLA1, and SPLA2 groups were collected from wells supplemented with sub-MIC antibiotic. Samples for SPO, SPL1, and SPL2 groups were also collected from the well plates. The left bacterial suspension for each group was stored in 25% glycerol at −80° C. for subsequent analysis and experiments. Serial passage for all groups were performed for 50 days with 16 generations in total. Raman spectroscopy was then applied to monitor STX expression level in groups of interest after the entire serial passage experiment. The protocol is detailed as below: 100 μl of ˜400 μl stored bacterial culture was collected, spun down with the supernatant being removed, then resuspended into 5 μl 1×PBS as high-concentration bacterial solution (20 times concentrated). An aliquot (1 μl) was transferred and then sandwiched between two glass cover slides for STX quantification by resonance Raman spectroscopy.

### 25. In Vivo Mice Infection Model

The in vivo mice experiment was conducted following protocols approved by Boston University Animal Care and Use Committee (BUACUC). To initiate the formation of a skin wound, five groups (N=5) of eight-week-old female BALB/c mice (obtained from the Jackson Laboratory, ME, USA) were disinfected with ethanol (70%) and shaved on the middle of their back (approximately a one-inch by one-inch square region around the injection site) one day prior to infection as described from a reported procedure⁴⁵. To prepare the bacterial inoculum, an aliquot of overnight culture of MRSA USA300 was transferred to fresh TSB and shaken at 37° C. until an OD₆₀₀ value of ˜1.0 was achieved. The cells were centrifuged, washed once with 1×PBS, re-centrifuged, and then re-suspended in 1×PBS. Mice subsequently received an intradermal injection (40 μl) containing ˜10⁹ CFU/ml MRSA USA300. An open wound formed at the site of injection for each mouse, ˜48 hours post-infection. Topical treatment was initiated subsequently with each group of mice receiving the following: daptomycin (1%, using glycerol as the vehicle), pulsed laser (1 cm diameter illumination area, 10 min treatment time, 120 mW), or a combination of pulsed laser and daptomycin. One group of mice was left as the control. Each group of mice receiving a particular treatment regimen was housed separately in a ventilated cage with appropriate bedding, food, and water. Mice were checked twice daily during infection and treatment to ensure no adverse reactions were observed. Mice were treated once daily (once every 24 hours) for three days, before they were humanely euthanized via CO₂ asphyxiation 12 hours after the last dose was administered. The region around the skin wound was lightly swabbed with ethanol (70%) and excised. The tissue was subsequently homogenized in 1×PBS. The homogenized tissue was then serially diluted in 1×PBS before plating onto mannitol salt agar plates (S. aureus specific). Plates were incubated for at least 19 hours at 37° C. before CFU assay for each group. Outlier was removed based upon the Dixon Q Test. Data were analyzed via an unpaired t-test, utilizing Origin 2019b (OriginLab Corporation).

### 26. H&E Histology Analysis of Mice Skin

To evaluated phototoxicity of laser treatment on healthy mice skin, mice (N=3) were treated with pulsed laser once daily for three days. After treatments, mice were humanely euthanized under CO₂ asphyxiation. Treated mice skin were sacrificed and collected into 10% formalin solution. H&E (hematoxylin and eosin) staining were utilized to stain sacrificed mice skin. Skin slices were imaged and analyzed by Boston University Experimental Pathology Service Core.

### 27. Phototoxicity on Human Cell Line

To evaluate the toxicity of pulsed laser, we chose a human cell line (human epithelial keratinocyte cells, HEK 293) to evaluate the phototoxicity. HEK cells were cultured at Dulbecco's Modified Eagle Medium (DMEM, Thermo Fisher Scientific) supplemented with 10% fetal bovine serum. A colorimetric MTT assay was used to assess the cell metabolic activity. Briefly, 5 mg of MTT (M6494, Thermo Fisher Scientific) was dissolved in 1 ml 1×PBS. Then MTT solution was diluted with serum-free DMEM medium at ratio of 1:10. The pulsed laser was applied to treat HEK 293 cells in a 96-well plate. After treatment, 100 μl of the diluted MTT solution (pre-warmed) was added to each treated well, and then incubated for four hours in dark at 4° C. After incubation, supernatants were removed, and 200 μl of DMSO was added to the wells. OD₅₄₀ from each treated well was measured by a plate reader.

### 28. Statistical Analysis

Statistical analysis was conducted through unpaired t-test. **** means significantly different with the p-value<0.0001. *** means significantly different with the p-value<0.001. ** means significantly different with the p-value<0.01. * means significantly different with the p-value<0.05. ‘ns’ means no significant difference.

1. A treatment regimen for sensitizing antibiotic-resistance Staphylococcus aureus, comprising a means to targeted photo-bleach the yellow pigment of staphyloxanthin (STX) with short-pulsed blue laser or low-level blue light, an effective amount of oxidative agent or an effective amount of antibiotics.

2. The treatment regimen according to claim 1, wherein the short-pulsed blue laser is nanosecond pulsed laser.

3. The treatment regimen according to claim 1, wherein the oxidative agent is hydrogen peroxide.

4. The treatment regimen according to claim 1, wherein the antibiotic-resistant Staphylococcus aureus is selected from the group consisting of methicillin-resistant Staphylococcus aureus (MRSA), vancomycin-resistance S. aureus (VRSA), sulfamethoxazole/trimethoprim-resistant MRSA (Sul/Tri-R MRSA), and erythromycin-resistant MRSA (Ery-R MRSA).

5. The treatment regimen according to claim 1, wherein the effective amount of antibiotics are selected from the group consisting of penicillins, quinolones, tetracyclines, aminoglycosides, lipopeptides, and oxazolidinones.

6. The treatment regimen according to claim 5 prevents the development of S. aureus resistance to ciprofloxacin and ofloxacin.

7. The treatment regimen according to claim 5 delays the development of S. aureus resistance to linezolid, tetracycline and tobramycin.

8. A portable device to sequentially or simultaneously provide short-pulsed blue laser or low-level blue light to the lesion of a patient with antibiotic-resistant S. aureus infection and to administer an effective amount of hydrogen peroxide or antibiotics.

9. The portable device according to claim 8, wherein the patient is provided an effective amount of antibiotics selected from the group consisting of penicillins, quinolones, tetracyclines, aminoglycosides, lipopeptides, and oxazolidinones.

10. A method to treat a patient infected by antibiotic-resistant S. aureus, comprising: providing to the patient with short-pulsed blue laser or low-level blue light at the lesion to photo-bleach the yellow pigment of staphyloxanthin (STX), administering effective amount of oxidative agents at the lesion, or administering an effective amount of antibiotics.

11. The method according to claim 10, wherein the short-pulsed blue laser is nanosecond pulsed laser.

12. The method according to claim 10, wherein the oxidative agent is hydrogen peroxide.

13. The method according to claim 10, wherein the antibiotic-resistant Staphylococcus aureus is selected from the group consisting of methicillin-resistant Staphylococcus aureus (MRSA), vancomycin-resistance S. aureus (VRSA), sulfamethoxazole/trimethoprim-resistant MRSA (Sul/Tri-R MRSA), and erythromycin-resistant MRSA (Ery-R MRSA).

14. The method according to claim 10, wherein the effective amount of antibiotics are selected from the group consisting of penicillins, quinolones, tetracyclines, aminoglycosides, lipopeptides, and oxazolidinones.

15. The method according to claim 10, wherein the patient lesion is a wound.

16. The method according to claim 10, wherein the patient has an ear infection.
